SEQUENCE LISTING (30 sequences)

SEQ ID NO: 1 - protein, 39 aa, synthetic construct
  SITE 1: note = H
  SITE 3: note = Q
  VARIANT 10: note = K
  VARIANT 10: note = Y
  VARIANT 12: note = E
  VARIANT 12: note = K
  VARIANT 12: note = I
  VARIANT 13: note = Y
  VARIANT 16: note = S
  VARIANT 16: note = E
  VARIANT 16: note = A
  VARIANT 17: note = E
  VARIANT 17: note = R
  VARIANT 17: note = K
  VARIANT 18: note = R
  VARIANT 18: note = K
  VARIANT 18: note = A
  VARIANT 19: note = A
  VARIANT 20: note = R
  VARIANT 20: note = Q
  VARIANT 20: note = K
  VARIANT 21: note = D
  VARIANT 21: note = E
  VARIANT 23: note = V
  VARIANT 23: note = I
  VARIANT 24: note = A
  VARIANT 24: note = Q
  VARIANT 24: note = E
  SITE 25: note = W
  VARIANT 27: note = E
  VARIANT 27: note = L
  VARIANT 28: note = A
  VARIANT 28: note = D
  VARIANT 28: note = E
  VARIANT 29: note = G
  VARIANT 29: note = T
  VARIANT 2: note = serine methyl ether
  VARIANT 2: note = Aib
  VARIANT 2: note = D-Serine
  VARIANT 30: note = absent
  VARIANT 30: note = G
  VARIANT 31: note = absent
  VARIANT 31: note = P
  VARIANT 32: note = absent
  VARIANT 32: note = S
  VARIANT 33: note = absent
  VARIANT 33: note = S
  VARIANT 34: note = absent
  VARIANT 34: note = G
  VARIANT 35: note = absent
  VARIANT 36: note = absent
  VARIANT 37: note = absent
  VARIANT 38: note = absent
  VARIANT 39: note = absent
  VARIANT 10: note = K is acylated with Moiety I; note = K is acylated with Moiety A; note = K is acylated with Moiety E; note = K is acylated with Moiety D; note = K is acylated with Moiety B; note = K is acylated with Moiety H; note = K is acylated with Moiety C
  VARIANT 20: note = K is acylated with Moiety A, B, C, D, E, H, or I.
  VARIANT 13: note = Nva; note = Serine methyl ether; note = Nle; note = Y; note = 2-amino-2,4-dimethylpentanoic acid
XXXGTFTSDXSXXLDXXXXXXFXXXLXXXXXXXXXXXXX

SEQ ID NO: 2 - protein, 39 aa, synthetic construct; identical to SEQ ID NO: 1
  VARIANT 1: note = H
  VARIANT 2: note = D-serine methyl ether
  VARIANT 2: note = S
  VARIANT 2: note = Aib
  VARIANT 2: note = D-Serine
  VARIANT 3: note = Q
  VARIANT 10: note = K
  VARIANT 10: note = Y
  VARIANT 12: note = E
  VARIANT 12: note = K
  VARIANT 12: note = I
  VARIANT 16: note = S
  VARIANT 16: note = E
  VARIANT 16: note = A
  VARIANT 17: note = E
  VARIANT 17: note = R
  VARIANT 17: note = K
  VARIANT 18: note = R
  VARIANT 18: note = K
  VARIANT 18: note = A
  VARIANT 19: note = A
  VARIANT 20: note = R
  VARIANT 20: note = Q
  VARIANT 20: note = K
  VARIANT 21: note = D
  VARIANT 21: note = E
  VARIANT 23: note = V
  VARIANT 24: note = A
  VARIANT 24: note = Q
  VARIANT 24: note = E
  VARIANT 25: note = W
  VARIANT 27: note = E
  VARIANT 27: note = L
  VARIANT 28: note = A
  VARIANT 28: note = D
  VARIANT 28: note = E
  VARIANT 29: note = G
  VARIANT 29: note = T
  VARIANT 30: note = absent
  VARIANT 30: note = G
  VARIANT 31: note = absent
  VARIANT 31: note = P
  VARIANT 32: note = absent
  VARIANT 32: note = S
  VARIANT 33: note = absent
  VARIANT 33: note = S
  VARIANT 34: note = absent
  VARIANT 34: note = G
  VARIANT 35: note = absent
  VARIANT 36: note = absent
  VARIANT 37: note = absent
  VARIANT 38: note = absent
  VARIANT 39: note = absent
  VARIANT 10: note = K is acylated with Moiety C; note = K is acylated with Moiety D; note = K is acylated with Moiety A; note = K is acylated with Moiety B
  VARIANT 20: note = K is acylated with Moiety B; note = K is acylated with Moiety C; note = K is acylated with Moiety A; note = K is acylated with Moiety D
  VARIANT 13: note = 2-amino-2,4-dimethylpentanoic acid; note = Nva; note = Y; note = Serine methyl ether; note = Nle

SEQ ID NO: 3 - protein, 30 aa, synthetic construct
  VARIANT 2: note = S
  VARIANT 2: note = D-serine methyl ester
  VARIANT 2: note = Aib
  VARIANT 10: note = K
  VARIANT 10: note = K is acylated with Moiety E; note = K is acylated with Moiety A; note = K is acylated with Moiety B; note = K is acylated with Moiety D; note = K is acylated with Moiety C
HXQGTFTSDXSEYLDSERARDFVAWLEAGG

SEQ ID NO: 4 - protein, 34 aa, synthetic construct
  SITE 2: note = Aib
  VARIANT 12: note = K
  VARIANT 12: note = I
  VARIANT 13: note = Y
  VARIANT 13: note = Nva
  VARIANT 20: note = K
  VARIANT 20: note = acylated with Moiety B; note = acylated with Moiety A; note = acylated with Moiety C; note = acylated with Moiety D
HXQGTFTSDYSXXLDEKKAXEFVEWLLEGGPSSG

SEQ ID NO: 5 - protein, 29 aa, synthetic construct
  SITE 2: note = D-Serine
  VARIANT 10: note = K
  VARIANT 10: note = acylated with Moiety A
  VARIANT 10: note = acylated with Moiety B
  VARIANT 10: note = acylated with Moiety C
  VARIANT 10: note = acylated with Moiety D
HXQGTFTSDXSKYLDARAAQDFVQWLLDT

SEQ ID NO: 6 - protein, 30 aa, synthetic construct
HSQGTFTSDKSEYLDSERARDFVAWLEAGG

SEQ ID NO: 7 - protein, 29 aa, synthetic construct
  SITE 2: note = D-Serine
HXQGTFTSDKSKYLDARAAQDFVQWLLDT

SEQ ID NO: 8 - protein, 34 aa, synthetic construct
  SITE 2: note = Aib
HXQGTFTSDYSKYLDEKKAKEFVEWLLEGGPSSG

SEQ ID NO: 9 - protein, 30 aa, synthetic construct; identical to SEQ ID NO: 6
  VARIANT 10: note = K is acylated with Moiety I; note = K is acylated with Moiety F; note = K is acylated with Moiety D; note = K is acylated with Moiety E; note = K is acylated with Moiety G; note = K is acylated with Moiety A; note = K is acylated with Moiety B; note = K is acylated with Moiety H; note = K is acylated with Moiety C

SEQ ID NO: 10 - protein, 30 aa, synthetic construct
  SITE 2: note = D-Serine methyl ester
  VARIANT 10: note = K is acylated with Moiety B; note = K is acylated with Moiety G; note = K is acylated with Moiety F; note = K is acylated with Moiety H; note = K is acylated with Moiety A; note = K is acylated with Moiety E; note = K is acylated with Moiety I; note = K is acylated with Moiety C; note = K is acylated with Moiety D
HXQGTFTSDKSEYLDSERARDFVAWLEAGG

SEQ ID NO: 11 - protein, 31 aa, synthetic construct
  SITE 2: note = Aib
  VARIANT 11: note = K is acylated with Moiety D; note = K is acylated with Moiety I; note = K is acylated with Moiety B; note = K is acylated with Moiety F; note = K is acylated with MoietyH; note = K is acylated with Moiety E; note = K is acylated with Moiety G; note = K is acylated with Moiety A; note = K is acylated with Moiety C
HXSQGTFTSDKSEYLDSERARDFVAWLEAGG

SEQ ID NO: 12 - protein, 29 aa, synthetic construct
  SITE 2: note = D-Serine
  VARIANT 10: note = K is acylated with Moiety A; note = K is acylated with Moiety I; note = K is acylated with Moiety H; note = K is acylated with Moiety B; note = K is acylated with Moiety C; note = K is acylated with Moiety E; note = K is acylated with Moiety G; note = K is acylated with Moiety D; note = K is acylated with Moiety F
HXQGTFTSDKSKYLDARAAQDFVQWLLDT

SEQ ID NO: 13 - protein, 34 aa, synthetic construct; identical to SEQ ID NO: 8
  SITE 2: note = Aib
  VARIANT 20: note = K is acylated with Moiety G; note = K is acylated with Moiety C; note = K is acylated with Moiety E; note = K is acylated with Moiety H; note = K is acylated with Moiety B; note = K is acylated with Moiety D; note = K is acylated with Moiety I; note = K is acylated with Moiety A; note = K is acylated with Moiety F

SEQ ID NO: 14 - protein, 34 aa, synthetic construct
  SITE 2: note = Aib
  SITE 13: note = Nva
  VARIANT 20: note = K is acylated with Moiety G; note = K is acylated with Moiety I; note = K is acylated with Moiety H; note = K is acylated with Moiety B; note = K is acylated with Moiety C; note = K is acylated with Moiety F; note = K is acylated with Moiety A; note = K is acylated with Moiety E; note = K is acylated with Moiety D
HXQGTFTSDYSKXLDEKKAKEFVEWLLEGGPSSG

SEQ ID NO: 15 - protein, 34 aa, synthetic construct
  SITE 2: note = Aib
  SITE 13: note = Nva
  VARIANT 20: note = acylated with Moiety A
  VARIANT 20: note = acylated with Moiety B
  VARIANT 20: note = acylated with Moiety C
  VARIANT 20: note = acylated with Moiety D
  VARIANT 20: note = acylated with Moiety E
  VARIANT 20: note = acylated with Moiety F
  VARIANT 20: note = acylated with Moiety G
  VARIANT 20: note = acylated with Moiety H
  VARIANT 20: note = acylated with Moiety I
HXQGTFTSDYSIXLDEKKAKEFVEWLLEGGPSSG

SEQ ID NO: 16 - protein, 30 aa, synthetic construct
  SITE 10: note = Moiety C
HSQGTFTSDXSEYLDSERARDFVAWLEAGG

SEQ ID NO: 17 - protein, 30 aa, synthetic construct; identical to SEQ ID NO: 3
  SITE 2: note = D-Serine methyl ester
  SITE 10: note = Moiety E

SEQ ID NO: 18 - protein, 30 aa, synthetic construct; identical to SEQ ID NO: 3
  SITE 2: note = Aib
  SITE 10: note = Moiety E

SEQ ID NO: 19 - protein, 30 aa, synthetic construct; identical to SEQ ID NO: 3
  SITE 2: note = Aib
  SITE 10: note = Moiety A

SEQ ID NO: 20 - protein, 34 aa, synthetic construct
  SITE 10: note = Moiety A
HXQGTFTSDYSKYLDEKKAXEFVEWLLEGGPSSG

SEQ ID NO: 21 - protein, 34 aa, synthetic construct; identical to SEQ ID NO: 20
  SITE 2: note = Aib
  SITE 20: note = Moiety B

SEQ ID NO: 22 - protein, 34 aa, synthetic construct; identical to SEQ ID NO: 20
  SITE 2: note = Aib
  SITE 20: note = Moiety A

SEQ ID NO: 23 - protein, 34 aa, synthetic construct; identical to SEQ ID NO: 20
  SITE 2: note = Aib
  SITE 20: note = Moiety D

SEQ ID NO: 24 - protein, 34 aa, synthetic construct; identical to SEQ ID NO: 20
  SITE 2: note = Aib
  SITE 20: note = Moiety C

SEQ ID NO: 25 - protein, 34 aa, synthetic construct
  SITE 2: note = Aib
  SITE 13: note = Nva
  SITE 20: note = Moiety A
HXQGTFTSDYSKXLDEKKAXEFVEWLLEGGPSSG

SEQ ID NO: 26 - protein, 34 aa, synthetic construct
  SITE 2: note = Aib
  SITE 13: note = Nva
  SITE 20: note = Moiety A
HXQGTFTSDYSIXLDEKKAXEFVEWLLEGGPSSG

SEQ ID NO: 27 - protein, 34 aa, synthetic construct; identical to SEQ ID NO: 20
  SITE 2: note = Aib
  SITE 20: note = Moiety F

SEQ ID NO: 28 - protein, 23 aa, synthetic construct
PMLUCARELLIRONALDGTFTSD

SEQ ID NO: 29 - protein, 8 aa, synthetic construct
  SITE <1: note = Aib or Ser(OMe)
QGTFTSDK

SEQ ID NO: 30 - protein, 7 aa, synthetic construct
  SITE <1: note = Aib
QGTFTSD

(19) United States
(12) Patent Application Publication
(10) Pub. No.: US 2026/0356904 A1
(43) Pub. Date: Nov. 12, 2026

(54) NOVEL GLP-1/GIP DUAL AND GLP-1/GCG DUAL RECEPTOR AGONISTS

(71) Applicant: SUN PHARMACEUTICAL INDUSTRIES LIMITED, MUMBAI (IN)

(72) Inventors: Rajamannar THENNATI, Vadodara (IN); Vinod Sampatrao BURADE, Vadodara (IN); Muthukumaran NATARAJAN, Vadodara (IN); Dhiren Rameshchandra JOSHI, Vadodara (IN); Manish Harendraprasad GANDHI, Vadodara (IN); Chandulal Thakarshibhai JIVANI, Vadodara (IN); Abhishek TIWARI, Kota (IN); Krunal Harishbhai SONI, Vadodara (IN); Alpeshbhai Balabhai MARU, Bhavnagar (IN); Pankaj Vinodrao DIXIT, Indore (IN); Bharatbhai Balabhai PATELIYA, Vadodara (IN); Ravishankara Madavati NAGARAJA, Vadodara (IN)

(21) Appl. No.: 19/491,096

(22) PCT Filed: Jun. 7, 2024
(86) PCT No.: PCT/IB2024/055620

§ 371 Date: Dec. 8, 2025

(30) Foreign Application Priority Data
Jun. 9, 2023 (IN) 202321039646

(51) Int. Cl. C07K 14/575; A61K 38/00; A61P 3/10

(52) U.S. Cl. CPC C07K 14/575; A61P 3/10; A61K 38/00

(57) ABSTRACT

The present disclosure provides GLP-1/GIP and GLP-1/GCG dual receptor agonists comprising incretin analog polypeptides and use thereof in the treatment or prevention of Type 2 diabetes mellitus (T2DM), hyperlipidemia/dyslipidemia, metabolic syndromes, metabolic dysfunction-associated steatotic liver disease (MASLD), metabolic dysfunction- associated steatohepatitis (MASH), neurodegenerative disorders, fibrosis, cardiovascular risks, and/or obesity.

### CROSS-REFERENCE TO RELATED APPLICATIONS

This application claims the benefit of priority of Indian Application No. 202321039646, filed Jun. 9, 2023, which is incorporated herein by reference in its entirety.

### REFERENCE TO AN ELECTRONIC SEQUENCE LISTING

This application contains a Sequence Listing which has been submitted electronically and is hereby incorporated by reference in its entirety. The Sequence Listing was created on Jun. 7, 2024, is named “24-0805-WO_Sequence-Listing.xml” and is 110,592 bytes in size.

### FIELD

The present disclosure relates to glucagon-like peptide-1 (GLP-1) mono receptor agonists, GLP-1/glucose-dependent insulinotropic polypeptide or gastrointestinal peptide (GIP) dual receptor agonists, and GLP-1/glucagon (GCG) dual receptor agonists. In particular, the present disclosure relates to GLP-1/GIP and GLP-1/GCG dual receptor agonists comprising incretin analog polypeptides. The polypeptides as described herein have structural features that provide balanced activity and an extended duration of action at each of these receptors. The polypeptides according to the present invention may be useful for treating Type 2 diabetes mellitus (T2DM), hyperlipidemia/dyslipidemia, metabolic syndromes, metabolic dysfunction-associated steatotic liver disease (MASLD), metabolic dysfunction-associated steatohepatitis (MASH), neurodegenerative disorders, fibrosis, and/or obesity, and reducing cardiovascular risks.

### BACKGROUND

The prevalence of diabetes has continued to rise over the past several decades. T2DM is the most prevalent form of diabetes, which is characterized by high blood glucose levels caused by insulin resistance. A person suffering from T2DM is more likely to develop comorbidities such as hyperlipidemia/dyslipidemia, metabolic syndromes, metabolic dysfunction-associated steatotic liver disease (MASLD), metabolic dysfunction-associated steatohepatitis (MASH), neurodegenerative disorders, fibrosis, cardiovascular risks, and/or obesity.

The current therapies for T2DM include diet and exercise as well as treatment with oral medications and injectable glucose-lowering drugs including incretin-based therapies, such as GLP-1 mono receptor agonists and/or GLP-1/GIP dual receptor agonists. For example, Cotadutide (SEQ ID NO: 6), MK-1462 (SEQ ID NO: 7) and Mazdutide (SEQ ID NO: 8) are peptides, which act as GLP-1/GCG dual receptor agonists.

WIPO publication numbers WO2019/193576, WO2006/097537 and WO1998/008871 disclose GLP-1 mono receptor agonist compounds. WIPO publication numbers WO2022/079639, WO2021/260530, WO2017/74714A1, WO2020/23386, WO2020/023388, WO2015/067715, WO2016/111971, WO2014/192284, WO2011/119657, and WO2013/164483 disclose GLP-1/GIP dual receptor agonist compounds. WIPO publication numbers WO2011/075393, WO2012/177444, WO2014/091316, and WO2017/153575 disclose GLP-1/GCG dual receptor agonist compounds.

Recent studies on GLP-1/GCG dual and/or GLP-1/GIP/GCG triple receptor agonists have also highlighted the importance of understanding the contribution of individual hormone action and divergent effects by varying the GLP-1: GCG activity and the ratios in GLP-1/GCG dual receptor agonists and GLP-1/GIP/GCG triple receptor agonists.

Glucagon Receptor (GCGR) agonism, besides being diabetogenic, is known to increase heart rate and contractility, which might lead to adverse cardiovascular outcomes. Further chronic excess of glucagon also leads to catabolism of amino acids and proteins, which lead to a loss of lean body mass. These side effects offset the weight loss benefits provided by GCG agonism.

A delicate balance is required between GLP-1 and GCG receptor agonism to get optimum results with minimum side effects.

Cotadutide, which is now discontinued, also shows high potency towards GCGR in comparison to GLP-1R.

While the broad metabolic benefits of GLP-1 mono or GLP-1/GIP dual receptor agonist compounds have been established in the treatment paradigm, a need still remains for treatments, especially for T2DM, that are capable of providing effective glucose control with weight loss benefits and a favorable adverse effect profile. There is also a need for therapeutic agents available for use with sufficiently extended duration of action to allow for dosing as infrequently as twice-weekly or once a week.

### SUMMARY

In one aspect, the present disclosure provides a polypeptide or a pharmaceutically acceptable salt thereof comprising the amino acid sequence:

(SEQ ID NO: 1)
X1-X2-X3-G-T-F-T-S-D-X10-S-X12-X13-L-D-X16-X17-
X18-X19-X20-X21-F-X23-X24-X25-L-X27-X28-X29-X30-
X31-X32-X33-X34-X35-X36-X37-X38-X39

wherein:
   X1 is H;
   X2 is D-Ser(OMe), Aib or D-S;
   X3 is Q;
   X10 is K or Y;
   X12 is E, K or I;
   X13 is Y, S(OMe), nor-V, nor-L, or αMe-L;
   X16 is S, E or A;
   X17 is E, R or K;
   X18 is, K or A;
   X19 is A;
   X20 is R, Q or K;
   X21 is D or E;
   X23 is V or I;
   X24 is A, Q or E;
   X25 is W;
   X27 is E or L;
   X28 is A, D or E;
   X29 is G or T;
   X30 is absent or G;
   X31 is absent or P;
   X32 is absent or S;
   X33 is absent or S;
   X34 is absent or G;
   X35 is absent;
   X36 is absent;
   X37 is absent;
   X38 is absent; and
   X39 is absent;
wherein the acid group of the C-terminal amino acid is a free carboxylic acid group or is amidated as C-terminal primary amide; and
with a proviso that at least one of X10 and X20 is K, and further provided at least one of said K comprises a side chain amino (ε amino) group acylated with a moiety of the formula selected from:

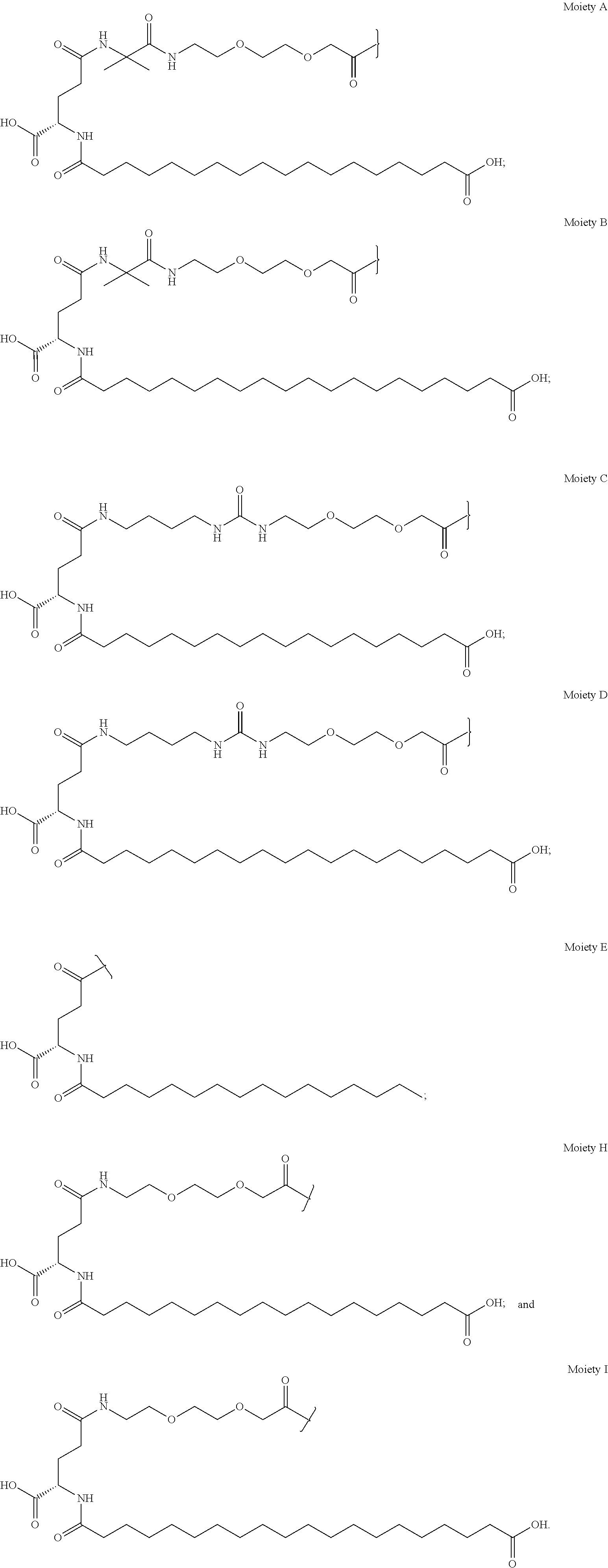

In another aspect, the present disclosure provides a polypeptide or a pharmaceutically acceptable salt thereof comprising the amino acid sequence:

(SEQ ID NO: 2)
X1-X2-X3-G-T-F-T-S-D-X10-S-X12-X13-L-D-X16-X17-
X18-X19-X20-X21-F-X23-X24-X25-L-X27-X28-X29-X30-
X31-X32-X33-X34-X35-X36-X37-X38-X39

wherein:
   X1 is H;
   X2 is S, D-Ser(OMe), Aib, or D-S;
   X3 is Q;
   X10 is K or Y;
   X12 is E, K or I;
   X13 is Y, S(OMe), nor-V, nor-L, or αMe-L;
   X16 is S, E or A;
   X17 is E, R or K;
   X18 is, K or A;
   X19 is A;
   X20 is R, Q or K,
   X21 is D or E;
   X23 is V;
   X24 is A, Q or E;
   X25 is W;
   X27 is E or L;
   X28 is A, D or E;
   X29 is G or T;
   X30 is absent or G;
   X31 is absent or P;
   X32 is absent or S;
   X33 is absent or S;
   X34 is absent or G;
   X35 is absent;
   X36 is absent;
   X37 is absent;
   X38 is absent; and
   X39 is absent;
wherein the acid group of the C-terminal amino acid is a free carboxylic acid group or is amidated as C-terminal primary amide; and
with a proviso that at least one of X10 and X20 is K, and further provided that at least one of said K comprises a side chain amino (ε amino) group acylated with a moiety of the

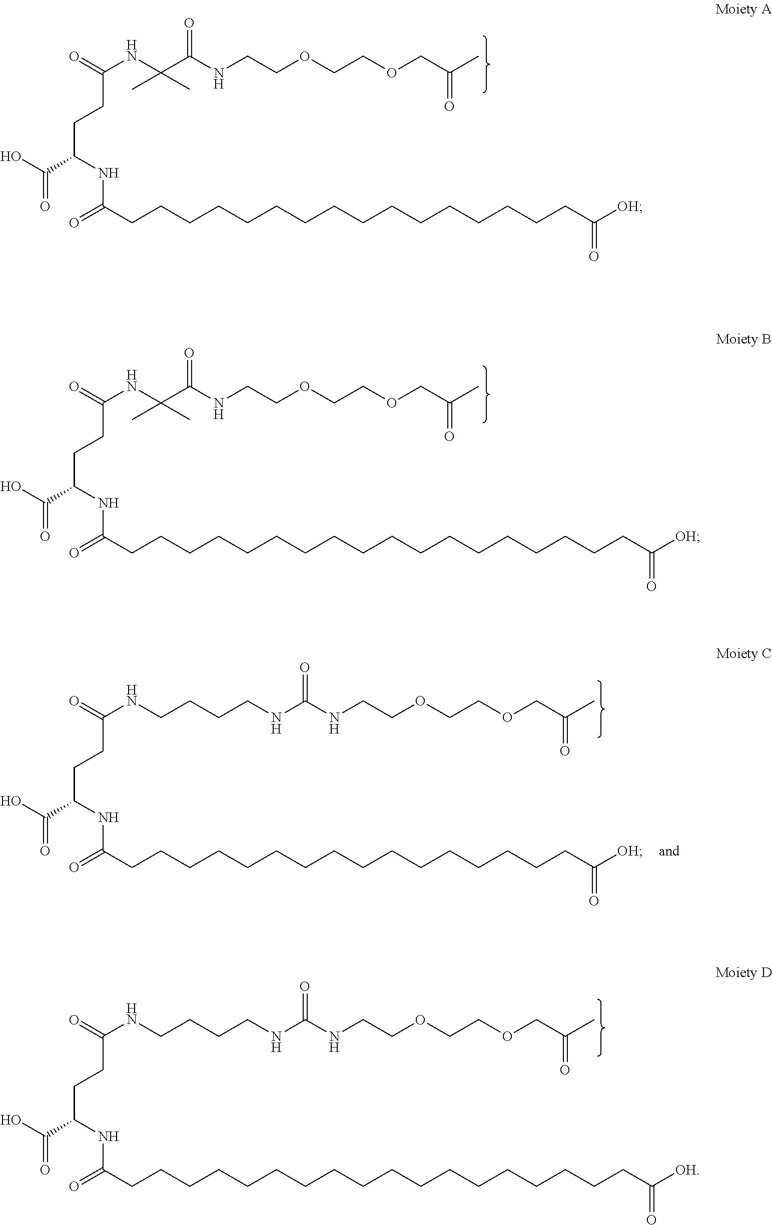 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

In another aspect, the present disclosure provides a polypeptide or a pharmaceutically acceptable salt thereof comprising the amino acid sequence:

(SEQ ID NO: 3)
H-X2-Q-G-T-F-T-S-D-X10-S-E-Y-L-D-S-E-R-A-R-D-F-V-
A-W-L-E-A-G-G

wherein:
   X2 is S, D-S(OMe) or Aib; and
   X10 is K;
wherein the acid group of the C-terminal amino acid is a free carboxylic acid group or is amidated as C-terminal primary amide;
wherein the side chain amino (ε amino) group of K at position X10 is acylated with a moiety of the formula selected from:

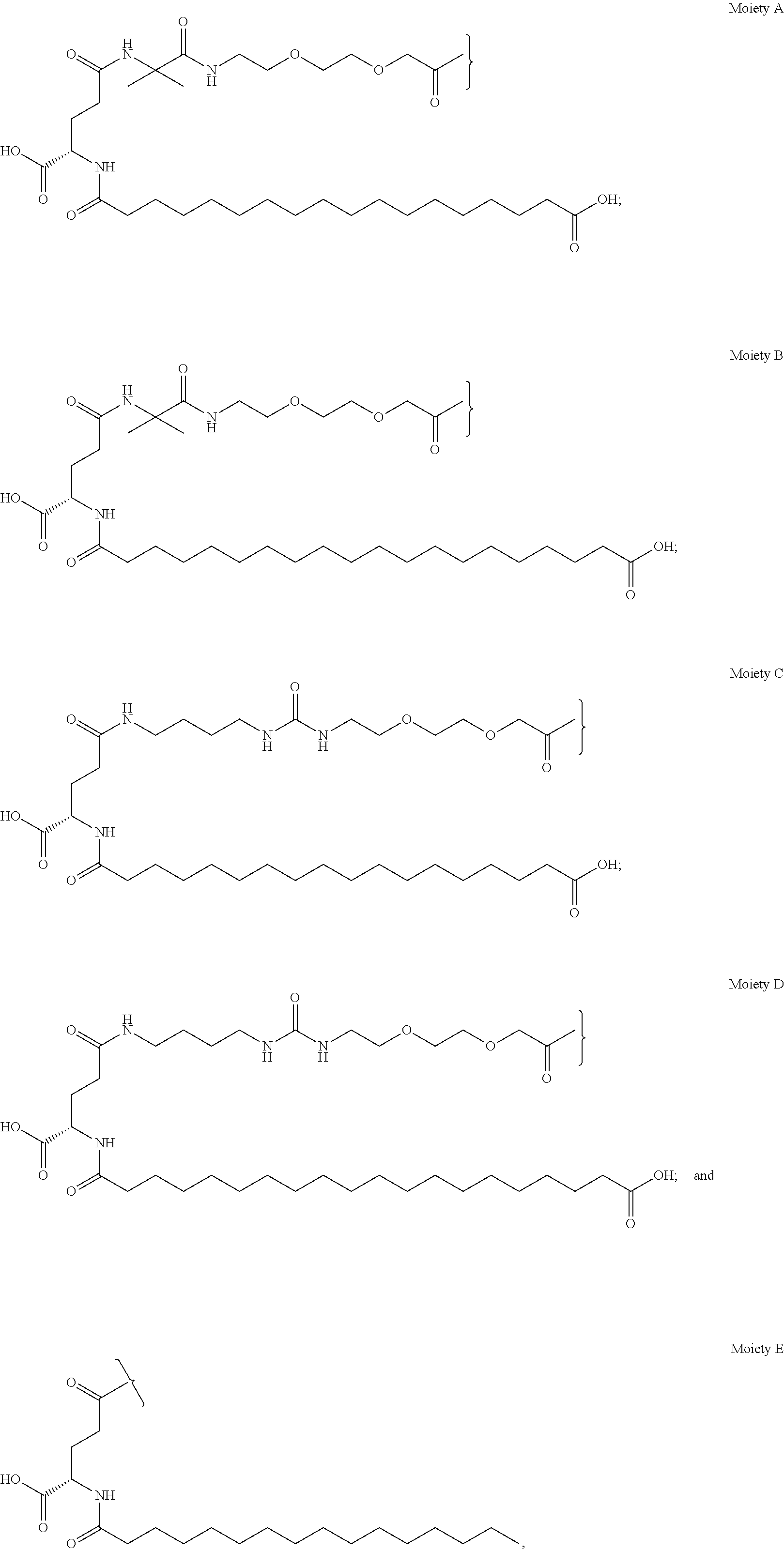 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O ; CCCCCCCCCCCCCCCC(=O)N[C@@H](CCC(C)=O)C(=O)O]

with a provisio that the polypeptide is not SEQ ID NO: 6.

In another aspect, the present disclosure provides a polypeptide or a pharmaceutically acceptable salt thereof comprising the amino acid sequence:

(SEQ ID NO: 4)
H-Aib-Q-G-T-F-T-S-D-Y-S-X12-X13-L-D-E-K-K-A-X20-
E-F-V-E-W-L-L-E-G-G-P-S-S-G

wherein:
   X12 is K or I;
   X13 is Y or nor-V; and
   X20 is K;
wherein the acid group of the C-terminal amino acid is a free carboxylic acid group or is amidated as C-terminal primary amide; and
wherein the side chain amino (ε amino) group of K at position X20 is acylated with a moiety of the formula selected from:

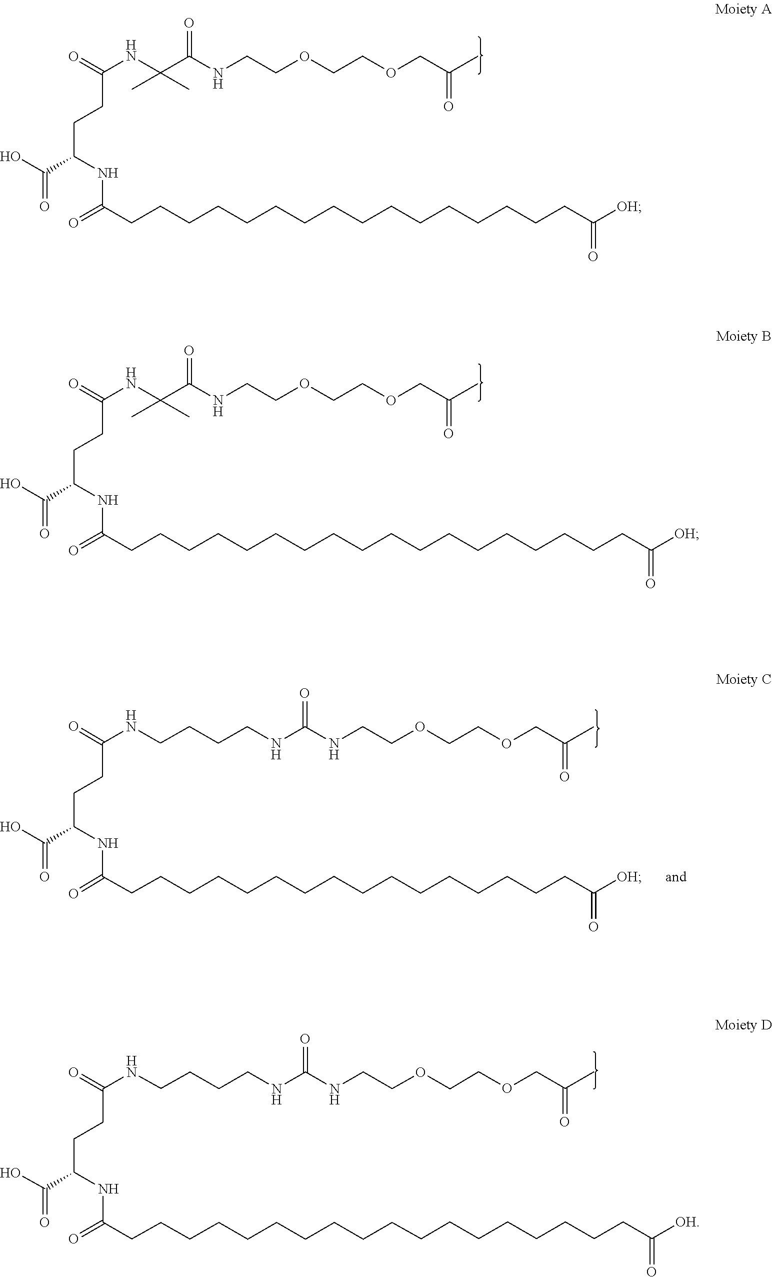 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; "and" ; CC(=O)COCCOCCNC(=O)NCCCCNC(=O)CC[C@H](NC(=O)CCCCCCCCCCCCCCCCCCC(=O)O)C(=O)O]

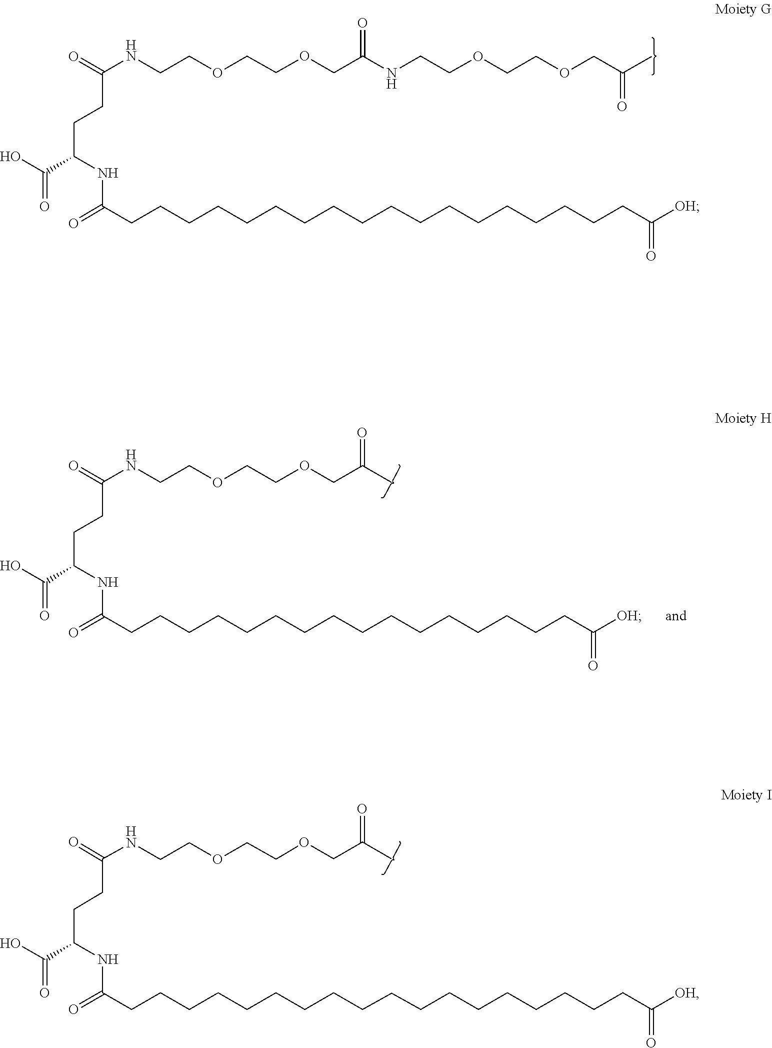 [SMILES: CC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)CC[C@H](NC(=O)CCCCCCCCCCCCCCCCC(=O)O)C(=O)O ; "and" ; CC(=O)COCCOCCNC(=O)CC[C@H](NC(=O)CCCCCCCCCCCCCCCCCCC(=O)O)C(=O)O]

In another aspect, the present disclosure provides a polypeptide or a pharmaceutically acceptable salt thereof comprising the amino acid sequence:

(SEQ ID NO: 5)
H-(DSer)-Q-G-T-F-T-S-D-X10-S-K-Y-L-D-A-R-A-A-Q-D-
F-V-Q-W-L-L-D-T

wherein X10 is K;
wherein the acid group of the C-terminal amino acid is a free carboxylic acid group or is amidated as C-terminal primary amide; and
wherein the side chain amino (ε amino) group of K at position X10 is acylated with a moiety of the formula selected from:

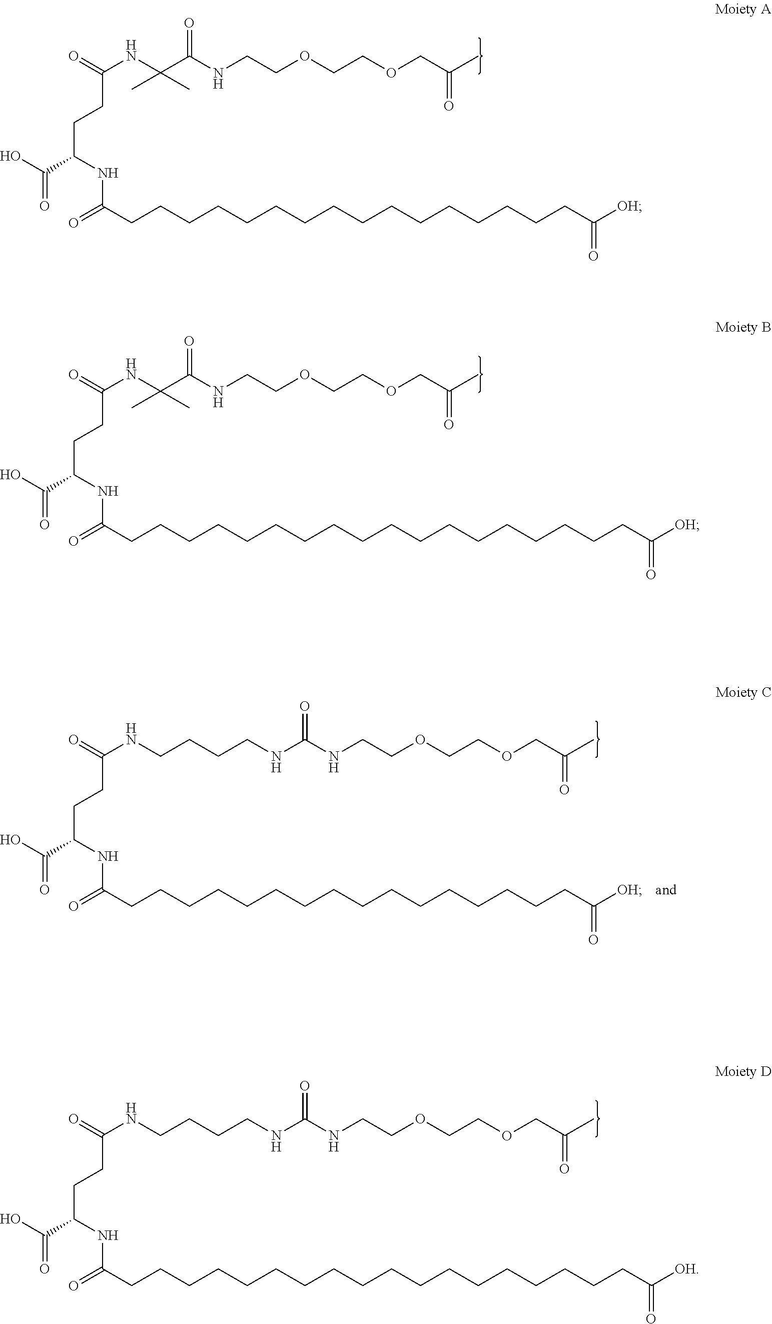 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

In one aspect, the present disclosure provides a polypeptide which is selected from the group consisting of:

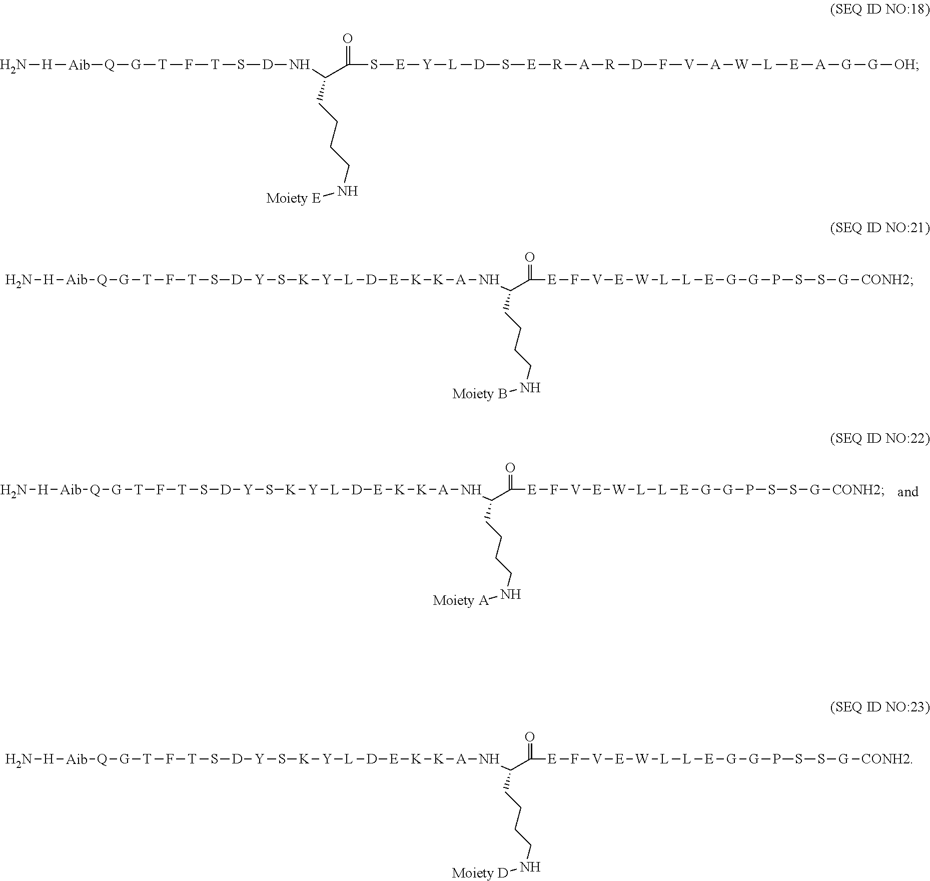

In another aspect, the present disclosure relates to an incretin analog comprising:
a peptide residue having the sequence X¹-Gln-Gly-Thr-Phe-Thr-Ser-Asp-Lys (SEQ ID NO:29), wherein X¹ represents Aib or Ser(OMe), and wherein the lysine comprises a fatty acid protracting group attached to the lysine ε-nitrogen; and
a Gly-Gly-OH peptide residue indirectly attached to the carboxy of the lysine.

In another aspect, the present disclosure provides an incretin analog comprising:
a peptide residue having the sequence X¹-Gln-Gly-Thr-Phe-Thr-Ser-Asp-Lys (SEQ ID NO:29), wherein X¹ represents Aib or Ser(OMe), and wherein the lysine comprises a group of formula (I) attached to the lysine ε-nitrogen,

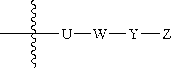

wherein:
   U is absent or represents —C(O)—CH₂—O—(CH₂)₂—O—(CH₂)₂—NH—}, wherein } is point of attachment to W;
   W represents:
      —C(O)—CH₂—O—(CH₂)₂—O—(CH₂)₂—NH—],
      —C(O)—NH—(CH₂)₃₋₄—NH—], —C(O)—C(CH₃)₂—NH—], or

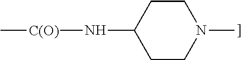

wherein ] is point of attachment to Y;
Y is absent or represents —C(O)—(CH₂)₂—CH(CO₂H)NH—or —C(O)CH((CH₂)ₓCO₂H)NH—, wherein x is 1, 2 or 3, and wherein — is point of attachment to Z; and
Z represents —C(O)—(CH₂)ₙ—COOH or —C(O)—(CH₂)ₙ—CH₃, wherein n is an integer from 14-20; and
a Gly-Gly-OH peptide residue indirectly attached to the carboxy of the lysine.

In another aspect, the present disclosure provides an incretin analog comprising:
a peptide residue having the sequence Aib-Gln-Gly-Thr-Phe-Thr-Ser-Asp (SEQ ID NO:30);
a lysine residue indirectly attached to the carboxy of the Aib-Gln-Gly-Thr-Phe-Thr-Ser-Asp (SEQ ID NO:30) residue, wherein the lysine comprises a group of formula (I) attached to the lysine ε-nitrogen;

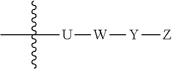

wherein:
   U is absent or represents —C(O)—CH₂—O—(CH₂)₂—O—(CH₂)₂—NH—}, wherein } is point of attachment to W;
   W represents:
      —C(O)—CH₂—O—(CH₂)₂—O—(CH₂)₂—NH—], or
      —C(O)—NH—(CH₂)₃₋₄—NH—], —C(O)—C(CH₃)₂—NH—],
      wherein ] is point of attachment to Y;
   Y is absent or represents —C(O)—(CH₂)₂—CH(CO₂H)NH—or —C(O)CH((CH₂)ₓCO₂H)NH—, wherein x is 1, 2 or 3, and wherein — is point of attachment to Z; and
   Z represents —C(O)—(CH₂)ₙ—COOH or —C(O)—(CH₂)ₙ—CH₃, wherein n is an integer from 14-20; and
a Gly-Gly-Pro-Ser-Ser-Gly-CONH₂ peptide residue indirectly attached to the carboxy of the lysine.

In another aspect, the present disclosure relates to pharmaceutical compositions comprising a pharmaceutically acceptable carrier and an incretin analog or a polypeptide as described herein.

In another aspect, the present disclosure relates to a method of treating obesity, Type 2 diabetes mellitus (T2DM), metabolic syndrome, metabolic dysfunction-associated steatotic liver disease (MASLD), metabolic dysfunction-associated steatohepatitis (MASH), neurodegenerative disorders, fibrosis, cardiovascular risks, and/or hyperlipidemia/dyslipidemia, the method comprising administering to a patient in need of such treatment an incretin analog or a polypeptide as described herein.

### DETAILED DESCRIPTION OF THE INVENTION

### Abbreviations

Aib: 2-aminoisobutyric acid
DIPEA: N,N′-di-isopropylethylamine
HOBt: 1-hydroxybenztriazole
DIPC: N,N′-di-isopropylcarbodiimide
THF: tetrahydrofuran
DCM: dichloromethane
Fmoc: fluorenylmethyloxycarbonyl
HOSu: N-hydroxysuccinimide
DCC: dicyclohexyl carbodiimide
DMAc: dimethylacetamide
IBCF: isobutyl chloroformate
NMM: N-methylmorpholine
DIC: diisopropylcarbodiimide

### DEFINITIONS

“Pharmaceutically acceptable salts” according to the present disclosure include acid addition salts formed with either organic or inorganic acids. Suitable pharmaceutically acceptable salts of the compounds of the present disclosure include acid addition salts which may be salts of inorganic acids such as hydrochloric acid, hydrobromic acid, phosphoric acid, or the like, or of organic acids such as acetic acid, benzenesulfonic acid, methanesulfonic acid, benzoic acid, citric acid, lactic acid, fumaric acid, succinic acid, adipic acid, pimelic acid, suberic acid, azelaic acid, malic acid, tartaric acid, amino acids (e.g., glutamic acid or aspartic acid), or the like. The pharmaceutically acceptable acid addition salts of the compounds of the present disclosure include salts formed with the addition of one or more equivalents of acids, such as monohydrochloride or dihydrochloride salts. Salts can be prepared by any process under the purview of an ordinary person skilled in the art. See, Berge et al., J. Pharm. Sci., 1977, 66, 1-19; and “Handbook of Pharmaceutical Salts: Properties, Selection, and Use,” edited by Stahl et al., Verlag Helv. Chim. Acta, Zurich, Switzerland, and Wiley-VCH, Weinheim, Germany, 2002.

The term “effective amount or amount effective” as used herein refers to an amount of a compound which is sufficient, upon single or multiple dose administration(s) to a subject, in curing, alleviating, relieving, or partially addressing the clinical manifestation of a given disease or state and its complications beyond that expected in the absence of such treatment. Thus, the result can be reduction and/or alleviation of the signs, symptoms, or causes of a disease, or any other desired alteration of a biological system. It is understood that “a therapeutically effective amount” can vary from subject to subject depending on age, weight, general condition of the subject, the condition being treated, the severity of the condition being treated, and the judgment of the prescribing physician.

The amino “Aib” as used herein can be represented by structure:

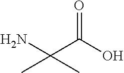

and can also be defined by the chemical name of “2-aminoisobutyric acid.”

The amino acid “S(OMe)” or “Ser(OMe)” as used herein can be represented by structure:

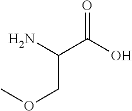

and can also be defined by the chemical name of “serine methyl ether.” The terms L-Ser(OMe) and D-Ser(OMe) refer to “L” and “D” isomers of Ser(OMe), respectively.

The amino acid “nor-V”, “nor-Val” or “norvaline” as used herein can be represented by structure:

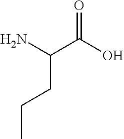

and can also be defined by the chemical name of “2-aminopentanoic acid.” The terms L-norvaline and D-norvaline refer to “L” and “D” isomers of norvaline, respectively.

The amino acid “nor-L”, “nor-Leu” or “norleucine” as used herein can be represented by structure:

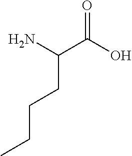

and can also be defined by the chemical name of “2-aminohexanoic acid.” The terms L-norleucine and D-norleucine refer to “L” and “D” isomers of norleucine, respectively.

The amino acid “αMe-L”, “αMe-Leu” or “αMe-leucine” as used herein can be represented by structure:

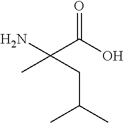

and can also be defined by the chemical name of “2-amino-2,4-dimethylpentanoic acid.” The terms L-α-Me-Leucine and D-α-Me-Leucine refer to “L” and “D” isomers of α-Me-Leucine, respectively.

As described herein, the present disclosure provides stable, long-acting GLP-1 mono, GLP-1/GIP dual, and/or GLP-1/GCG dual receptor agonists which may be useful for treating T2DM, hyperlipidemia/dyslipidemia, metabolic syndromes, non-alcoholic fatty liver diseases (NAFLD), non-alcoholic steatohepatitis (NASH), neurodegenerative disorders, fibrosis, and/or obesity, and reducing cardiovascular risks.

In one aspect, the present disclosure provides a polypeptide or a pharmaceutically acceptable salt thereof comprising the amino acid sequence:

(SEQ ID NO: 1)
X1-X2-X3-G-T-F-T-S-D-X10-S-X12-X13-L-D-X16-X17-
X18-X19-X20-X21-F-X23-X24-X25-L-X27-X28-X29-X30-
X31-X32-X33-X34-X35-X36-X37-X38-X39

wherein:
   X1 is H;
   X2 is D-Ser(OMe), Aib or D-S;
   X3 is Q;
   X10 is K or Y;
   X12 is E, K or I;
   X13 is Y, S(OMe), nor-V, nor-L, or αMe-L;
   X16 is S, E or A;
   X17 is E, R or K;
   X18 is, K or A;
   X19 is A;
   X20 is R, Q or K;
   X21 is D or E;
   X23 is V or I;
   X24 is A, Q or E;
   X25 is W;
   X27 is E or L;
   X28 is A, D or E;
   X29 is G or T;
   X30 is absent or G;
   X31 is absent or P;
   X32 is absent or S;
   X33 is absent or S;
   X34 is absent or G
   X35 is absent;
   X36 is absent;
   X37 is absent;
   X38 is absent; and
   X39 is absent;
wherein the acid group of the C-terminal amino acid is a free carboxylic acid group or is amidated as C-terminal primary amide;
with a proviso that at least one of X10 and X20 is K, and further provided at least one of said K comprises a side chain amino (ε amino) group acylated with a moiety of the formula selected from:

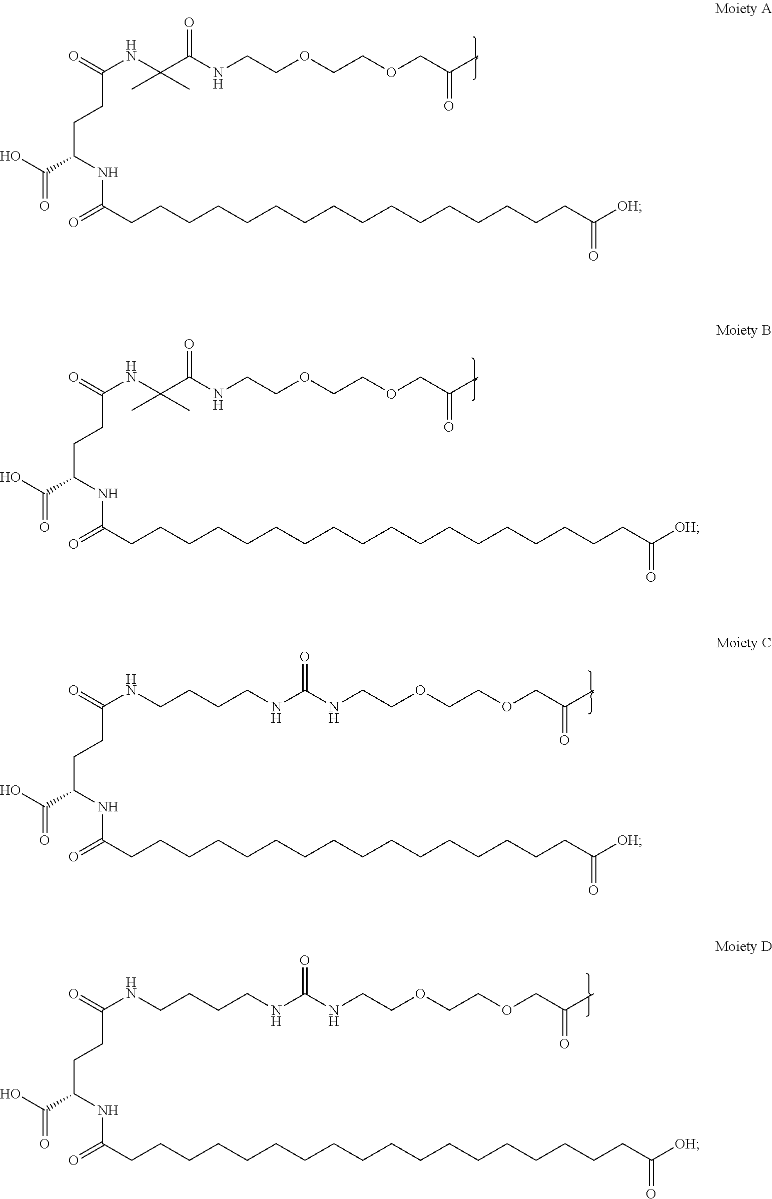 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

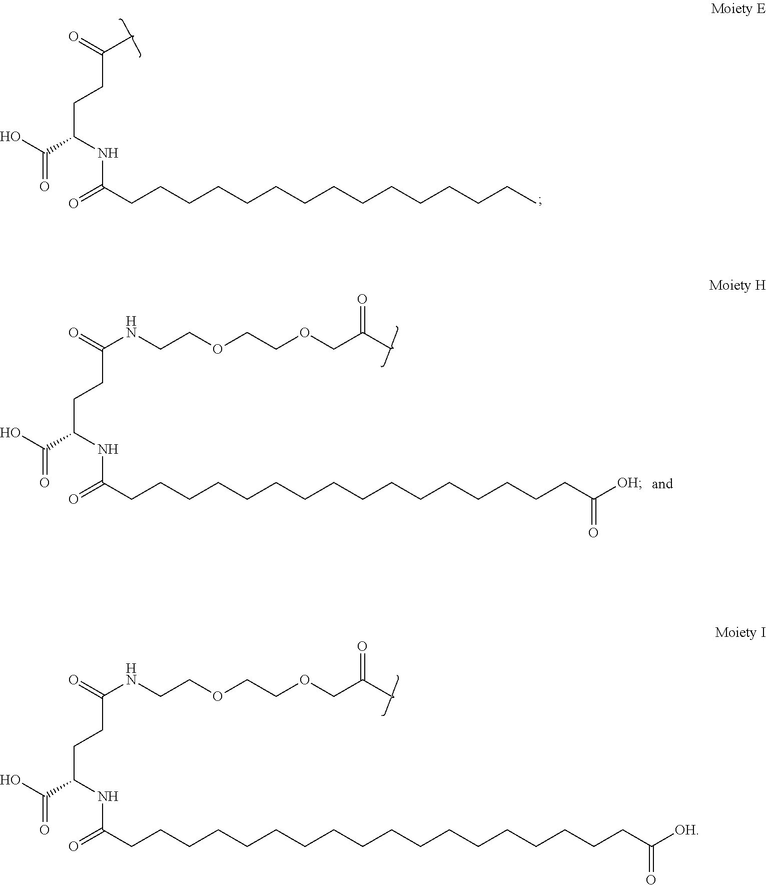 [SMILES: CCCCCCCCCCCCCCCC(=O)N[C@@H](CCC(C)=O)C(=O)O ; CC(=O)COCCOCCNC(=O)CC[C@H](NC(=O)CCCCCCCCCCCCCCCCC(=O)O)C(=O)O ; CC(=O)COCCOCCNC(=O)CC[C@H](NC(=O)CCCCCCCCCCCCCCCCCCC(=O)O)C(=O)O]

In one embodiment, the polypeptide of SEQ ID NO: 1 comprises the following sequence:
X2 is D-Ser(OMe) or Aib;
X10 is K;
X12 is E;
X13 is Y;
X16 is S;
X17 is E;
X18 is R;
X20 is R;
X21 is D;
X24 is A;
X27 is E;
X28 is A;
X29 is G;
X30 is G; and
X31, X32, X33 and X34 are absent;
wherein the side chain amino (ε amino) group of K at position X10 is acylated with a moiety of the formula selected from:

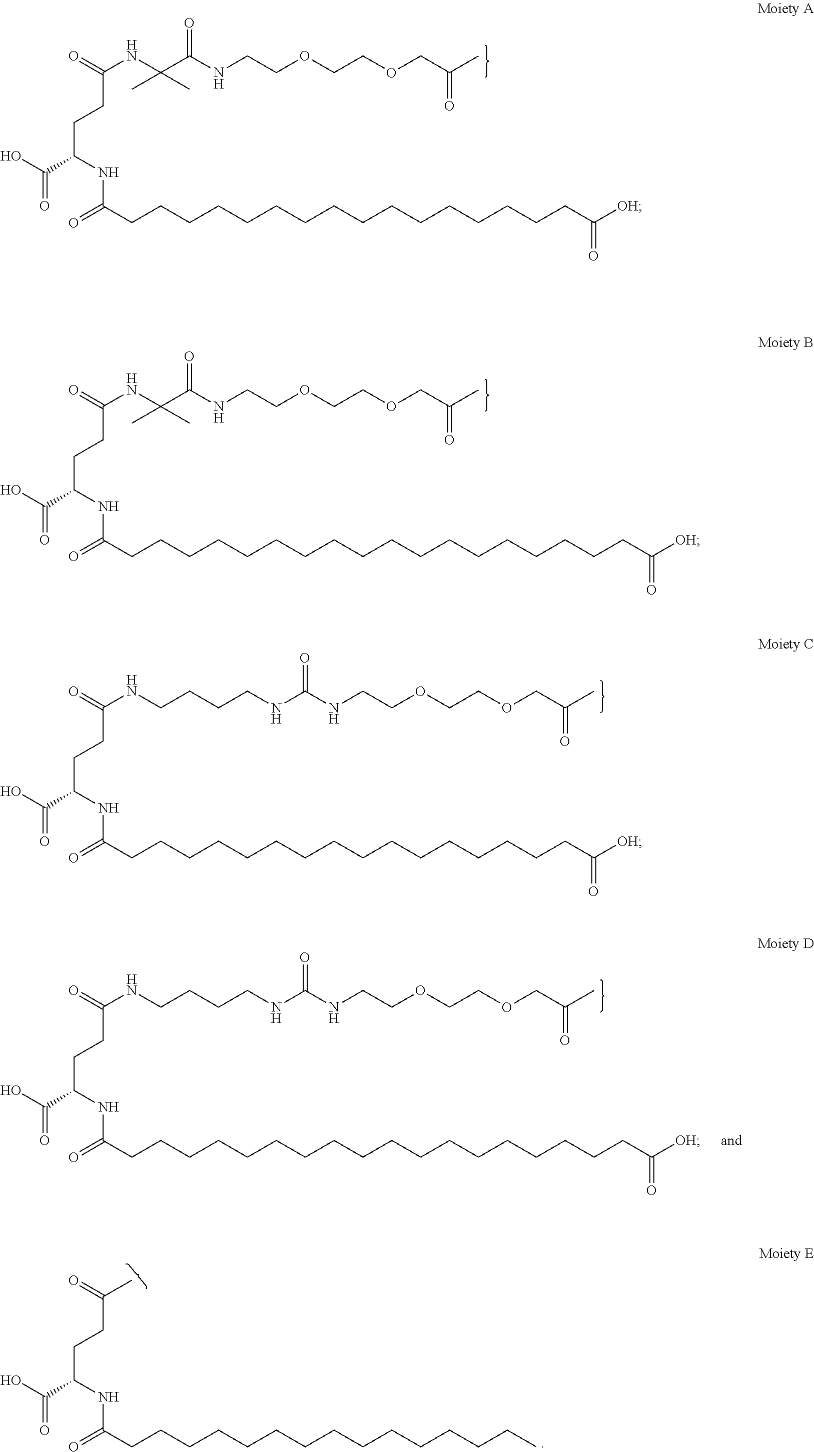 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O ; CCCCCCCCCCCCCCCC(=O)N[C@@H](CCC(C)=O)C(=O)O]

In another aspect, the present disclosure provides a polypeptide or a pharmaceutically acceptable salt thereof comprising the amino acid sequence:

(SEQ ID NO: 2)
X1-X2-X3-G-T-F-T-S-D-X10-S-X12-X13-L-D-X16-X17-
X18-X19-X20-X21-F-X23-X24-X25-L-X27-X28-X29-X30-
X31-X32-X33-X34-X35-X36-X37-X38-X39

wherein:
   X1 is H;
   X2 is S, D-Ser(OMe), Aib, or D-S;
   X3 is Q;
   X10 is K or Y;
   X12 is E, K or I;
   X13 is Y, S(OMe), nor-V, nor-L, or αMe-L;
   X16 is S, E or A;
   X17 is E, R or K;
   X18 is R, K or A;
   X19 is A;
   X20 is R, Q or K,
   X21 is D or E;
   X23 is V;
   X24 is A, Q or E;
   X25 is W;
   X27 is E or L;
   X28 is A, D or E;
   X29 is G or T;
   X30 is absent or G;
   X31 is absent or P;
   X32 is absent or S;
   X33 is absent or S;
   X34 is absent or G;
   X35 is absent;
   X36 is absent;
   X37 is absent;
   X38 is absent; and
   X39 is absent;
wherein the acid group of the C-terminal amino acid is a free carboxylic acid group or is amidated as C-terminal primary amide;
with a proviso that at least one of X10 and X20 is K, and further provided that at least one of said K comprises a side chain amino (ε amino) group acylated with a moiety of the formula selected from:

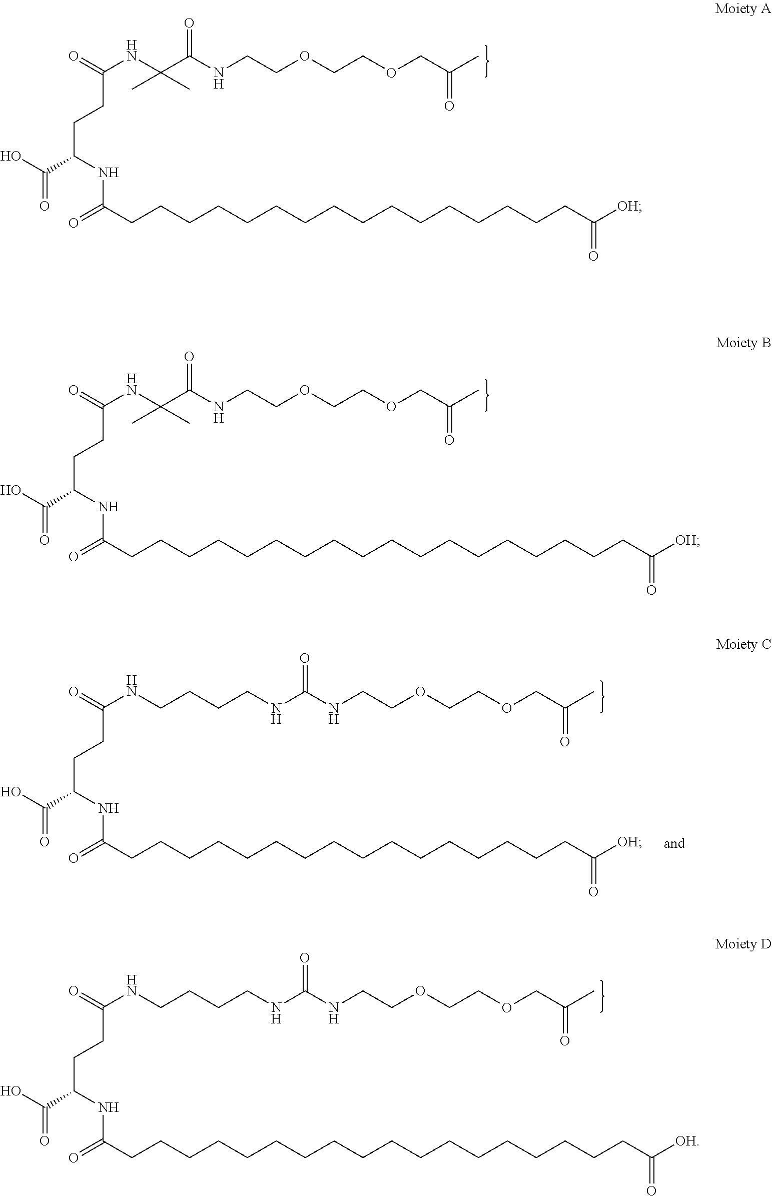 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

In one embodiment, the polypeptide of SEQ ID NO: 1 or SEQ ID NO: 2 comprises the following sequence:
X2 is D-S,
X10 is K,
X12 is K,
X13 is Y;
X16 is A,
X17 is R,
X18 is A,
X20 is Q;
X21 is D,
X24 is Q;
X27 is L;
X28 is D;
X29 is T; and
X31, X32, X33 and X34 are absent;
wherein the side chain amino (ε amino) group of K at position X10 is acylated with a moiety of the formula selected from:

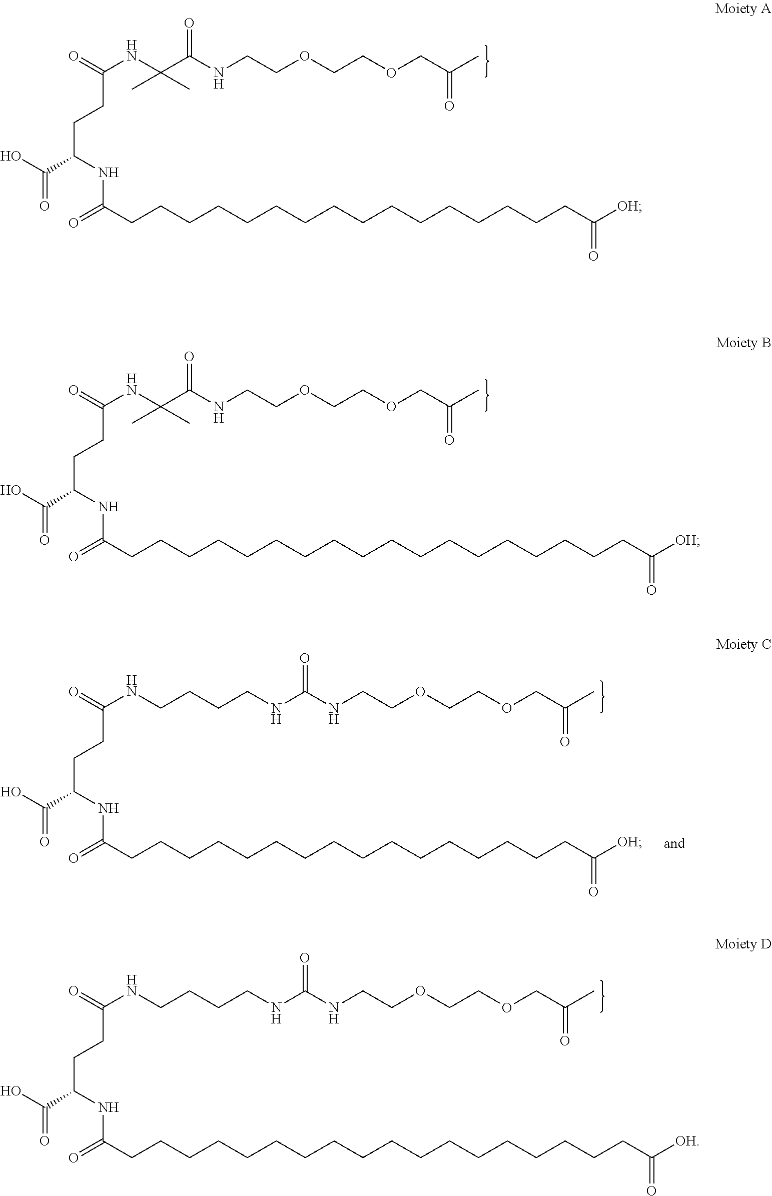 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

In another embodiment, the polypeptide of SEQ ID NO: 1 or SEQ ID NO: 2 comprises the following sequence:
X2 is Ab;
X10 is Y;
X12 is K or I;
X13 is Y, nor-V, nor-L, or αMe-L;
X16 is E;
X17 is K;
X18 is K;
X20 is K;
X21 is E;
X24 is E;
X27 is L;
X28 is E;
X29 is G;
X30 is G
X31 is P;
X32 is S;
X33 is S; and
X34 is G;
wherein, the side chain amino (ε amino) group of K at position X20 is acylated with a moiety of the formula selected from:

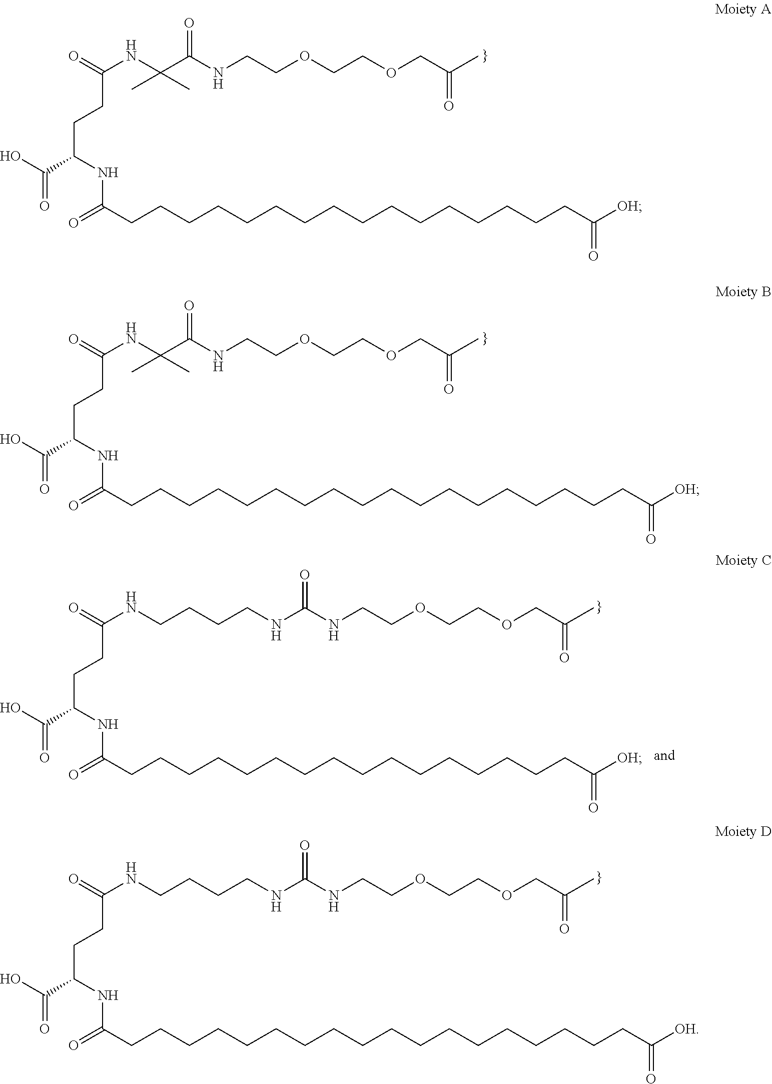 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

In another aspect, the present disclosure provides a polypeptide or a pharmaceutically acceptable salt thereof comprising the amino acid sequence:

(SEQ ID NO: 3)
H-X2-Q-G-T-F-T-S-D-X10-S-E-Y-L-D-S-E-R-A-R-D-F-V-
A-W-L-E-A-G-G

wherein:
   X2 is S, D-S(OMe) or Aib; and
   X10 is K;
wherein the acid group of the C-terminal amino acid is a free carboxylic acid group or is amidated as C-terminal primary amide; and
wherein the side chain amino (ε amino) group of K at position X10 is acylated with a moiety of the formula selected from:

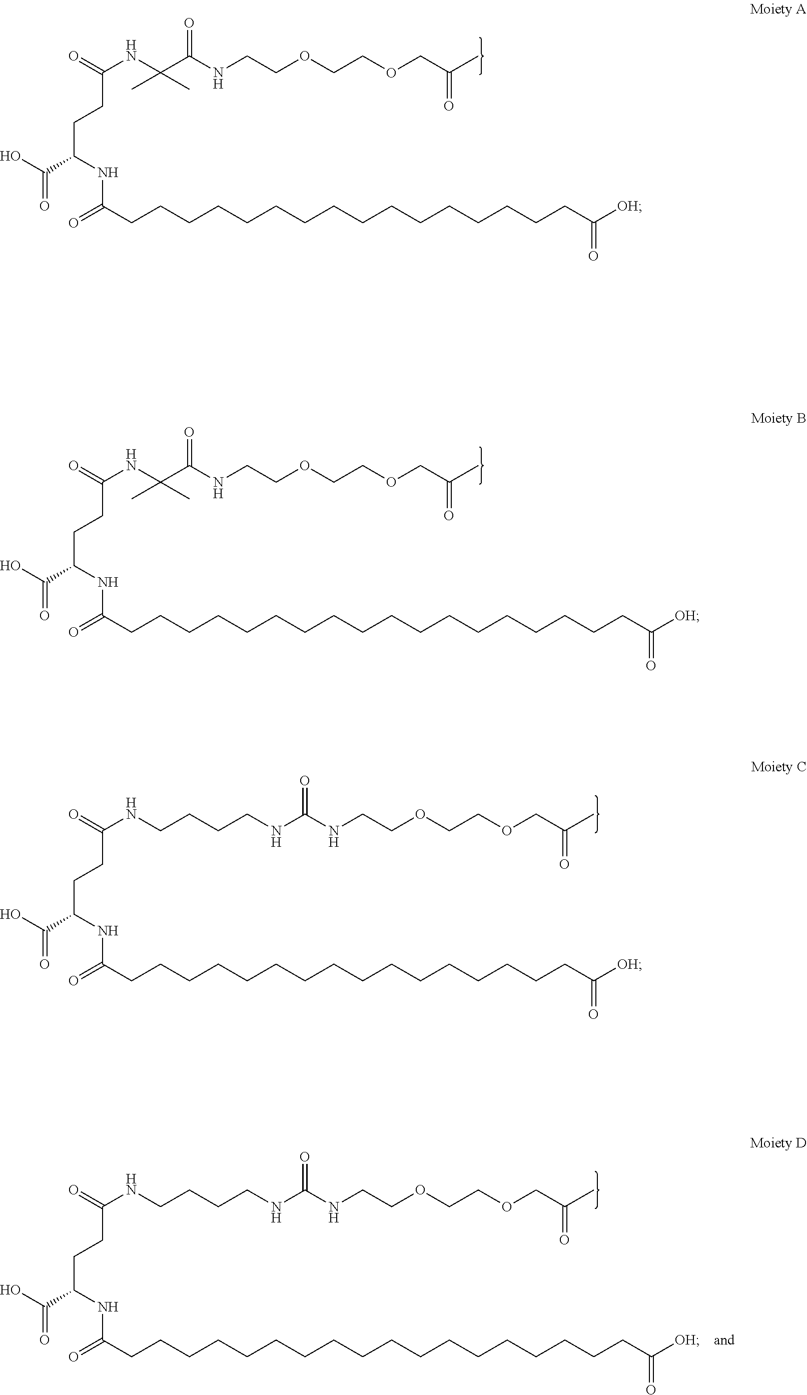 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

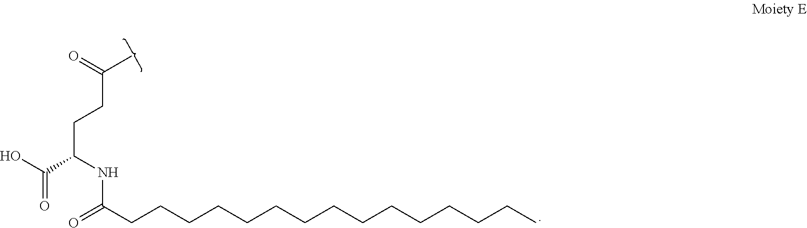 [SMILES: CCCCCCCCCCCCCCCC(=O)N[C@@H](CCC(C)=O)C(=O)O]

with a proviso that polypeptide is not SEQ ID NO: 6.

In one embodiment of the polypeptide of SEQ ID NO: 3, X2 is S;
wherein the side chain amino (ε amino) group of K at position X10 is acylated with a moiety of the formula selected from:

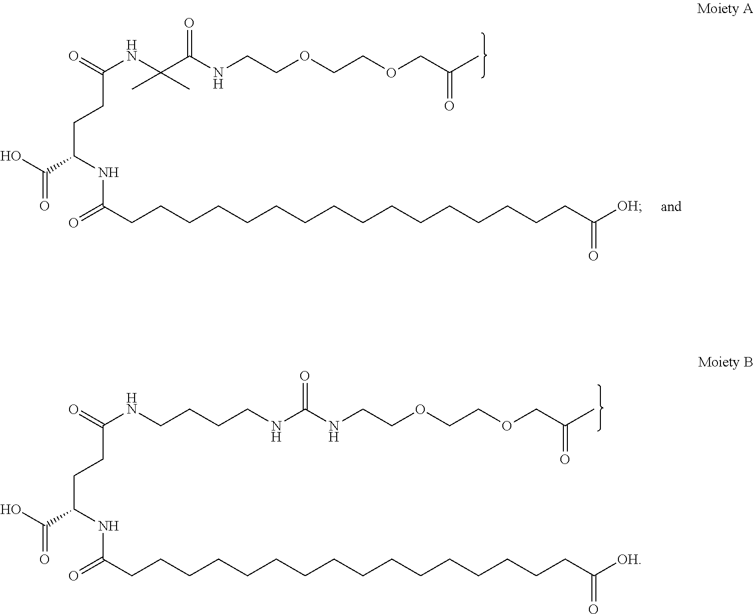 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O]

In another embodiment of the polypeptide of SEQ ID NO: 3,
X2 is D-Ser(OMe);
wherein the side chain amino (ε amino) group of K at position X10 is acylated with a moiety of the formula:

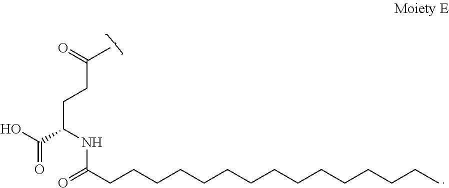 [SMILES: CCCCCCCCCCCCCCCC(=O)N[C@@H](CCC(C)=O)C(=O)O]

In another embodiment of the polypeptide of SEQ ID NO: 3, X2 is Aib;
wherein the side chain amino (ε amino) group of K at position X10 is acylated with a moiety of the formula selected from:

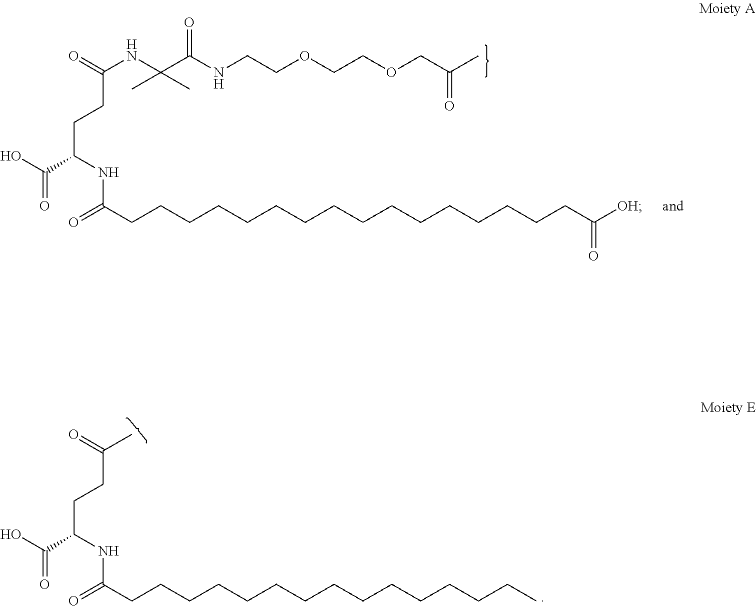 [SMILES: CC(=O)COCCOCCNC(=O)C(C)(C)NC(=O)CC[C@H](NC(=O)CCCCCCCCCCCCCCCCC(=O)O)C(=O)O ; CCCCCCCCCCCCCCCC(=O)N[C@@H](CCC(C)=O)C(=O)O]

In another aspect, the present disclosure provides a polypeptide or a pharmaceutically acceptable salt thereof comprising the amino acid sequence:

(SEQ ID NO: 4)
H-Aib-Q-G-T-F-T-S-D-Y-S-X12-X13-L-D-E-K-K-A-X20-
E-F-V-E-W-L-L-E-G-G-P-S-S-G

wherein:
   X12 is K or I;
   X13 is Y or nor-V; and
   X20 is K;
wherein the acid group of the C-terminal amino acid is a free carboxylic acid group or is amidated as C-terminal primary amide; and
wherein the side chain amino (ε amino) group of K at position X20 is acylated with a moiety of the formula selected from:

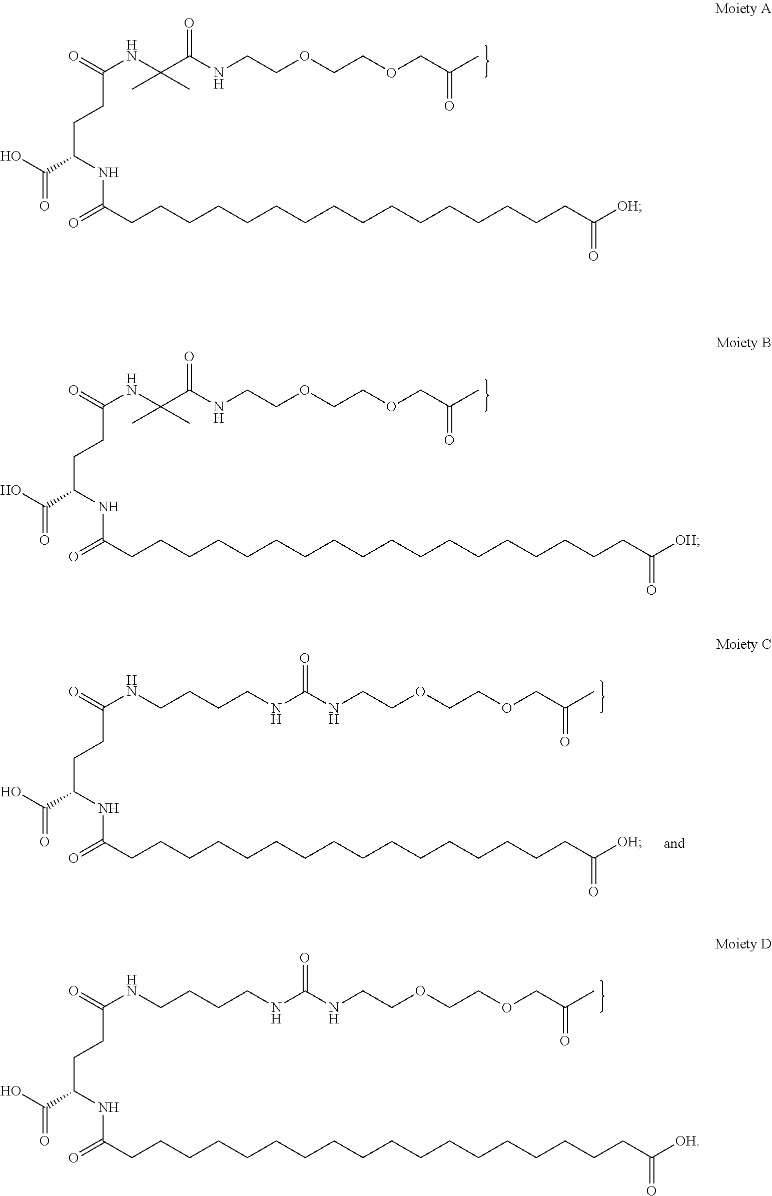 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

In one embodiment of, the polypeptide of SEQ ID NO: 4,
X12 is K; and
X13 is Y;
wherein the side chain amino (ε amino) group of K at position X20 is acylated with a moiety of the formula selected from:

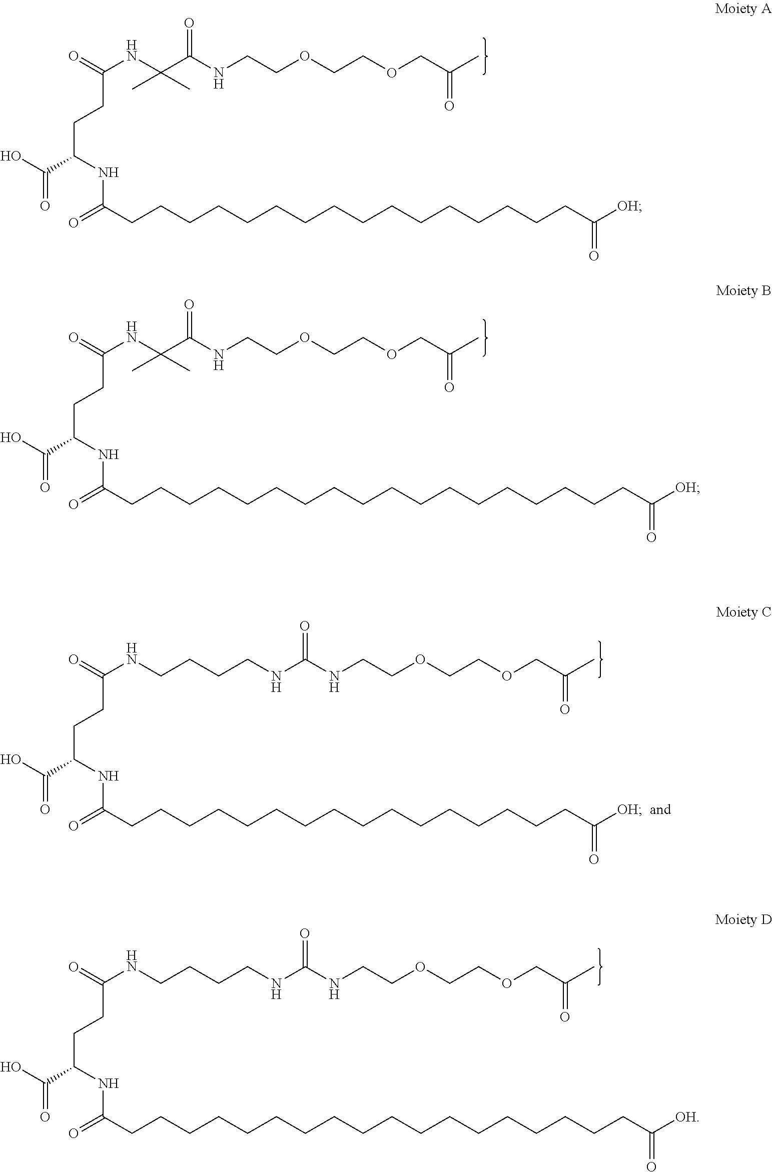

In another embodiment of the polypeptide of SEQ ID NO: 4,
X12 is I; and
X13 is nor-V;
wherein the side chain amino (ε amino) group of K at position X20 is acylated with a moiety of the formula:

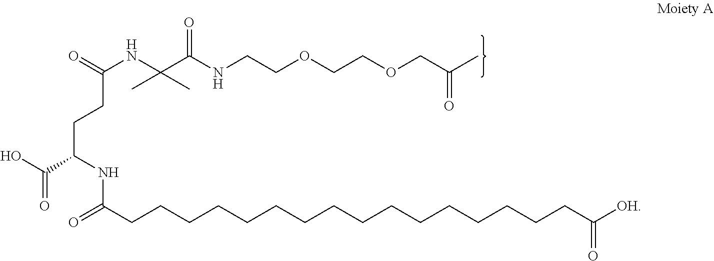 [SMILES: CC(=O)COCCOCCNC(=O)C(C)(C)NC(=O)CC[C@H](NC(=O)CCCCCCCCCCCCCCCCC(=O)O)C(=O)O]

In another embodiment of the polypeptide of SEQ ID NO: 4,
X12 is K; and
X13 is nor-V;
wherein the side chain amino (ε amino) group of K at position X20 is acylated with a moiety of the formula:

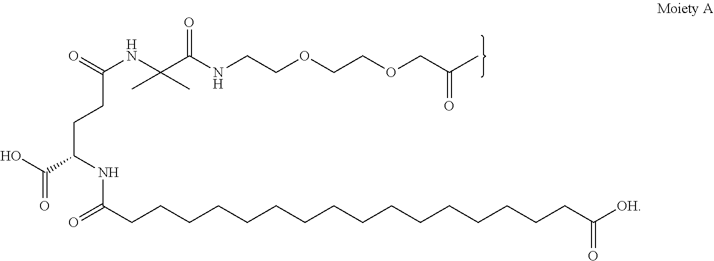 [SMILES: CC(=O)COCCOCCNC(=O)C(C)(C)NC(=O)CC[C@H](NC(=O)CCCCCCCCCCCCCCCCC(=O)O)C(=O)O]

In another aspect, the present disclosure provides a polypeptide or a pharmaceutically acceptable salt thereof corn rising the amino acid sequence:

(SEQ ID NO: 5)
H-(DSer)-Q-G-T-F-T-S-D-X10-S-K-Y-L-D-A-R-A-A-Q-D-
F-V-Q-W-L-L-D-T

wherein X10 is K;
wherein the acid group of the C-terminal amino acid is a free carboxylic acid group or is amidated as C-terminal primary amide; and
wherein the side chain amino (ε amino) group of K at position X10 is acylated with a moiety of the formula selected from:

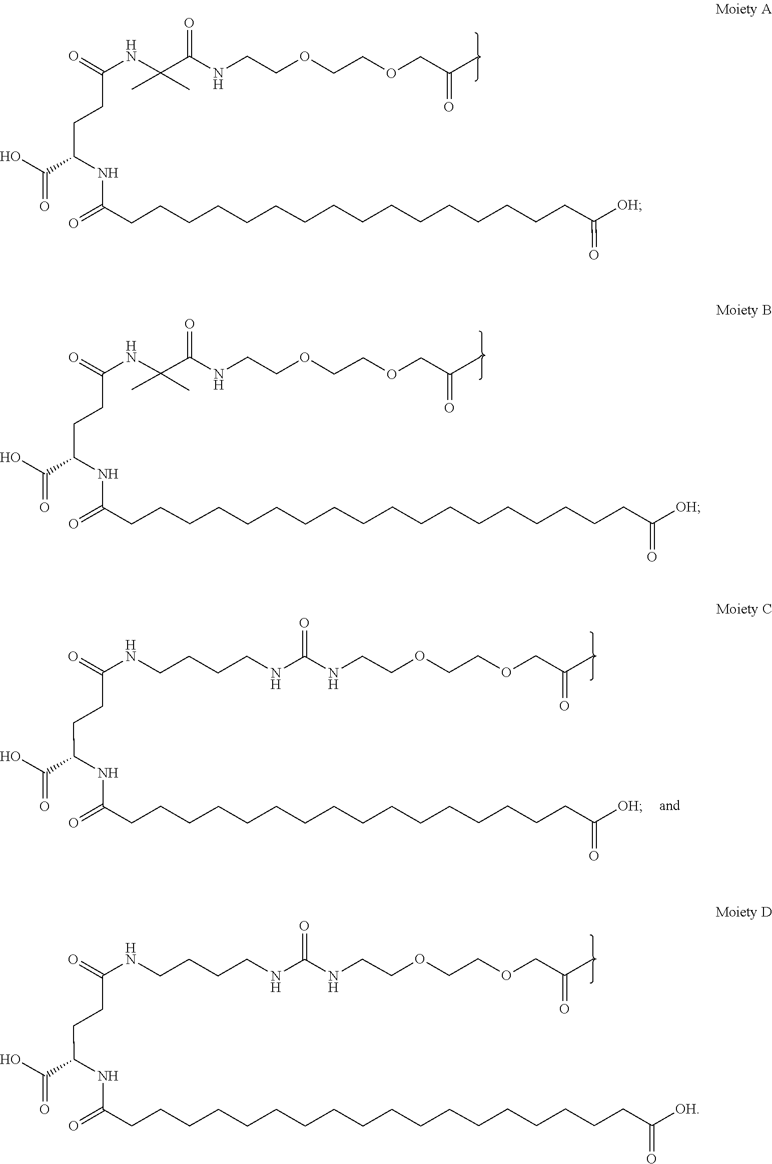 [SMILES: CC(=O)COCCOCCNC(=O)C(C)(C)NC(=O)CC[C@H](NC(=O)CCCCCCCCCCCCCCCCC(=O)O)C(=O)O ; CCC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

In another aspect, the present disclosure provides an incretin analog comprising:
a peptide residue having the sequence X¹-Gln-Gly-Thr-Phe-Thr-Ser-Asp-Lys (SEQ ID NO:29), wherein X¹ represents Aib or Ser(OMe), and wherein the lysine comprises a fatty acid protracting group attached to the lysine ε-nitrogen; and
a Gly-Gly-OH peptide residue indirectly attached to the carboxy of the lysine.

In one embodiment, the incretin analog has lysine attached to the Gly-Gly-OH residue by a peptide residue comprising 18 amino acids.

In another aspect, the present disclosure provides an incretin analog comprising:
a peptide residue having the sequence X¹-Gln-Gly-Thr-Phe-Thr-Ser-Asp-Lys (SEQ ID NO:29), wherein X¹ represents Aib or Ser(OMe), and wherein the lysine comprises a group of formula (I) attached to the lysine ε-nitrogen,

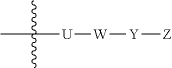

wherein:
   U is absent or represents —C(O)—CH₂—O—(CH₂)₂—O—(CH₂)₂—NH—}, wherein } is point of attachment to W;
   W represents:
      —C(O)—CH₂—O—(CH₂)₂—O—(CH₂)₂—NH—],
      —C(O)—NH—(CH₂)₃₋₄—NH—], —C(O)—C(CH₃)₂—NH—], or

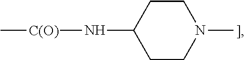

wherein ] is point of attachment to Y;
Y is absent or represents —C(O)—(CH₂)₂—CH(CO₂H)NH—or —C(O)CH((CH₂)ₓCO₂H)NH—, wherein x is 1, 2 or 3, and wherein — is point of attachment to Z; and
Z represents —C(O)—(CH₂)ₙ—COOH or —C(O)—(CH₂)ₙ—CH₃, wherein n is an integer from 14-20; and
a Gly-Gly-OH peptide residue indirectly attached to the carboxy of the lysine.

In one embodiment, the incretin analog has lysine attached to the Gly-Gly-OH residue by a peptide residue comprising 18 amino acids.

In another aspect, the present disclosure provides an incretin analog comprising:
a peptide residue having the sequence Aib-Gln-Gly-Thr-Phe-Thr-Ser-Asp (SEQ ID NO:30);
a lysine residue indirectly attached to the carboxy of the Aib-Gln-Gly-Thr-Phe-Thr-Ser-Asp (SEQ ID NO:30) residue, wherein the lysine comprises a group of formula (I) attached to the lysine E-nitrogen;

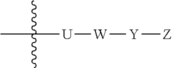

wherein:
   U is absent or represents —C(O)—CH₂—O—(CH₂)₂—O—(CH₂)₂—NH—}, wherein } is point of attachment to W;
   W represents
      —C(O)—CH₂—O—(CH₂)₂—O—(CH₂)₂—NH—], or
      —C(O)—NH—(CH₂)₃₋₄—NH—], —C(O)—C(CH₃)₂—NH—],
      wherein ] is point of attachment to Y;
   Y is absent or represents —C(O)—(CH₂)₂—CH(CO₂H)NH—or —C(O)CH((CH₂)ₓCO₂H)NH—, wherein x is 1, 2 or 3, and wherein — is point of attachment to Z; and
   Z represents —C(O)—(CH₂)ₙ—COOH or —C(O)—(CH₂)ₙ—CH₃, wherein n is an integer from 14-20; and
a Gly-Gly-Pro-Ser-Ser-Gly-CONH₂ peptide residue indirectly attached to the carboxy of the lysine.

In one embodiment, the incretin analog has lysine attached to the Aib-Gln-Gly-Thr-Phe-Thr-Ser-Asp (SEQ ID NO:30) residue by a peptide residue comprising 10 amino acids.

In another aspect, the polypeptide as described herein excludes the polypeptides of SEQ ID NO: 6, SEQ ID NO: 7, SEQ ID NO: 8, and SEQ ID NO: 27.

In another aspect, the present disclosure provides a polypeptide or pharmaceutically acceptable salt thereof comprising an amino acid sequence selected from the group consisting of:

i.)
(SEQ ID NO: 9)
HSQGTFTSDK*SEYLDSERARDFVAWLEAGG-OH;
ii.)
(SEQ ID NO: 10)
H-(DS(OMe))-QGTFTSDK*SEYLDSERARDFVAWLEAGG-OH;
iii.)
(SEQ ID NO: 11)
H-Aib-SQGTFTSDK*SEYLDSERARDFVAWLEAGG-OH;
iv.)
(SEQ ID NO: 12)
H-(D-S)-QGTFTSDK*SKYLDARAAQDFVQWLLDT-NH₂;
v.)
(SEQ ID NO: 13)
H-Aib-QGTFTSDYSKYLDEKKAK*EFVEWLLEGGPSSG-NH₂;
vi.)
(SEQ ID NO: 14)
H-Aib-QGTFTSDYSK-(nor-V)-LDEKKAK*EFVEWLLEGGPSSG-
NH₂;
and
vii.)
(SEQ ID NO: 15)
H-Aib-QGTFTSDYSI-(nor-V)-LDEKKAK*EFVEWLLEGGPSSG-
NH₂,

wherein the side chain amino (ε amino) group of K* is acylated with a moiety of the formula selected from:

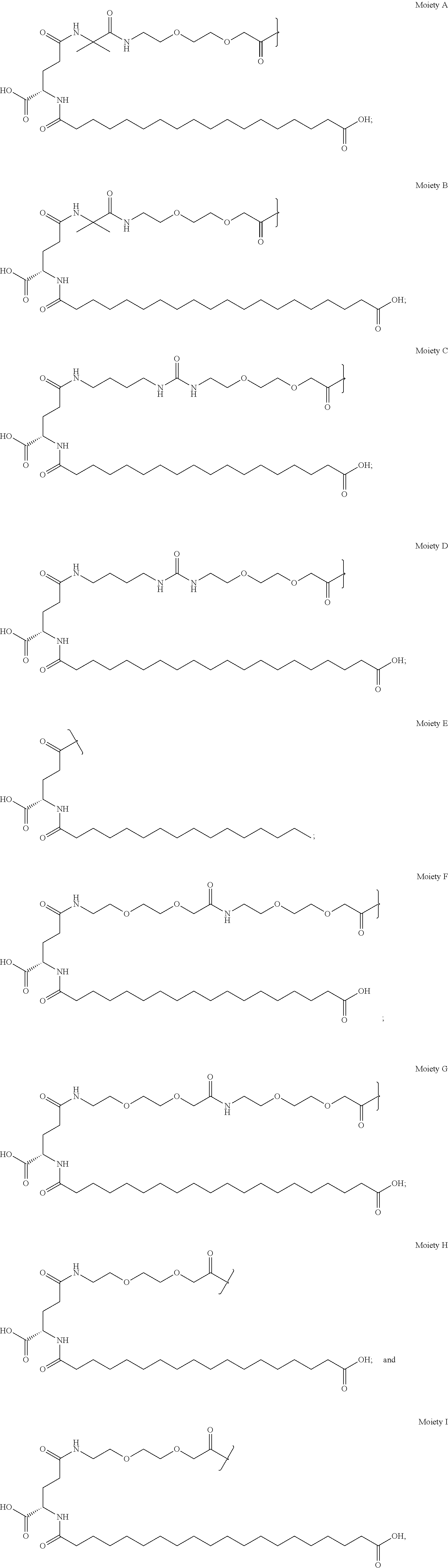

wherein the polypeptide is not selected from SEQ ID NO: 6, SEQ ID NO: 7, SEQ ID NO: 8, or SEQ ID NO: 27.

The sequences of the polypeptides as described herein are represented by either the single-letter code or the three-letter code of the amino acids as approved by the International Union of Pure and Applied Chemistry (IUPAC).

Unless stated otherwise, the present disclosure intends to cover both L and D isomers of the amino acids in the sequences as described herein. However, in certain preferred embodiments, all the amino acids are in the “L” configuration unless indicated otherwise.

In another aspect, the present disclosure provides a polypeptide or pharmaceutically acceptable salt thereof selected from one of the representative compounds in Table 1.

TABLE 1
Representative Polypeptide Compounds

| Com- | | SEQ |
|---|---|---|
| pound | | ID |
| No. | Structure | NO |
| 1 | 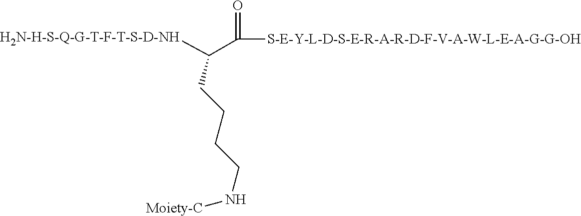 | 16 |
| 2 | 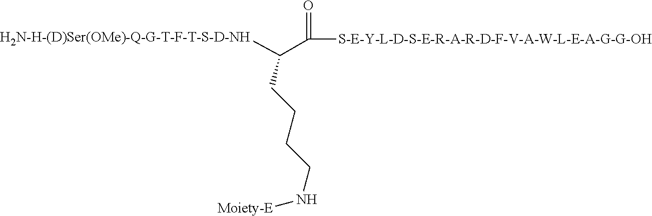 | 17 |
| 3 | 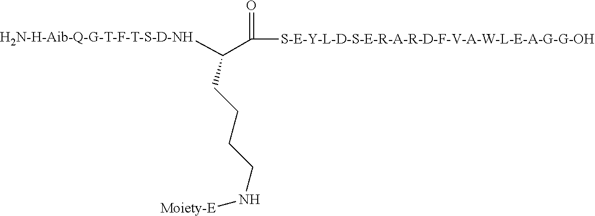 | 18 |
| 4 | 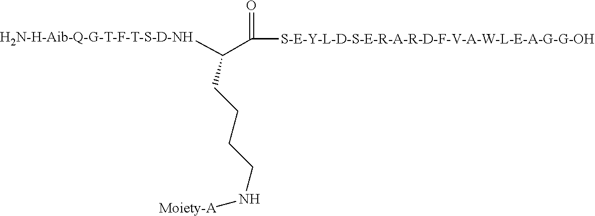 | 19 |
| 5 | 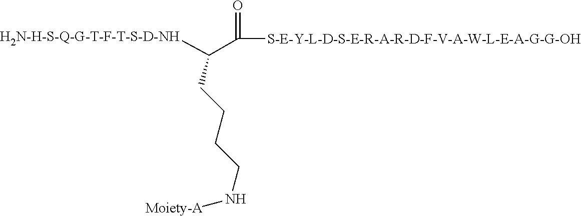 | 20 |
| 6 | 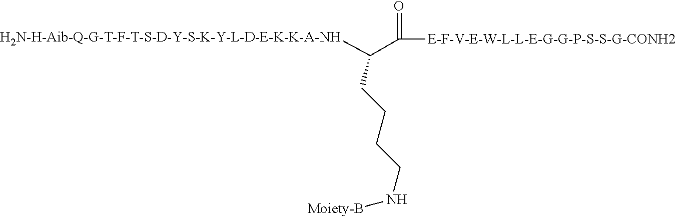 | 21 |
| 7 | 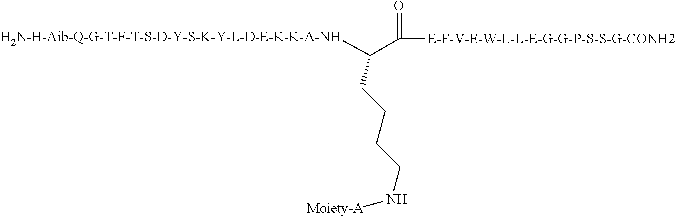 | 22 |
| 8 | 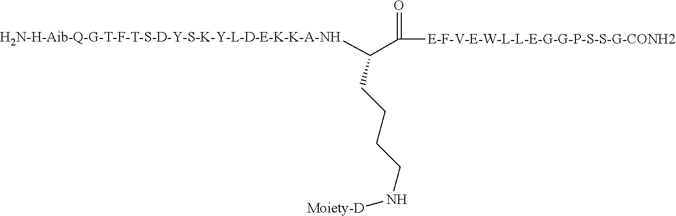 | 23 |
| 9 | 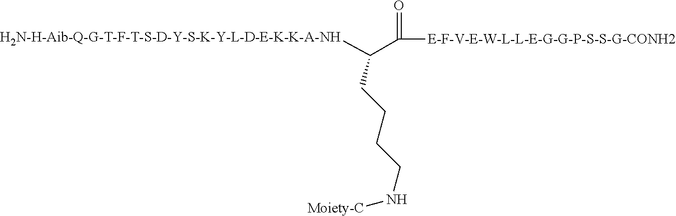 | 24 |
| 10 | 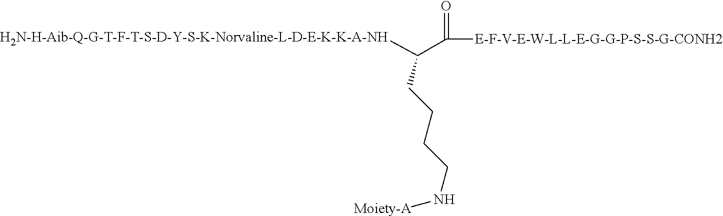 | 25 |
| 11 | 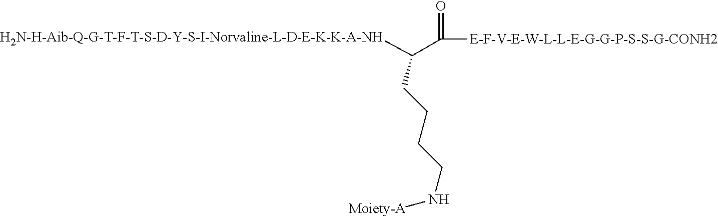 | 26 |
| 12 | 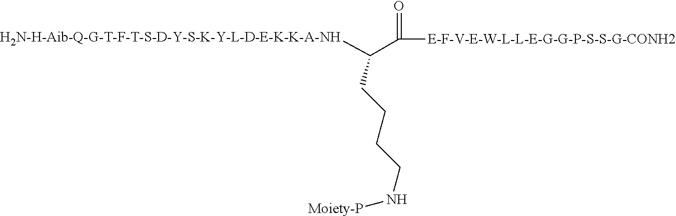 | 27 |

*Unless stated otherwise, all the amino acids mentioned above in Table 1 are in the “L” configuration.

TABLE 2
Structure of Moiety A, Moiety B, Moiety C, Moiety D, Moiety E, Moiety F, Moiety
G, Moiety H, and Moiety I

| | |
|---|---|
| Moiety A | 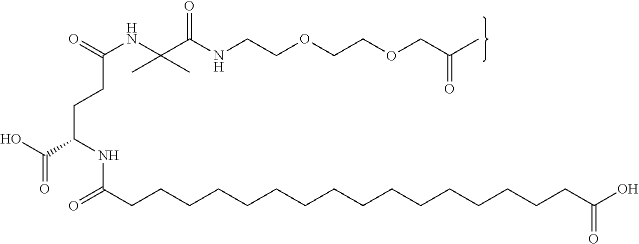 [SMILES: CC(=O)COCCOCCNC(=O)C(C)(C)NC(=O)CC[C@H](NC(=O)CCCCCCCCCCCCCCCCC(=O)O)C(=O)O] |
| Moiety B | 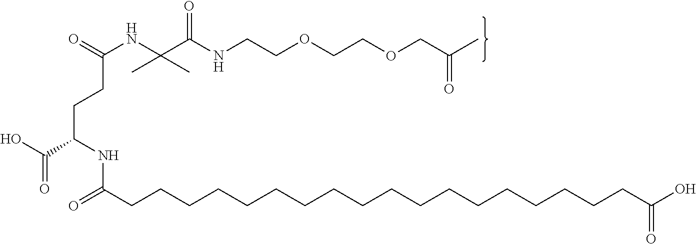 [SMILES: CC(=O)COCCOCCNC(=O)C(C)(C)NC(=O)CC[C@H](NC(=O)CCCCCCCCCCCCCCCCCCC(=O)O)C(=O)O] |
| Moiety C | 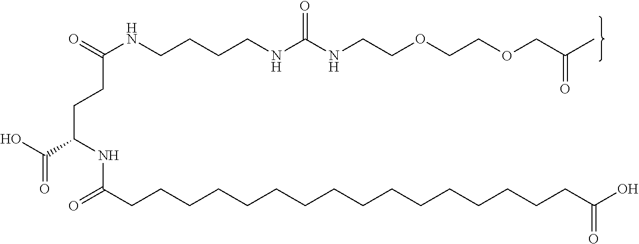 [SMILES: CC(=O)COCCOCCNC(=O)NCCCCNC(=O)CC[C@H](NC(=O)CCCCCCCCCCCCCCCCC(=O)O)C(=O)O] |
| Moiety D | 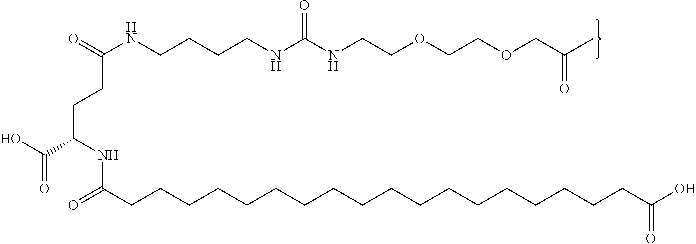 [SMILES: CC(=O)COCCOCCNC(=O)NCCCCNC(=O)CC[C@H](NC(=O)CCCCCCCCCCCCCCCCCCC(=O)O)C(=O)O] |
| Moiety E | 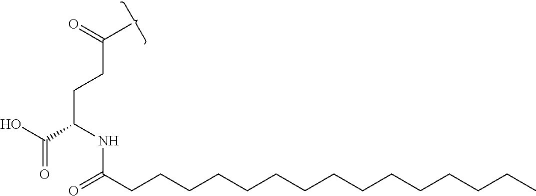 [SMILES: CCCCCCCCCCCCCCCC(=O)N[C@@H](CCC(C)=O)C(=O)O] |
| Moiety F | 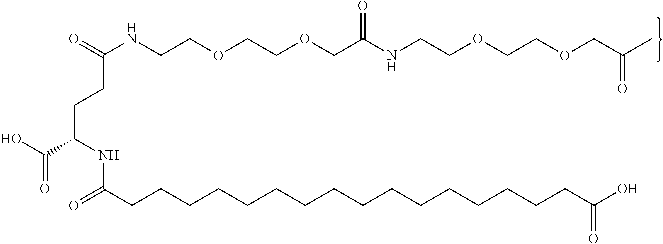 [SMILES: CC(=O)COCCOCCNC(=O)COCCOCCNC(=O)CC[C@H](NC(=O)CCCCCCCCCCCCCCCCC(=O)O)C(=O)O] |
| Moiety G | 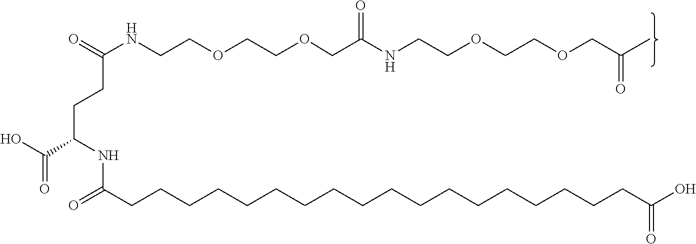 [SMILES: CC(=O)COCCOCCNC(=O)COCCOCCNC(=O)CC[C@H](NC(=O)CCCCCCCCCCCCCCCCCCC(=O)O)C(=O)O] |
| Moiety H | 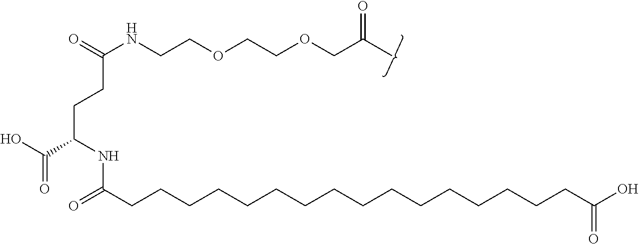 [SMILES: CC(=O)COCCOCCNC(=O)CC[C@H](NC(=O)CCCCCCCCCCCCCCCCC(=O)O)C(=O)O] |
| Moiety I | 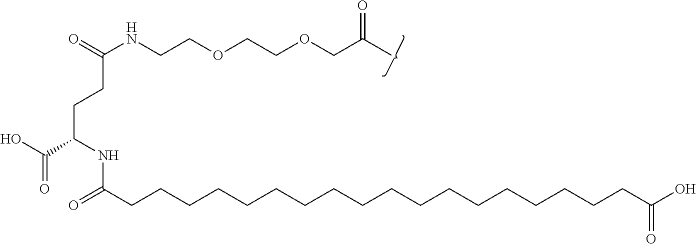 [SMILES: CC(=O)COCCOCCNC(=O)CC[C@H](NC(=O)CCCCCCCCCCCCCCCCCCC(=O)O)C(=O)O] |

In another aspect, the present disclosure provides a polypeptide which is selected from:

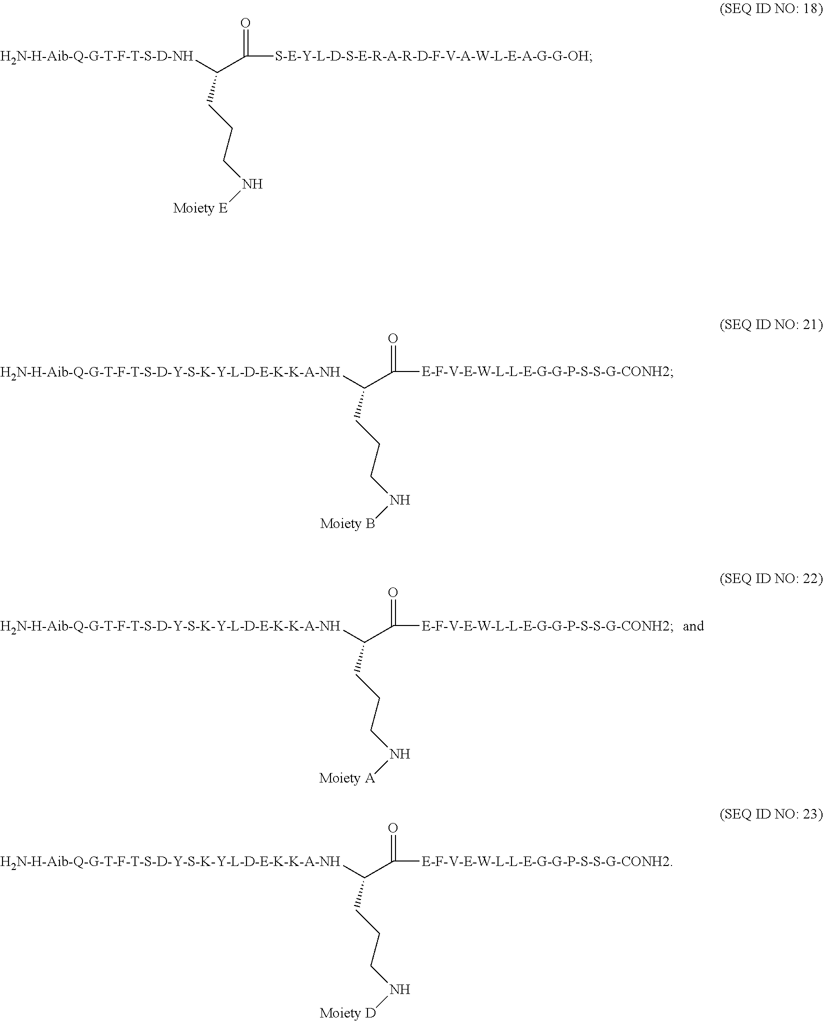

In another aspect, the present disclosure provides pharmaceutical compositions comprising a pharmaceutically acceptable carrier and an incretin analog or a polypeptide as described herein.

In another aspect, the present disclosure provides a method of treating obesity, Type 2 diabetes mellitus (T2DM), metabolic syndrome, metabolic dysfunction-associated steatotic liver disease (MASLD), metabolic dysfunction-associated steatohepatitis (MASH), fibrosis, cardiovascular diseases, and/or hyperlipidemia/dyslipidemia, the method comprising administering to a patient in need of such treatment an incretin analog or a polypeptide as described herein.

In another aspect, the present disclosure provides a method of treating or preventing Type 2 diabetes mellitus (T2DM).

In another aspect, the present disclosure provides a method of treating or preventing hyperlipidemia/dyslipidemia.

In another aspect, the present disclosure provides a method of treating or preventing obesity.

In another aspect, the present disclosure provides a method of treating or preventing metabolic syndromes, non-alcoholic fatty liver diseases (NAFLD), non-alcoholic steatohepatitis (NASH), neurodegenerative disorders, fibrosis, and/or cardiovascular risks.

In one embodiment, the method of treatment comprises administering to a patient in need thereof an effective amount of a polypeptide as described herein or a pharmaceutically acceptable salt thereof.

In another aspect, the present disclosure provides a method of treatment of Type 2 diabetes mellitus (T2DM), the method comprising administering to a patient in need of such treatment an effective amount of a polypeptide as described herein or a pharmaceutically acceptable salt thereof.

In another aspect, the present disclosure provides a method of treatment of obesity, the method comprising administering to a patient in need of such treatment an effective amount of a polypeptide as described herein or a pharmaceutically acceptable salt thereof.

In another aspect, the present disclosure provides a method of treatment of hyperlipidemia/dyslipidemia, the method comprising administering to a patient in need of such treatment an effective amount of a polypeptide as described herein or a pharmaceutically acceptable salt thereof.

In another aspect, the present disclosure provides a pharmaceutical composition comprising a polypeptide as described herein or a pharmaceutically acceptable salt thereof with one or more of a pharmaceutically acceptable carrier, diluent, or excipient.

The compounds of the invention are preferably formulated as pharmaceutical compositions administered by parenteral routes (e.g., subcutaneous, intravenous, intraperitoneal, intramuscular, or transdermal). Such pharmaceutical compositions and processes for preparing the same are well known in the art. See, e.g., “Remington: The Science and 50 Practice of Pharmacy, “edited by D. B. Troy, 21st Edition, Lippincott, Williams & Wilkins, 2006.

In another aspect, the present disclosure provides the polypeptides as described herein or the pharmaceutically acceptable salts thereof for use as a medicament.

In another aspect, the present disclosure provides the polypeptides as described herein or the pharmaceutically acceptable salts thereof for use in the treatment or prevention of Type 2 diabetes mellitus (T2DM).

In another aspect, the present disclosure provides the polypeptides as described herein or the pharmaceutically acceptable salts thereof for use in the treatment or prevention of hyperlipidemia/dyslipidemia.

In another aspect, the present disclosure provides the polypeptides as described herein or the pharmaceutically acceptable salts thereof for use in the treatment or prevention of obesity.

In another aspect, the present disclosure provides the polypeptides as described herein or the pharmaceutically acceptable salts thereof for use in the treatment or prevention of a disease selected from the group consisting of metabolic syndromes, non-alcoholic fatty liver diseases (NAFLD), non-alcoholic steatohepatitis (NASH), neurodegenerative disorders, fibrosis, and cardiovascular risks.

In another aspect, the polypeptide as described herein or the pharmaceutically acceptable salts thereof may be administered simultaneously, separately or sequentially in combination with an effective amount of one or more additional therapeutic agents.

In another aspect, the pharmaceutical composition according to the present disclosure comprises a polypeptide as described herein or a pharmaceutically acceptable salt thereof for use as a medicament.

In another aspect, the pharmaceutical composition according to the present disclosure comprises a polypeptide as described herein or a pharmaceutically acceptable salt thereof for use in the treatment or prevention of Type 2 diabetes mellitus (T2DM).

In another aspect, the pharmaceutical composition according to the present disclosure comprises a polypeptide as described herein or a pharmaceutically acceptable salt thereof for use in the treatment or prevention of hyperlipidemia/dyslipidemia.

In another aspect, the pharmaceutical composition according to the present disclosure comprises a polypeptide as described herein or a pharmaceutically acceptable salt thereof for use in the treatment or prevention of obesity.

In another aspect, the pharmaceutical composition according to the present disclosure comprises a polypeptide as described herein or a pharmaceutically acceptable salt thereof for use in the treatment or prevention of a disease selected from the group consisting of metabolic syndromes, non-alcoholic fatty liver diseases (NAFLD), non-alcoholic steatohepatitis (NASH), neurodegenerative disorders, fibrosis, and cardiovascular risks.

In another aspect, the pharmaceutical composition according to the present disclosure comprises a polypeptide as described herein or a pharmaceutically acceptable salt thereof which is provided simultaneously, separately or sequentially in combination with an effective amount of one or more additional therapeutic agents.

The present disclosure may involve one or more embodiments as described herein. It is to be understood that the embodiments as described herein are illustrative of the present disclosure and are not intended to limit the claims to the specific embodiments exemplified. It is also to be understood that the embodiments as defined herein may be used independently or in conjunction with any definition or any other embodiment as defined herein. Thus, the present disclosure may contemplate all possible combinations and permutations of various independently described embodiments.

Other features of the present disclosure will become apparent to the skilled artisan based on the following examples. Generally speaking, the present disclosure may extend to any novel feature as described herein, including the accompanying claims and drawings. Thus, features, integers, characteristics, compounds, or chemical moieties described in conjunction with a particular aspect, embodiment or example of the present disclosure are to be understood to be applicable to any other aspect, embodiment or example as described herein, unless incompatible therewith.

Moreover, unless stated otherwise, any features as disclosed herein may be replaced by an alternative feature serving the same or a similar purpose.

### Examples

Instruments and analytical methods. Instruments used for characterization and analysis of the compounds as described herein include High Performance Liquid Chromatograph (HPLC) (Waters e2695 Alliance; Detector Waters (2489 UV/Visible)).

Mass instruments: HPLC: Waters e2695 Alliance; and Detector: Acquity-QDa.

The compounds as described herein were purified by preparative HPLC procedures as outlined below.

Preparative HPLC: WATERS 2555 Quaternary gradient module (Max Total Flow: 300 mL/min, Max Pressure: 3000 psi) or Shimadzu LC-8A (Max Total Flow: 150 mL, Max Pressure: 30 Mpa), Column: Phenyl, 10p Flow: 75 mL/min

Mobile Phases:

| | For first | |
|---|---|---|
| | purification | For second purification |
| Mobile Phase A | pH 8.0 phosphate | 1% acetic acid in water |
| | buffer | |
| Mobile Phase B | Acetonitrile | 1% acetic acid in acetonitrile:n- |
| | | propanol (50:50) |
| Gradient | 15 to 45% Mobile | 20 to 50% Mobile Phase B in |
| | Phase B in 300 min | 250 min |

The purity of the compounds as described herein were analyzed by one of the RP-HPLC methods as outlined below.

### HPLC Method A

Column: Xbridge Peptide BEH C18 (4.6 mm×250 mm, 3.5)
Eluent:
   Mobile Phase A: buffer: acetonitrile=900:100
   Mobile Phase B: buffer: acetonitrile=300:700
Buffer: potassium dihydrogen orthophosphate in water and pH is adjusted to 3.0+0.1 with orthophosphoric acid
Flow rate: 0.8 mL/min
Detection: UV detection at 210 nm
Column Temperature: 65 °C
Sample Tray Temperature: 5 °C
Run Time: 90 min

| Time | Mobile Phase A % | Mobile Phase B % |
|---|---|---|
| 0 | 55 | 45 |
| 3 | 55 | 45 |
| 5 | 40 | 60 |
| 60 | 39 | 61 |
| 65 | 0 | 100 |
| 75 | 0 | 100 |
| 75.01 | 55 | 45 |
| 90 | 55 | 45 |

### HPLC Method B

Column: YMC Pack Pro C18 (4.6 mm×250 mm, 3.0)
Eluent:
   Mobile Phase A: buffer: acetonitrile=900:100
   Mobile Phase B: buffer: acetonitrile=300:700
   Buffer: potassium dihydrogen orthophosphate in water and pH is adjusted to 3.0+0.1 with orthophosphoric acid
Flow rate: 1.0 mL/min
Detection: UV detection at 210 nm
Column Temperature: 50 °C
Sample Tray Temperature: 5 °C
Run Time: 38 min

| Time | Mobile Phase A % | Mobile Phase B % |
|---|---|---|
| 0 | 100 | 0 |
| 5 | 100 | 0 |
| 30 | 0 | 100 |
| 32 | 0 | 100 |
| 32.1 | 100 | 0 |
| 38 | 100 | 0 |

### HPLC Method C

Column: X-Select CSH C18, 130A°, 2.5 pm, (4.6×150)mm
Eluent:
   Mobile Phase A: buffer: acetonitrile=900:100
   Mobile Phase B: buffer: acetonitrile=300:700
   Buffer: potassium dihydrogen orthophosphate in water; a trimethylamine is added; and pH is adjusted to 2.5±0.1 with orthophosphoric acid
Flow rate: 0.5 mL/min
Detection: UV detection at 214 nm
Column Temperature: 60 °C
Sample Tray Temperature: 5 °C
Run Time: 90 min

| Time | Mobile Phase A % | Mobile Phase B % |
|---|---|---|
| 0 | 55 | 45 |
| 6 | 55 | 45 |
| 10 | 40 | 60 |
| 80 | 39 | 61 |
| 80.1 | 0 | 100 |
| 85 | 0 | 100 |
| 85.1 | 55 | 45 |
| 90 | 55 | 45 |

### HPLC Method D

Column: X-Select CSH C18, 130A°, 2.5 pm, (4.6×150)mm
Eluent:
   Mobile Phase A: buffer: acetonitrile=900:100
   Mobile Phase B: buffer: acetonitrile=300:700
   Buffer: potassium dihydrogen orthophosphate in water; trimethylamine is added; and pH is adjusted to 2.5±0.1 with orthophosphoric acid
Flow rate: 0.8 mL/min
Detection: UV detection at 210 nm
Column Temperature: 60 °C
Sample Tray Temperature: 5 °C
Run Time: 33 min

| Time | Mobile Phase A % | Mobile Phase B % |
|---|---|---|
| 0 | 60 | 40 |
| 5 | 55 | 45 |
| 25 | 20 | 80 |
| 25.1 | 60 | 40 |
| 33.0 | 60 | 40 |

### Method of Preparation

### Example A: Preparation of Moiety A-Di-Tert-Butyl Ester

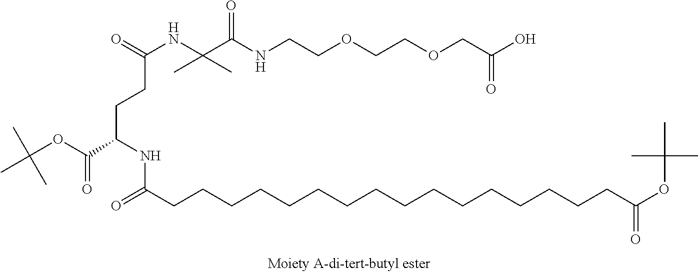 [SMILES: CC(C)(C)OC(=O)CCCCCCCCCCCCCCCCC(=O)N[C@@H](CCC(=O)NC(C)(C)C(=O)NCCOCCOCC(=O)O)C(=O)OC(C)(C)C]

Moiety A-di-tert-butyl ester was prepared using solid phase synthesis. 2-[2-(2-Fmoc-aminoethoxy)ethoxy]acetic acid was attached to 2-chlorotrityl chloride resin in the presence of DIPEA to yield 2-[2-(2-Fmoc-aminoethoxy)ethoxy]acetic acid-2-chlorotrityl-resin. The Fmoc protecting group was removed by selective de-blocking of amino group using piperidine followed by coupling with Fmoc-Aib-OH in THF using DIPC and HOBt which yielded 2-[2-[2-[(2-Fmoc-amino-2-methyl-propanoyl)amino]ethoxy]ethoxy]acetic acid-2-chlorotrityl-resin. The Fmoc group was removed by selective de-blocking using piperidine and the free amino group was coupled with Fmoc-Glu-OtBu using HOBt and DIPC to yield 2-[2-[2-[[2-[[(4S)-4-Fmoc-amino-5-tert-butoxy-5-oxo-pentanoyl]amino]-2-methyl-propanoyl]amino]ethoxy]ethoxy]acetic acid-2-chlorotrityl-resin. The Fmoc group ofthe resultant compound was selectively de-blocked using piperidine, and the free amino group was then coupled with octadecanedioic acid mono tert butyl ester to give 2-[2-[2-[[2-[[(4S)-5-tert-butoxy-4-[(18-tert-butoxy-18-oxo-octadecanoyl)amino]-5-oxo-pentanoyl]amino]-2-methyl-propanoyl]- amino]ethoxy]ethoxy]acetic acid-2-chlorotrityl-resin. The intermediate was then cleaved from 2-chlorotrityl-resin using trifluoroethanol:DCM (1:1) to obtain 2-[2-[2-[[2-[[(4S)-5-tert-butoxy-4-[(18-tert-butoxy-18-oxo-octadecanoyl)amino]-5-oxo-pentanoyl]amino]-2-methyl-propanoyl]amino]ethoxy]ethoxy]acetic acid (Moiety A-di-tert-butyl ester). LCMS=m/z: 786.39 (M+H+).

### Preparation of Moiety A-OSu

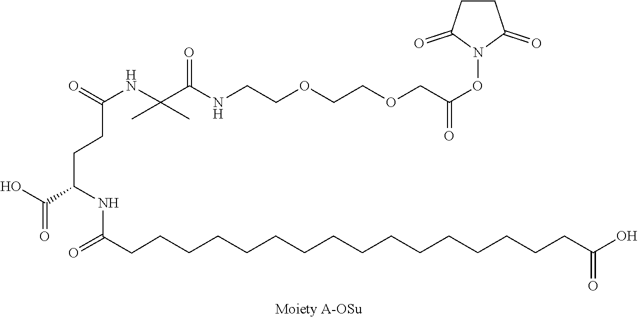 [SMILES: CC(C)(NC(=O)CC[C@H](NC(=O)CCCCCCCCCCCCCCCCC(=O)O)C(=O)O)C(=O)NCCOCCOCC(=O)ON1C(=O)CCC1=O]

The resultant Moiety A-di-tert-butyl ester was then reacted with HOSu in the presence of dicyclohexyl carbodiimide (DCC) to yield succinimide protected intermediate, which was de-protected with trifluoroacetic acid to yield the title compound Moiety A-OSu.

### Example B: Preparation of Moiety B-Di-Tert-Butyl Ester

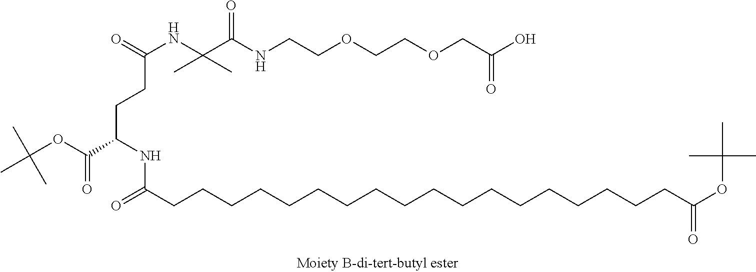 [SMILES: CC(C)(C)OC(=O)CCCCCCCCCCCCCCCCCCC(=O)N[C@@H](CCC(=O)NC(C)(C)C(=O)NCCOCCOCC(=O)O)C(=O)OC(C)(C)C]

Moiety B-di-tert-butyl ester was prepared using the analogous process given in Example A, wherein 20-(tert-butoxy)-20-oxoicosanoic acid was used instead of octadecanedioic acid mono tert butyl ester to give 2-[2-[2-[[2-[[(4S)-5-tert-butoxy-4-[(20-tert-butoxy-20-oxo-icosanoyl)amino]-5-oxo-pentanoyl]amino]-2-methyl-propanoyl]amino]ethoxy]ethoxy]acetic acid-2-chlorotrityl-resin. This intermediate was then cleaved from 2-chlorotrityl-resin using trifluoroethanol:DCM (1:1) to obtain 2-[2-[2-[[2-[[(4S)-5-tert-butoxy-4-[(20-tert-butoxy-20-oxo-icosanoyl)amino]-5-oxo-pentanoyl]amino]-2-methyl-propanoyl]amino]ethoxy]ethoxy]acetic acid (Moiety B-di-tert-butyl ester). LCMS=m/z: 814.10 (M+H+).

### Preparation of Moiety B-OSu

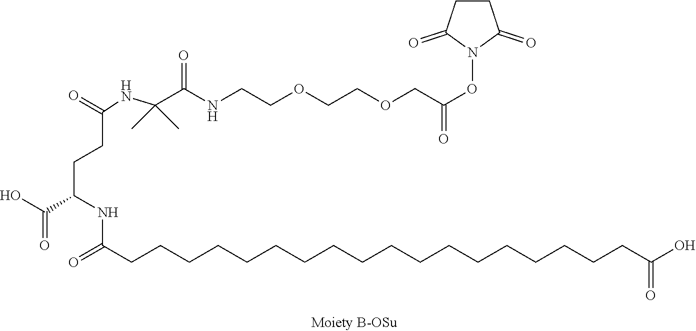 [SMILES: CC(C)(NC(=O)CC[C@H](NC(=O)CCCCCCCCCCCCCCCCCCC(=O)O)C(=O)O)C(=O)NCCOCCOCC(=O)ON1C(=O)CCC1=O]

The resultant Moiety B-di-tert-butyl ester was then reacted with HOSu in the presence of dicyclohexyl carbodiimide (DCC) to yield succinimide protected intermediate, which was de-protected with trifluoroacetic acid to yield the title compound Moiety B-OSu.

### Example C: Preparation of Moiety C-Di-Tert-Butyl Ester

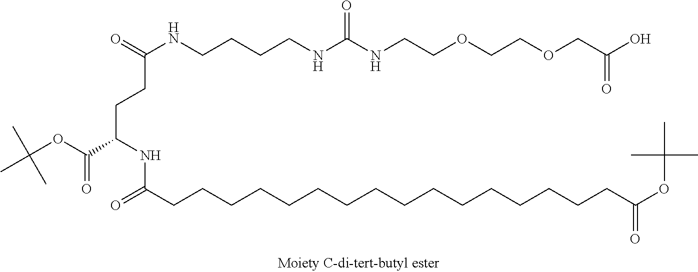 [SMILES: CC(C)(C)OC(=O)CCCCCCCCCCCCCCCCC(=O)N[C@@H](CCC(=O)NCCCCNC(=O)NCCOCCOCC(=O)O)C(=O)OC(C)(C)C]

Moiety C-di-tert-butyl ester was prepared using solid phase synthesis. 2-[2-(2-Fmoc-aminoethoxy)ethoxy]acetic acid was attached to 2-chlorotrityl chloride resin in the presence of DIPEA to yield 2-[2-(2-Fmoc-aminoethoxy)ethoxy]acetic acid-2-chlorotrityl-resin. The Fmoc protecting group was removed by selective de-blocking of amino group using piperidine, and the free amino group was then activated using p-nitrophenylchlroformate in THF and DIPEA followed by reaction with Fmoc-amino butylamine hydrochloride salt in THF:DMAc and DIPEA, which yielded 2-[2-[2-(4-Fmoc-aminobutylcarbamoylamino)ethoxy]ethoxy]acetic acid-2-chlorotrityl-resin. The Fmoc group was removed by selective de-blocking using piperidine, and the free amino group was then coupled to Fmoc-Glu-OtBu using of HOBt and DIPC, which yielded 2-[2-[2-[4-[[(4S)-4-Fmoc-amino-5-tert-butoxy-5-oxo-pentanoyl]amino]butylcarbamoylamino]ethoxy]ethoxy]acetic acid-2-chlorotrityl-resin. The resultant 2-[2-[2-[4-[[(4S)-4-Fmoc-amino-5-tert-butoxy-5-oxo-pentanoyl]amino]-butylcarbamoylamino]ethoxy]ethoxy]acetic acid-2-chlorotrityl-resin was selectively deblocked using piperidine, and then coupled with octadecanedioic acid mono tert-butyl ester to give intermediate 2-[2-[2-[4-[[(4S)-5-tert-butoxy-4-[(18-tert-butoxy-18-oxo-octadecanoyl)amino]-5-oxo-pentanoyl]amino]butylcarbamoylamino]ethoxy]ethoxy]acetic acid-2-chlorotrityl-resin. The intermediate was then cleaved from 2-chlorotrityl-resin using trifluoroethanol:DCM (1:1) to obtain 2-[2-[2-[4-[[(4S)-5-tert-butoxy-4-[(18-tert-butoxy-18-oxo-octadecanoyl)amino]-5-oxo-pentanoyl]amino]butylcarbamoylamino]ethoxy]ethoxy]acetic acid (Moiety C-di-tert-butyl ester). LCMS=m/z: 814.56 (M+H+).

### Preparation of Moiety C-OSu

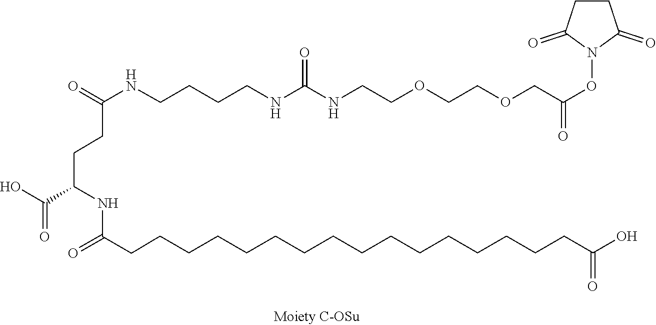 [SMILES: O=C(O)CCCCCCCCCCCCCCCCC(=O)N[C@@H](CCC(=O)NCCCCNC(=O)NCCOCCOCC(=O)ON1C(=O)CCC1=O)C(=O)O]

The resultant Moiety C-di-tert-butyl ester was then reacted with HOSu in the presence of dicyclohexyl carbodiimide (DCC) to yield succinimide protected intermediate, which was de-protected with trifluoroacetic acid to yield the title compound Moiety C-OSu.

### Example D: Preparation of Moiety D-Di-Tert-Butyl Ester

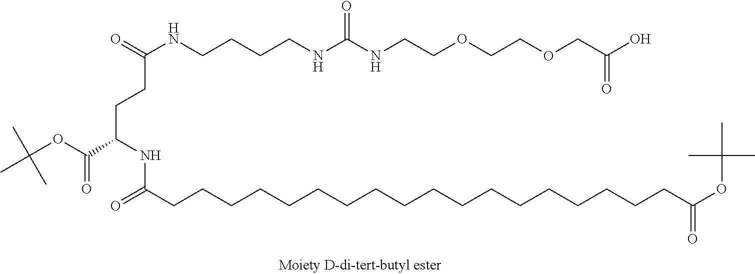 [SMILES: CC(C)(C)OC(=O)CCCCCCCCCCCCCCCCCCC(=O)N[C@@H](CCC(=O)NCCCCNC(=O)NCCOCCOCC(=O)O)C(=O)OC(C)(C)C]

Moiety B-di-tert-butyl ester was prepared using the analogous process given in Example C, wherein 20-(tert-butoxy)-20-oxoicosanoic acid was used instead of octadecanedioic acid mono tert butyl ester to give intermediate 2-[2-[2-[4-[[5-tert-butoxy-4-[(20-tert-butoxy-20-oxo-icosanoyl)amino]-5-oxo-pentanoyl]amino]butylcarbamoylamino]ethoxy]ethoxy]acetic acid-2-chlorotrityl-resin. The intermediate was then cleaved from 2-chlorotrityl-resin using trifluoroethanol:DCM (1:1) to obtain 2-[2-[2-[4-[[5-tert-butoxy-4-[(20-tert-butoxy-20-oxo-icosanoyl)amino]-5-oxo-pentanoyl]amino]butylcarbamoylamino]ethoxy]ethoxy]acetic acid (Moiety D-di-tert-butyl ester). LCMS=m/z: 843.14 (M+H⁺).

### Preparation of Moiety D-OSu

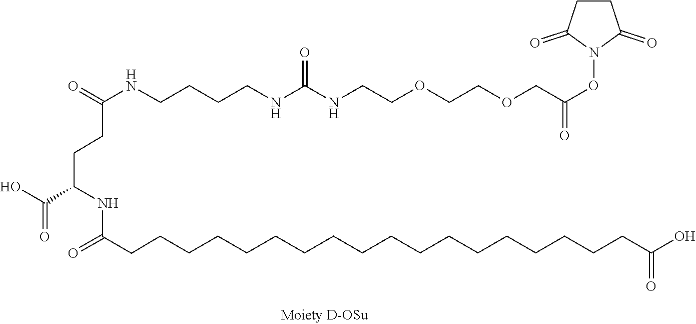 [SMILES: O=C(O)CCCCCCCCCCCCCCCCCCC(=O)N[C@@H](CCC(=O)NCCCCNC(=O)NCCOCCOCC(=O)ON1C(=O)CCC1=O)C(=O)O]

The resultant Moiety D-di-tert-butyl ester was then reacted with HOSu in the presence of dicyclohexyl carbodiimide (DCC) to yield succinimide protected intermediate, which was de-protected with trifluoroacetic acid to yield the title compound Moiety D-OSu.

### Example E: Preparation of Moiety E-OSu

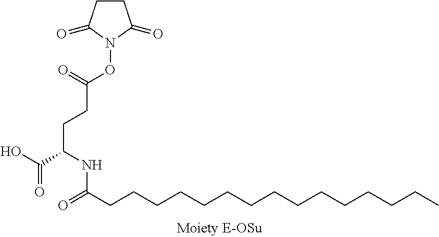 [SMILES: CCCCCCCCCCCCCCCC(=O)N[C@@H](CCC(=O)ON1C(=O)CCC1=O)C(=O)O]

L-Glutamic acid alpha-tert-butyl ester (H-Glu-OtBu) was reacted with palmitic acid in the presence of IBCF and NMM to yield CH₃—(CH₂)₁₄—C(O)-Glu-OtBu, which was then reacted with HOSu in the presence of IBCF and NMM to yield CH₃—(CH₂)₁₄—C(O)-Glu(OSu)-OtBu, which was then de-protected with trifluoroacetic acid to yield Moiety E-OSu.

### Example F: Preparation of Moiety F-Di-Tert-Butyl Ester

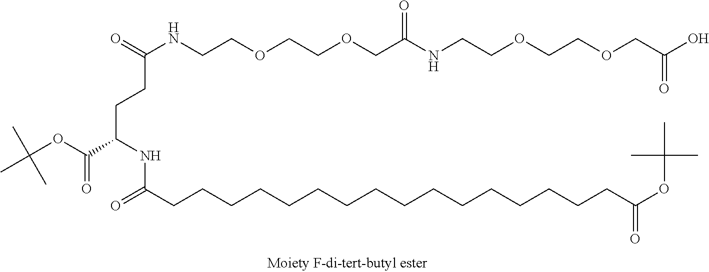 [SMILES: CC(C)(C)OC(=O)CCCCCCCCCCCCCCCCC(=O)N[C@@H](CCC(=O)NCCOCCOCC(=O)NCCOCCOCC(=O)O)C(=O)OC(C)(C)C]

Moiety F-di-tert-butyl ester was prepared using solid phase synthesis. 2-[2-(2-Fmoc-aminoethoxy)ethoxy]acetic acid was attached to 2-chlorotrityl chloride resin in the presence of DIPEA to yield 2-[2-(2-Fmoc-aminoethoxy)ethoxy]acetic acid-2-chlorotrityl-resin. The Fmoc protecting group was removed by selective de-blocking of amino group using piperidine followed by coupling with 2-[2-(2-Fmoc-aminoethoxy)ethoxy]acetic acid in THF using DIPC and HOBt, which yielded {(Fmoc-amino-ethoxy)-ethoxy}-acetyl-{(-amino-ethoxy)-ethoxy}-acetic acid-2-chlorotrityl-resin. The Fmoc group was removed by selective de-blocking using piperidine, and the free amino group was coupled with Fmoc-Glu-OtBu using HOBt and DIPC to yield Fmoc-Glu({(amino-ethoxy)-ethoxy}-acetyl-{(−amino-ethoxy)-ethoxy}-acetic acid-2-chlorotrityl-resin)-OtBu. The Fmoc group of the resultant compound was selectively de-blocked using piperidine and the free amino group was then coupled with octadecanedioic acid mono tert butyl ester to give 2-[2-[2-[[2-[2-[2-[[5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid-2-chlorotrityl-resin. The intermediate was then cleaved from 2-chlorotrityl-resin using trifluoroethanol:DCM (1:1) to obtain 2-[2-[2-[2-[2-[2-[[5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid (Moiety F-di-tert-butyl ester). LCMS=m/z: 846.10 (M+H⁺).

### Preparation of Moiety F-OSu

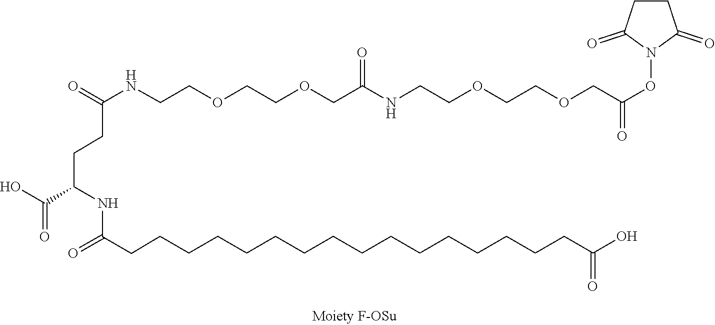 [SMILES: O=C(O)CCCCCCCCCCCCCCCCC(=O)N[C@@H](CCC(=O)NCCOCCOCC(=O)NCCOCCOCC(=O)ON1C(=O)CCC1=O)C(=O)O]

The resultant Moiety F-di-tert-butyl ester was then reacted with HOSu in the presence of dicyclohexyl carbodiimide (DCC) to yield succinimide protected intermediate, which was de-protected with trifluoroacetic acid to yield the title compound Moiety F-OSu.

### Example G: Preparation Moiety G

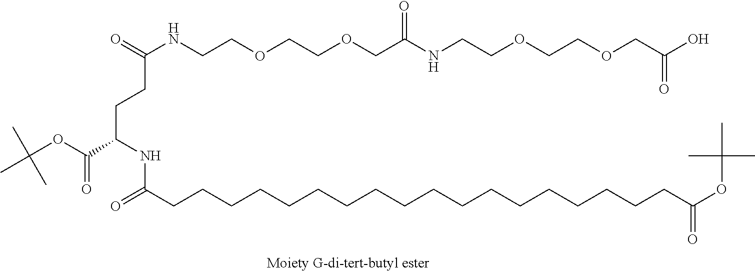 [SMILES: CC(C)(C)OC(=O)CCCCCCCCCCCCCCCCCCC(=O)N[C@@H](CCC(=O)NCCOCCOCC(=O)NCCOCCOCC(=O)O)C(=O)OC(C)(C)C]

Moiety G-di-tert-butyl ester was prepared using the analogous process given in Example F, wherein 20-(tert-butoxy)-20-oxoicosanoic acid was used instead of octadecanedioic acid mono tert butyl ester to give intermediate 2-[2-[2-[[2-[2-[2-[[5-tert-butoxy-4-[(20-tert-butoxy-20-oxo-icosanoyl)amino]-5-oxo-pentanoyl]amino]ethoxy]ethoxy]acetyl]amino]ethoxy]ethoxy]acetic acid-2-chlorotrityl-resin. The intermediate was then cleaved from 2-chlorotrityl-resin using trifluoroethanol:DCM (1:1) to obtain 2-[2-[2-[[2-[2-[2-[[5-tert-butoxy-4-[(20-tert-butoxy-20-oxo-icosanoyl)amino]-5-oxo-pentanoyl]amino]ethoxy]ethoxy]acetyl]amino]ethoxy]ethoxy]acetic acid (Moiety G-di-tert-butyl ester). LCMS=m/z: 874.15 (M+H⁺).

### Preparation of Moiety G-OSu

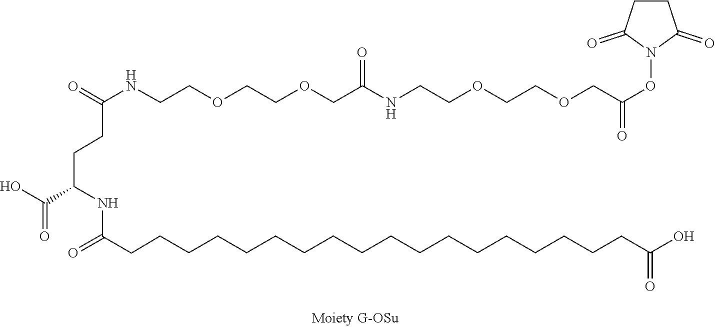 [SMILES: O=C(O)CCCCCCCCCCCCCCCCCCC(=O)N[C@@H](CCC(=O)NCCOCCOCC(=O)NCCOCCOCC(=O)ON1C(=O)CCC1=O)C(=O)O]

The resultant Moiety G-di-tert-butyl ester was then reacted with HOSu in the presence of dicyclohexyl carbodiimide (DCC) to yield succinimide protected intermediate, which was de-protected with trifluoroacetic acid to yield the title compound Moiety G-OSu.

### Example H: Preparation Moiety H-Di-Tert-Butyl Ester

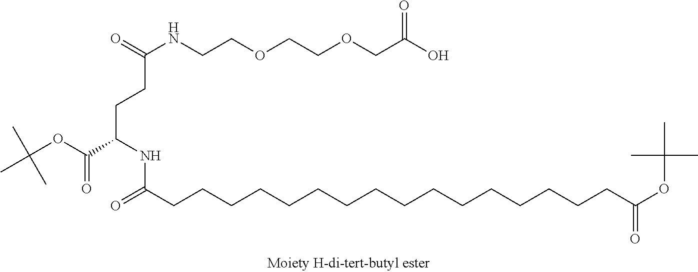 [SMILES: CC(C)(C)OC(=O)CCCCCCCCCCCCCCCCC(=O)N[C@@H](CCC(=O)NCCOCCOCC(=O)O)C(=O)OC(C)(C)C]

Moiety H-di-tert-butyl ester was prepared using solid phase synthesis using 2-chlorotrityl chloride resin. 2-[2-(2-Fmoc-aminoethoxy)ethoxy]acetic acid was attached to 2-chlorotrityl chloride resin in the presence of DIPEA to yield 2-[2-(2-Fmoc-aminoethoxy)ethoxy]acetic acid-2-chlorotrityl-resin. The Fmoc protecting group was removed by selective de-blocking of amino group using piperidine followed by coupling with Fmoc-Glu-OtBu using HOBt and DIPC to yield 2-[2-[2-[[(4S)-5-tert-butoxy-4-(9H-fluoren-9-ylmethoxycarbonylamino)-5-oxo-pentanoyl]amino]ethoxy]ethoxy]acetic acid-2-chlorotrityl-resin. The Fmoc group of the resultant compound was selectively de-blocked using piperidine and the free amino group was then coupled with octadecanedioic acid mono tert butyl ester to give 2-[2-[2-[[(4S)-5-tert-butoxy-4-[(18-tert-butoxy-18-oxo-octadecanoyl)amino]-5-oxo-pentanoyl]amino]ethoxy]ethoxy]acetic acid 2-chlorotrityl-resin. The intermediate was then cleaved from 2-chlorotrityl-resin using trifluoroethanol:DCM (1:1) to obtain 2-[2-[2-[[(4S)-5-tert-butoxy-4-[(18-tert-butoxy-18-oxo-octadecanoyl)amino]-5-oxo-pentanoyl]amino]ethoxy]ethoxy]acetic acid (Moiety H-di-tert-butyl ester). LCMS=m/z: 700.94 (M+H⁺).

### Preparation of Moiety H-OSu

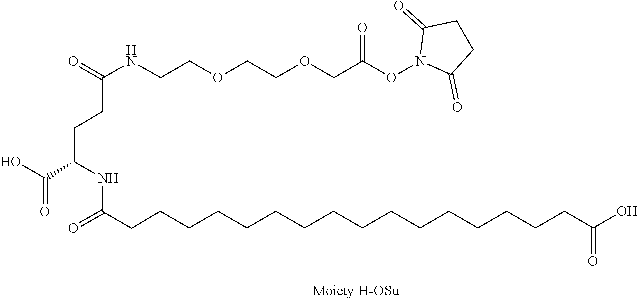 [SMILES: O=C(O)CCCCCCCCCCCCCCCCC(=O)N[C@@H](CCC(=O)NCCOCCOCC(=O)ON1C(=O)CCC1=O)C(=O)O]

The resultant Moiety H-di-tert-butyl ester was then reacted with HOSu in the presence of dicyclohexyl carbodiimide (DCC) to yield succinimide protected intermediate, which was de-protected with trifluoroacetic acid to yield the title compound Moiety H-OSu.

### Example I: Preparation Moiety I

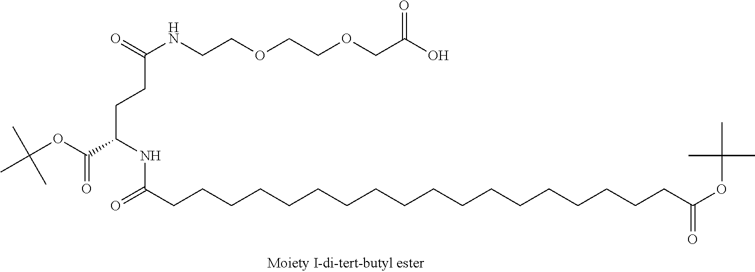 [SMILES: CC(C)(C)OC(=O)CCCCCCCCCCCCCCCCCCC(=O)N[C@@H](CCC(=O)NCCOCCOCC(=O)O)C(=O)OC(C)(C)C]

Moiety 1-di-tert-butyl ester was prepared using the analogous process given in Example H, wherein 20-(tert-butoxy)-20-oxoicosanoic acid was used instead of octadecanedioic acid mono tert butyl ester to give intermediate 2-[2-[2-[[(4S)-5-tert-butoxy-4-[(20-tert-butoxy-20-oxo-icosanoyl)amino]-5-oxo-pentanoyl]amino]ethoxy]ethoxy]acetic acid 2-chlorotrityl-resin. The intermediate was then cleaved from 2-chlorotrityl-resin using trifluoroethanol:DCM (1:1) to obtain 2-[2-[2-[[(4S)-5-tert-butoxy-4-[(20-tert-butoxy-20-oxo-icosanoyl)amino]-5-oxo-pentanoyl]amino]ethoxy]ethoxy]acetic acid (Moiety I-di-tert-butyl ester). LCMS=m/z: 728.99 (M+H⁺).

### Preparation of Moiety I-OSu

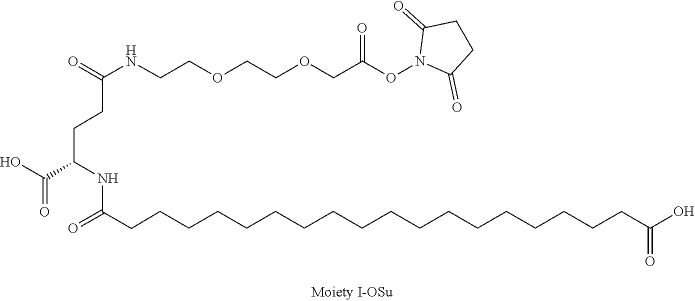 [SMILES: O=C(O)CCCCCCCCCCCCCCCCCCC(=O)N[C@@H](CCC(=O)NCCOCCOCC(=O)ON1C(=O)CCC1=O)C(=O)O]

The resultant Moiety I-di-tert-butyl ester was then reacted with HOSu in the presence of dicyclohexyl carbodiimide (DCC) to yield succinimide protected intermediate, which was de-protected with trifluoroacetic acid to yield the title compound Moiety I-OSu.

### Example 1: Synthesis of Compound 1

### Part A: Synthesis of Linear Peptide Backbone

The peptide backbone was synthesized by solid-phase method. The starting resin used for synthesis was Wang resin. Fmoc protected Gly-OH was used for coupling with the Wang resin. The coupling was performed by using diisopropylcarbodiimide, N-hydroxybenzotriazole (DIC-HOBt) as coupling reagent in the presence of 4-dimethylaminopyridine (DMAP), which yielded Fmoc-Gly-Wang Resin. Acetic anhydride and diisopropylethyl amine/pyridine were used to terminate the uncoupled amino groups at every amino acid coupling. Selective de-blocking of amino group of Fmoc-Gly-Wang Resin using piperidine followed by coupling with Fmoc-Gly-OH using HOBt and DIPC yielded Fmoc-Gly-Gly-Wang Resin. This completes one cycle.

The above 3 steps: selective capping, selective deblocking of Fmoc-protection of amino acid attached to the resin and coupling of next amino acid residue in sequence with Fmoc-protected amino group were repeated for remaining 28 amino acid residues. The selective deblocking (deprotection of Fmoc group) was done using piperidine and coupling with next Fmoc protected amino acid was done using HOBt/DIPC. The side chain of the Fmoc-protected amino acids were protected orthogonally (hydroxyl group of Serine, Tyrosine or Threonine were protected with tert-butyl(-tBu) group; amino and guanido group of Lysine and Arginine were protected with tert-butyloxycarbonyl (-Boc) and 2,2,4,6,7-pentamethyldihydrobenzofuran-5-sulfonyl (-Pbf) group, respectively, the imidazole of histidine was protected with tert-butyloxycarbonyl (-Boc) and carboxylic acid groups of aspartic acid or glutamic acid were protected with (-tBu)group and amide group of glutamine was protected with trityl (-Trt) group). The above mentioned 2 steps: selective deblocking and then coupling with next Fmoc protected amino acid were performed to get Fmoc-His(Boc)-Ser(tBu)-Gln(Trt)-Gly-Thr(tBu)-Phe-Thr(tBu)-Ser(tBu)-Asp(OtBu)-Lys(Boc)-Ser(tBu)-Glu(OtBu)-Tyr(tBu)-Leu-Asp(OtBu)-Ser(tBu)-Glu(OtBu)- Arg(Pbf)-Ala-Arg(Pbf)-Asp(OtBu)-Phe-Val-Ala-Trp-Leu-Glu(OtBu)-Ala-Gly-Gly-resin.

De-blocking of Fmoc-His(Boc)-Ser(tBu)-Gln(Trt)-Gly-Thr(tBu)-Phe-Thr(tBu)-Ser(tBu)-Asp(OtBu)-Lys(Boc)-Ser(tBu)-Glu(OtBu)-Tyr(tBu)-Leu-Asp(OtBu)-Ser(tBu)-Glu(OtBu)-Arg(Pbf)-Ala-Arg(Pbf)-Asp(OtBu)-Phe-Val-Ala-Trp-Leu-Glu(OtBu)- Ala-Gly-Gly-resin using piperidine followed by cleavage and de-protection using trifluoroacetic acid with ethane-1,2-dithiol and triisopropylsilane resulted in crude H-His-Ser-Gln-Gly-Thr-Phe-Thr-Ser-Asp-Lys(ε-NH₂)-Ser-Glu-Tyr-Leu-Asp-Ser-Glu-Arg-Ala-Arg-Asp-Phe-Val-Ala-Trp-Leu-Glu-Ala-Gly-Gly-OH, which was purified by Preparative HPLC.

### Part B: Grafting of Activated Fatty Acid Chain Over Liner Peptide

The activated fatty acid chain Moiety C-Osu was grafted on the purified linear peptide as obtained in Part A in water:acetonitrile at pH about 11 to yield crude title peptide Compound 1, which was purified by preparative HPLC.
Mass (LCMS): m/z=1012.36 (MH4 4+); Calculated Mass=4045.40; and HPLC Purity (Method B): 94.48%.

### Example 2: Synthesis of Compound 2

### Part A: Synthesis of Linear Peptide Backbone

The linear peptide backbone of Compound 2 was prepared by solid phase method as per the analogous process given in Example 1 Part A, wherein Fmoc-D-Ser(OMe)-OH was used at position 2 instead of Fmoc-Ser(tBu)-OH.

### Part B: Grafting of Activated Fatty Acid Chain Over Liner Ppeptide

The activated fatty acid chain Moiety E-OSu was grafted on purified linear peptide: H-His-D-Ser(OMe)-Gln-Gly-Thr-Phe-Thr-Ser-Asp-Lys(ε-NH₂)-Ser-Glu-Tyr-Leu-Asp-Ser-Glu-Arg-Ala-Arg-Asp-Phe-Val-Ala-Trp-Leu-Glu-Ala-Gly-Gly-OH as obtained in Part A, in water:acetonitrile at pH about 11 to yield crude title peptide Compound 2, which was purified by preparative HPLC.

Mass (LCMS): m/z=936.10 (MH4 4+); Calculated Mass=3739.96; and HPLC Purity (Method B): 95.05%.

### Example 3: Synthesis of Compound 3

### Part A: Synthesis of Linear Peptide Backbone

The linear peptide backbone of Compound 3 was prepared by solid phase method as per the analogous process given in Example 1 Part A, wherein Fmoc-Aib-OH was used at position 2 instead of Fmoc-Ser(tBu)-OH.

### Part B: Grafting of Activated Fatty Acid Chain Over Liner Peptide

The activated fatty acid chain Moiety E-OSu was grafted on purified linear peptide: H-His-Aib-Gln-Gly-Thr-Phe-Thr-Ser-Asp-Lys(ε-NH₂)-Ser-Glu-Tyr-Leu-Asp-Ser-Glu-Arg-Ala-Arg-Asp-Phe-Val-Ala-Trp-Leu-Glu-Ala-Gly-Gly-OH as obtained in Part A, in water:acetonitrile at pH about 11 to yield crude title peptide Compound 3, which was purified by preparative HPLC.

Mass (LCMS): m/z=932.13 (MH4 4+); Calculated Mass=3724.48; and HPLC Purity (Method B): 96.1%.

### Example 4: Synthesis of Compound 4

### Part A: Synthesis of Linear Peptide Backbone

The linear peptide backbone of Compound 4 was prepared by solid phase method as per the analogous process given in Example 3 Part A.

### Part B: Grafting of Activated Fatty Acid Chain Over Liner Peptide

The activated fatty acid chain Moiety A-OSu was grafted on purified linear peptide: H-His-Aib-Gln-Gly-Thr-Phe-Thr-Ser-Asp-Lys(ε-NH₂)-Ser-Glu-Tyr-Leu-Asp-Ser-Glu-Arg-Ala-Arg-Asp-Phe-Val-Ala-Trp-Leu-Glu-Ala-Gly-Gly-OH as obtained in Part A, in water:acetonitrile at pH about 11 to yield crude title peptide Compound 4, which was purified by preparative HPLC.
Mass (LCMS): m/z=1004.34 (MH4 4+); Calculated Mass=4013.33; and HPLC Purity (Method D): 96.93%.

### Example 5: Synthesis of Compound 5

### Part A: Synthesis of Linear Peptide Backbone

The linear peptide backbone of Compound 4 was prepared by solid phase method as per the analogous process given in Example 1 Part A.

### Part B: Grafting of Activated Fatty Acid Chain Over Liner Peptide

The activated fatty acid chain Moiety A-OSu was grafted on purified linear peptide: H-His-Ser-Gln-Gly-Thr-Phe-Thr-Ser-Asp-Lys(ε-NH₂)-Ser-Glu-Tyr-Leu-Asp-Ser-Glu-Arg-Ala-Arg-Asp-Phe-Val-Ala-Trp-Leu-Glu-Ala-Gly-Gly-OH as obtained in Part A, in water:acetonitrile at pH about 11 to yield crude title peptide Compound 5, which was purified by preparative HPLC.
Mass (LCMS): m/z=1004.96 (MH4 4+); Calculated Mass=4015.81; and HPLC Purity (Method B): 97.33%.

### Example 6: Synthesis of Compound 6

### Part A: Synthesis of Linear Peptide Backbone

Compound 6 was synthesized by solid-phase method. The starting resin used for synthesis was Fmoc-Rink amide resin. Selectively de-blocking of Fmoc protected amino group of Rink amide resin using piperidine followed by coupling of Fmoc-Gly-OH with the Rink amide resin. The coupling was performed by using diisopropylcarbodiimide, N-hydroxybenzotriazole (DIPC-HOBt) as coupling reagent to yield Fmoc-Gly-Rink amide Resin, which completes the first cycle. Acetic anhydride and diisopropylethyl amine were used to terminate/cap the uncoupled amino groups at every amino acid coupling. Selective de-blocking of amino group of Fmoc-Gly-Rink amide Resin using piperidine, Then coupling with Fmoc-Ser(tBu)-OH using HOBt and DIPC yield Fmoc-Ser(tBu)-Gly-Rink amide Resin, which completes the second cycle.

The above 3 steps: selective capping, deblocking of Fmoc- protection of amino acid attached to the resin and coupling of next amino acid residue in sequence with Fmoc-protected amino group were repeated for remaining 32 amino acid residues. The side chain of the Fmoc-protected amino acids were protected orthogonally (hydroxyl group of Serine, Tyrosine or Threonine were protected with tert-butyl(-tBu) group, amino group of Lysine was protected with tert-butyloxycarbonyl (-Boc) and (4,4-dimethyl-2,6-dioxocyclohex-1-ylidene)-3-methylbutyl (IVDde) group respectively and carboxylic acid groups of aspartic acid or glutamic acid were protected with (-tBu) group and amide group of glutamine was protected with trityl (-Trt) group). The above mentioned three steps: selective capping, deblocking and then coupling with next Fmoc protected amino acid were performed to get Fmoc-His(Boc)-Aib-Gln(Trt)-Gly-Thr(tBu)-Phe-Thr(tBu)-Ser(tBu)-Asp(OtBu)-Tyr(tBu)-Ser(tBu)-Lys(Boc)-Tyr(tBu)-Leu-Asp(OtBu)-Glu(OtBu)-Lys(Boc)-Lys(Boc)-Ala- Lys(IVDde)-Glu(OtBu)-Phe-Val-Glu(OtBu)-Trp-Leu-Leu-Glu(OtBu)-Gly-Gly-Pro-Ser(tBu)-Ser(tBu)-Gly-resin.

De-blocking of Fmoc-His(Boc)-Aib-Gln(Trt)-Gly-Thr(tBu)-Phe-Thr(tBu)-Ser(tBu)-Asp(OtBu)-Tyr(tBu)-Ser(tBu)-Lys(Boc)-Tyr(tBu)-Leu-Asp(OtBu)-Glu(OtBu)-Lys(Boc)-Lys(Boc)-Ala-Lys(IVDde)-Glu(OtBu)-Phe-Val-Glu(OtBu)-Trp-Leu-Leu- Glu(OtBu)-Gly-Gly-Pro-Ser(tBu)-Ser(tBu)-Gly-resin using piperidine. Boc protection of the resulting peptide-resin using Boc anhydride was carried out to yield Boc-His(Boc)-Aib-Gln(Trt)-Gly-Thr(tBu)-Phe-Thr(tBu)-Ser(tBu)-Asp(OtBu)-Tyr(tBu)-Ser(tBu)-Lys(Boc)-Tyr(tBu)-Leu-Asp(OtBu)-Glu(OtBu)-Lys(Boc)-Lys(Boc)-Ala- Lys(IVDde)-Glu(OtBu)-Phe-Val-Glu(OtBu)-Trp-Leu-Leu-Glu(OtBu)-Gly-Gly-Pro-Ser(tBu)-Ser(tBu)-Gly-resin. De-protection of IVDde group of the resulting peptide-resin using hydrazine hydrate yielded the linear peptide-resin: Boc-His(Boc)-Aib-Gln(Trt)-Gly-Thr(tBu)-Phe-Thr(tBu)-Ser(tBu)-Asp(OtBu)-Tyr(tBu)-Ser(tBu)-Lys(Boc)-Tyr(tBu)-Leu-Asp(OtBu)-Glu(OtBu)-Lys(Boc)-Lys(Boc)-Ala-Lys(ε-NH₂)-Glu(OtBu)-Phe-Val-Glu(OtBu)-Trp-Leu-Leu-Glu(OtBu)-Gly-Gly-Pro-Ser(tBu)-Ser(tBu)-Gly-resin.

### Part B: Grafting of Activated Fatty Acid Chain Over Liner Peptide

Moiety B-di-tert-butyl ester was coupled on the linear peptide-resin as obtained in Part A, using diisopropylcarbodiimide, N-hydroxybenzotriazole (DIPC-HOBt) as coupling reagent yielded Compound 6-resin. Cleavage from resin and de-protection using trifluoroacetic acid with ethane-1,2-dithiol, triisopropylsilane followed by purification through preparative HPLC resulted in pure Compound 6.
Mass (LCMS): m/z=1126.36 (MH4 4+); Calculated Mass=4501.41; and HPLC Purity (Method A): 97.16%.

### Example 7: Synthesis of Compound 7

Compound 7 was prepared by solid phase method as per the analogous process given for Example 6, wherein IVDde de-protection was followed by coupling of Moiety A-di-tert-butyl ester, instead of Moiety B-di-tert-butyl ester coupling.
Mass (LCMS): m/z=1120.09 (MH4 4+); Calculated Mass=4476.33; and HPLC Purity (Method A): 98.95%.

### Example 8: Synthesis of Compound 8

Compound 8 was prepared by solid phase method as per the analogous process given for Example 6, wherein IVDde de-protection was followed by coupling of Moiety D-di-tert-butyl ester, instead of Moiety B-di-tert-butyl ester coupling.
Mass (LCMS): m/z=1134.05 (MH4 4+) and Calculated Mass=4532.17.

### Example 9: Synthesis of Compound 9

Compound 9 was prepared by solid phase method as per the analogous process given for Example 6, wherein IVDde de-protection was followed by coupling of Moiety C-di-tert-butyl ester, instead of Moiety B-di-tert-butyl ester coupling.
Mass (LCMS): m/z=1127.22 (MH4 4+) and Calculated Mass=4504.85.

### Example 10: Synthesis of Compound 10

Compound 9 was prepared by solid phase method as per the analogous process given for Example 6, wherein Fmoc-norvaline-OH was used at position 13 instead of Fmoc-Tyr(tBu)-OH and IVDde de-protection was followed by coupling of Moiety A-di-tert-butyl ester, instead of Moiety B-di-tert-butyl ester coupling.
Mass (LCMS): m/z=1103.56 (MH4 4+) and Calculated Mass=4410.21.

### Example 11: Synthesis of Compound 11

Compound 11 was prepared by solid phase method as per the analogous process given for Example 6, wherein (i) Fmoc-Ile-OH was used at position 12 instead of Fmoc-Lys(Boc)-OH, (ii) Fmoc-norvaline-OH was used at position 13 instead of Fmoc-Tyr(tBu)-OH and (iii) IVDde de-protection was followed by coupling of Moiety A-di-tert-butyl ester, instead of Moiety B-di-tert-butyl ester coupling.
Mass (LCMS): m/z=1100.11 (MH4 4+) and Calculated Mass=4396.41.

### Example 25: Synthesis of Compound 25

Compound 25 was prepared by solid phase method as per the analogous process given for Example 6, wherein IVDde de-protection was followed by coupling of Moiety F-di-tert-butyl ester, instead of Moiety B-di-tert-butyl ester coupling.
Mass (LCMS): m/z=1135.11 (MH4 4+); Calculated Mass=4536.41; HPLC Purity (Method A): 98.69%.

### Biological Studies

### Example 1: Oral Glucose Tolerance Test (OGTT) in Rats; Single Injection; 1 Mg/Kg Dose

Study 1: Animals were divided into 4 groups (n=4/group): a normal control group, Cotadutide (1 mg/kg), Compound 1 (1 mg/kg), and Semaglutide (1 mg/kg). Animals were fasted for 12 hours before initiation of OGTT. After 22 hours and 166 hours of subcutaneous injection of test, Cotadutide and Semaglutide, blood glucose was measured with blood glucometer (time 0 measurement). All the animals were given 2 g/kg of glucose solution orally. Blood glucose was measured at 20, 40, 60, 90 and 120 minutes following glucose challenge. Body weight and food consumption were recorded at 48 hr and 154 hr.

Study 2: Animals were divided into 4 groups (n=4/group): a normal control group, Compound 2 (1 mg/kg), Compound 3 (1 mg/kg), and Semaglutide (1 mg/kg). Animals were fasted for 12 hours before initiation of OGTT. After 22 hours and 166 hours of subcutaneous injection of test and Semaglutide, blood glucose was measured with blood glucometer (time 0 measurement). All the animals were given 2 g/kg of glucose solution orally. Blood glucose was measured at 20, 40, 60, 90 and 120 minutes following glucose challenge. Body weight and food consumption were recorded at 48 hr and 154 hr.

TABLE 3
Change in Blood Glucose AUC_{(0-120 min)}
22 hr Post Subcutaneous Injection

| | | | |
|---|---|---|---|
| | | | % Change in Blood |
| | | | Glucose AUC_{(0-120 min)} |
| Treatment | Mean | SD | vs Normal Control |

Study 1 (n = 4)

| | | | |
|---|---|---|---|
| Normal Control | 8293 | 1405 | |
| Cotadutide, 1 mg/kg, s.c | 6100 | 1440 | −26.4 |
| Compound 1, 1 mg/kg, s.c | 6629 | 4190 | −20.1 |
| Semaglutide, 1 mg/kg, s.c | 4666 | 2142 | −43.7 |

Study 2 (n = 4)

| | | | |
|---|---|---|---|
| Normal Control | 9266 | 1827 | |
| Compound 2, 1 mg/kg, s.c | 6089* | 1143 | −34.3 |
| Compound 3, 1 mg/kg, s.c | 3541*** | 1351 | −61.8 |
| Semaglutide, 1 mg/kg, s.c | 3574*** | 936 | −61.4 |

*p < 0.05,
**p < 0.01,
***p < 0.001 vs Normal Control;
one way ANOVA followed by Bonferroni's posttests

TABLE 4
Change in Blood Glucose AUC_{(0-120 min)}
166 hr Post Subcutaneous Injection

| | | | |
|---|---|---|---|
| | | | % Change in Blood |
| | | | Glucose AUC_{(0-120 min)} |
| Treatment | Mean | SD | vs Normal Control |

Study 1 (n = 4)

| | | | |
|---|---|---|---|
| Normal Control | 12244 | 1065 | |
| Cotadutide, 1 mg/kg, s.c | 8479 | 2375 | −30.8 |
| Compound 1, 1 mg/kg, s.c | 8071* | 868 | −34.1 |
| Semaglutide, 1 mg/kg, s.c | 10875 | 2513 | −11.2 |

Study 2 (n = 4)

| | | | |
|---|---|---|---|
| Normal Control | 7743 | 1545 | |
| Compound 2, 1 mg/kg, s.c | 6659 | 588 | −14.0 |
| Compound 3, 1 mg/kg, s.c | 2604**# | 1831 | −66.4 |
| Semaglutide, 1 mg/kg, s.c | 6290 | 1352 | −18.8 |

*p < 0.05,
**p < 0.01,
***p < 0.001 vs Normal Control and #p < 0.05, ##p < 0.01, and ###p < 0.001 vs Semaglutide; One way ANOVA followed by Bonferroni's posttests.

TABLE 5
Effect on Body Weight

| | 48 hr | | 154 hr | |
|---|---|---|---|---|
| Body Weight Reduction (%) | Mean | SD | Mean | SD |
| Study 1 (n = 4) | | | | |
| Normal Control | −0.9 | 2.1 | 3.8 | 2.6 |
| Cotadutide, 1 mg/kg, s.c | −6.5*** | 1.1 | 1.7 | 1.7 |
| Compound 1, 1 mg/kg, s.c | −0.8 | 0.9 | 5.3 | 1.7 |
| Semaglutide, 1 mg/kg s.c | −8.4** | 0.5 | 4.5 | 3.1 |

Study 2 (n = 4)

| | | | | |
|---|---|---|---|---|
| Normal Control | 1.0 | 0.5 | 6.2 | 1.5 |
| Compound 2, 1 mg/kg, s.c | −5.7*** | 2.5 | 2.4* | 1.8 |
| Compound 3, 1 mg/kg, s.c | −6.2***# | 1.8 | −1.6*** | 0.2 |
| Semaglutide, 1 mg/kg s.c | −2.3 | 1.1 | 1.1** | 2.0 |

*p < 0.05,
**p < 0.01,
***p < 0.001 vs Normal Control and #p < 0.05, ##p < 0.01, and ###p < 0.001 vs Semaglutide; One way ANOVA followed by Bonferroni's posttests.

TABLE 6
Effect on Food Consumption

| | 48 hr | | 154 hr | |
|---|---|---|---|---|
| Food Consumption (g) | Mean | SD | Mean | SD |
| Study 1 (n = 4) | | | | |
| Normal Control | 52.9 | 18.5 | 132.2 | 11.8 |
| Cotadutide, 1 mg/kg, s.c | 25.2*** | 5.1 | 127.9** | 8.0 |
| Compound 1, 1 mg/kg, s.c | 54.8 | 4.7 | 170.6 | 15.7 |
| Semaglutide, 1 mg/kg s.c | 19.3** | 3.6 | 147.4** | 15.5 |

Study 2 (n = 4)

| | | | | |
|---|---|---|---|---|
| Normal Control | 29.6 | 3.2 | 87.7 | 7.0 |
| Compound 2, 1 mg/kg, s.c | 13.8*** | 3.1 | 60.5** | 8.7 |
| Compound 3, 1 mg/kg, s.c | 10.7***# | 3.8 | 42.0***# | 7.1 |
| Semaglutide, 1 mg/kg s.c | 19.8** | 2.9 | 59.7** | 7.0 |

*p < 0.05,
**p < 0.01,
***p < 0.001 vs Normal Control and
#p < 0.05,
##p < 0.01, and
###p < 0.001 vs Semaglutide;
One way ANOVA followed by Bonferroni's posttests

### Example 2: Efficacy Study in db/db Mice at 12 nM/Kg Dose

The effect of the compounds as described herein on blood glucose, food intake and body weight were studied in mice. This study was performed in Type 2 diabetes mouse (db/db) model. The animals were divided into 5 treatment groups (n=4/group): a diabetic control group, Cotadutide (12 nM/kg), Compound 3 (12 nM/kg), Compound 4 (12 nM/kg), and Semaglutide (12 nM/kg). Baseline blood glucose was measured from all the animals. All the animals were administered with test item subcutaneously. Blood glucose was measured at 4 hr, 8 hr, 24 hr, 48 hr, 72 hr, and 96 hr post treatment. Delta blood glucose (mM) was calculated.

TABLE 7
Effect on Blood Glucose

| | | Delta Blood Glucose (mM) | | | | | |
|---|---|---|---|---|---|---|---|
| Treatment (n = 4) | | 4 hr | 8 hr | 24 hr | 48 hr | 72 hr | 96 hr |
| Diabetic Control | Mean | −0.24 | 4.28 | 2.47 | 2.90 | 1.78 | 2.68 |
| | SD | 3.24 | 3.54 | 3.79 | 5.03 | 4.79 | 4.97 |
| Cotadutide, 12 | Mean | −9.83** | −9.97*** | −2.82 | 2.89 | 2.06 | 2.43 |
| nM/kg/s.c/single | SD | 4.40 | 4.51 | 1.59 | 4.37 | 4.90 | 2.66 |
| dose | | | | | | | |
| Compound 3, 12 | Mean | −7.79* | −11.18*** | −3.72 | 2.08 | 3.47 | 3.79 |
| nM/kg/s.c/single | SD | 3.98 | 4.38 | 4.18 | 7.59 | 6.49 | 4.37 |
| dose | | | | | | | |
| Compound 4, 12 | Mean | −1.25 | −6.67*** | −2.82 | −0.85 | 0.97 | 1.17 |
| nM/kg/s.c/single | SD | 1.20 | 6.19 | 1.01 | 0.82 | 1.66 | 2.98 |
| dose | | | | | | | |
| Semaglutide, 12 | Mean | −10.07** | −10.51*** | −8.19*** | 1.78 | 2.38 | 2.69 |
| nM/kg/s.c/single | SD | 3.11 | 2.70 | 1.59 | 3.46 | 1.88 | 3.00 |
| dose | | | | | | | |

*p < 0.05,
**p < 0.01,
***p < 0.001 vs. Diabetic Control, two-way ANOVA followed by Bonferroni's post test

### Example 3: Efficacy Study in db/db Mice at 10 nM/kg Dose

The effect of the compounds as described herein on blood glucose, food intake and body weight were studied in mice. This study was performed in Type 2 diabetes mouse (db/db) model. The animals were divided into 2 treatment groups (n=5/group): a diabetic control group and Compound 5 (10 nM/kg). Baseline blood glucose was measured from all the animals. All the animals were administered with test item subcutaneously. Blood glucose was measured at 4 hr, 8 hr, 12 hr, 24 hr, 48 hr, 72 hr, and 96 hr post treatment. Delta blood glucose (mM) was calculated. Body weight changes and cumulative food consumption was measured at 48 hr and 96 hr post treatment.

TABLE 8
Effect on Blood Glucose

| Treatment | Delta Blood Glucose (mM) | | | | | | | |
|---|---|---|---|---|---|---|---|---|
| (n = 5) | | 4 hr | 8 hr | 12 hr | 24 hr | 48 hr | 72 hr | 96 hr |
| Diabetic | Mean | −0.1 | 0.1 | 2.1 | 0.7 | 1.2 | 1.0 | 1.1 |
| Control | SD | 1.2 | 2.8 | 1.7 | 4.1 | 1.8 | 2.2 | 2.3 |
| Compound 5, | Mean | −12.6*** | −9.2*** | −9.7*** | −4.1* | 0.5 | 0.1 | −1.5 |
| 10 | SD | 4.1 | 2.6 | 2.5 | 3.4 | 2.8 | 1.6 | 2.2 |
| nM/kg/s.c/single | | | | | | | | |
| dose | | | | | | | | |

*p < 0.05,
**p < 0.01,
***p < 0.001 vs. Diabetic Control, two-way ANOVA followed by Bonferroni's post test

TABLE 9
Effect on Body Weight

| | Body Weight | | Body Weight | |
|---|---|---|---|---|
| | Change (%) 48 hr | | Change (%) 96 hr | |
| Groups (n = 5) | Mean | SD | Mean | SD |
| Diabetic Control | 1.1 | 0.7 | 1.8 | 1.0 |
| Compound 5, 10 | −2.4*** | 1.2 | −3.1*** | 0.9 |
| nM/kg/s.c/single dose | | | | |

*p < 0.05,
**p < 0.01,
***p < 0.001 vs. Diabetic Control;
One-way ANOVA followed by Dunnett's post test

TABLE 10
Effect on Food Consumption

| | Cumulative Food | | Cumulative Food | |
|---|---|---|---|---|
| | Consumption | | Consumption | |
| | (g) 0-48 hr | | (g) 0-96 hr | |
| Groups (n = 5) | Mean | SD | Mean | SD |
| Diabetic Control | 14.9 | 0.5 | 29.8 | 1.2 |
| Compound 5, 10 nM/kg/s.c/single | 7.5*** | 0.7 | 17.1*** | 1.2 |
| dose | | | | |

*p < 0.05,
**p < 0.01,
***p < 0.001 vs. Diabetic Control;
One-way ANOVA followed by Dunnett's post test

### Example 4: Efficacy Study in db/db Mice at 10 nM/Kg Dose

The effect of the compounds as described herein on blood glucose, food intake and body weight were studied in mice. This study was performed in Type 2 diabetes mouse (db/db) model. The animals were divided into 12 treatment groups (n=4/group): a diabetic control group, Mazdutide (10 nM/kg), Compound 6 (10 nM/kg), Compound 7 (10 nM/kg), Compound 25 (10 nM/kg), and Tirzepatide (10 nM/kg). Baseline blood glucose was measured from all the animals. All the animals were administered with test item subcutaneously. Blood glucose was measured at 4 hr, 8 hr, 12 hr, 24 hr, 48 hr, 72 hr, and 96 hr post treatment. Delta blood glucose (mM) was calculated. Body weight changes and cumulative food consumption was measured at 48 hr and 96 hr post treatment.

TABLE 11
Effect on Blood Glucose

| | Delta Blood Glucose (mM) | | | | | | | |
|---|---|---|---|---|---|---|---|---|
| Treatment (n = 4) | | 4 hr | 8 hr | 12 hr | 24 hr | 48 hr | 72 hr | 96 hr |
| Diabetic Control | Mean | −2.9 | −3.2 | −1.9 | −0.7 | 0.9 | −0.6 | −1.5 |
| | SD | 4.1 | 3.8 | 4.0 | 7.0 | 2.6 | 4.6 | 3.6 |
| Mazdutide, 10 | Mean | −7.3 | −3.2 | −3.9 | −4.1 | −2.8 | 7.3* | 7.6** |
| nM/kg/s.c/single | SD | 2.4 | 2.3 | 3.6 | 3.7 | 5.7 | 2.3 | 2.2 |
| dose | | | | | | | | |
| Compound 6, 10 | Mean | −11.3* | −12.0** | −11.5*** | −9.5** | −8.9* | 2.9 | 5.3 |
| nM/kg/s.c/single | SD | 3.5= | 5.8 | 6.3 | 2.1 | 2.4 | 5.1 | 4.8 |
| dose | | | | | | | | |
| Compound 7, 10 | Mean | −8.4 | −11.0*** | −10.7*** | −8.5** | −2.1 | −0.7 | 1.1 |
| nM/kg/s.c/single | SD | 1.7 | 3.3 | 3.4 | 3.3 | 2.8 | 2.3 | 2.7 |
| dose | | | | | | | | |
| Compound 25, 10 | Mean | −3.6 | −5.2* | −7.6** | 1.5 | 8.6* | 9.4** | 9.7*** |
| nM/kg/s.c/single | SD | 1.8 | 2.0 | 4.3 | 6.3 | 1.8 | 1.7 | 1.7 |
| dose | | | | | | | | |
| Tirzepatide, 10 | Mean | −8.2 | −12.1*** | −14.3*** | −13.9*** | −9.5* | −4.9 | 3.7 |
| nM/kg/s.c/single | SD | 2.1 | 2.5 | 3.1 | 3.6 | 6.5 | 4.1 | 2.9 |
| dose | | | | | | | | |

*p < 0.05,
**p < 0.01,
***p < 0.001 vs. Diabetic Control, two-way ANOVA followed by Bonferroni's post test

TABLE 12
Effect on Body Weight

| | Body Weight | | Body Weight | |
|---|---|---|---|---|
| | Change (%) | | Change (%) | |
| | 48 hr | | 96 hr | |
| Groups (n = 4) | Mean | SD | Mean | SD |
| Diabetic Control | 0.5 | 0.1 | 1.7 | 0.4 |
| Mazdutide, 10 nM/kg/s.c/single | −6.9*** | 0.6 | −5.9*** | 0.8 |
| dose | | | | |
| Compound 6, 10 nM/kg/s.c/single | −6.6*** | 1.0 | −4.1*** | 1.4 |
| dose | | | | |
| Compound 7, 10 nM/kg/s.c/single | −6.1*** | 0.8 | −12.6*** | 1.0 |
| dose | | | | |
| Compound 25, 10 | −5.1*** | 0.5 | −2.3*** | 1.3 |
| nM/kg/s.c/single dose | | | | |
| Tirzepatide, 10 nM/kg/s.c/single | −8.3*** | 1.8 | −2.8*** | 1.4 |
| dose | | | | |

*p < 0.05,
**p < 0.01,
***p < 0.001 vs. Diabetic Control;
One-way ANOVA followed by Dunnett's post test

TABLE 13
Effect on Food Consumption

| | Cumulative Food | | Cumulative Food | |
|---|---|---|---|---|
| | Consumption | | Consumption | |
| | (g) 0-48 hr | | (g) 0-96 hr | |
| Groups (n = 4) | Mean | SD | Mean | SD |
| Diabetic Control | 13.0 | 0.9 | 29.6 | 1.3 |
| Mazdutide, 10 nM/kg/s.c/single | 3.1*** | 1.1 | 11.4*** | 0.6 |
| dose | | | | |
| Compound 6, 10 nM/kg/s.c/single | 4.2*** | 0.3 | 15.2*** | 0.7 |
| dose | | | | |
| Compound 7, 10 nM/kg/s.c/single | 9.4*** | 0.1 | 16.4*** | 0.3 |
| dose | | | | |
| Compound 25, 10 | 8.4*** | 0.2 | 25.4* | 0.6 |
| nM/kg/s.c/single dose | | | | |
| Tirzepatide, 10 nM/kg/s.c/single | 3.8*** | 0.2 | 15.1*** | 0.6 |
| dose | | | | |

*p < 0.05,
**p < 0.01,
***p < 0.001 vs. Diabetic Control;
One-way ANOVA followed by Dunnett's post test

### Example 5: In-Vitro Assays

Stably expressing GLP-1R, GIPR or GCGR cell lines were used to determine in-vitro potency of the compounds as described herein. Signaling by the GLP-1R, GIP-R, and GCG-R involves activation of adenylate cyclase and cAMP production. Hit Hunter® cAMP assays monitor the activation of GLP-1R, GIPR or GCGR via Gi and Gs secondary messenger signaling using a technology developed by DiscoverX which is called Enzyme Fragment Complementation (EFC) with β-galactosidase (β-Gal) as the functional reporter. The enzyme is split into two complementary portions: EA for Enzyme Acceptor and ED for Enzyme Donor. ED is fused to cAMP and in the assay competes with cAMP generated by cells for binding to a cAMP-specific antibody. Active P-Gal is formed by complementation of exogenous EA to any unbound ED cAMP. Active enzyme can then convert a chemiluminescent substrate, generating an output signal detectable on a standard microplate reader.

Three different assays were performed using cells expressing either of the three receptors. cAMP Hunter cell lines were expanded from freezer stocks according to standard procedures. Cells were seeded in a total volume of 20 μL into white walled, 384-well microplates and incubated at 37 °C for the appropriate time prior to testing. Media was aspirated and cells were then treated with 15 μL of cAMP conjugated antibody and 5 μL of test compound. After appropriate compound incubation, assay signal was generated through incubation with 20 μL cAMP-ED cell lysis cocktail for one hour followed by incubation with 20 μL cAMP-EA reagent for three hours at room temperature. Free cAMP-ED available in the system compliment with the free cAMP-EA to form Active R-Gal that reacts with the substrate to give chemiluminescent signal. Microplates were read following signal generation with a PerkinElmer Envision™ instrument for chemiluminescent signal detection. The amount of signal is directly proportional to the concentration of cAMP generated due to response. Different concentrations of the sample was used (different for different compound) to generate log Concentration to % Effect curve. Four parametric logistic curve was generated and EC50 was determined. Appropriate assay reference was used (Exendin-4 for GLP-1R, GIP for GIPR and Glucagon for GCGR) for each assay.

Cellular cAMP Assay of Mazdutide, Compound 6, Compound 7, and Compound 8 was performed, and the half effective concentrations on GLP-1R-expressing cells and GIPR-expressing cells was as mentioned in Table 14.

TABLE 14

| | | EC50 value | | |
|---|---|---|---|---|
| Compound | Concentration | GCG | GIP | GLP-1 |
| Mazdutide | (pM) | 21.11 | NA | 11.15 |
| Compound 6 | (pM) | 17.28 | NA | 11.76 |
| Compound 7 | (pM) | 15.24 | NA | 8.72 |
| Compound 8 | (pM) | 90.63 | NA | 11.55 |

What is claimed is:

1. A polypeptide or a pharmaceutically acceptable salt thereof comprising the amino acid sequence:
(SEQ ID NO: 1)
X1-X2-X3-G-T-F-T-S-D-X10-S-X12-X13-L-D-X16-X17-
X18-X19-X20-X21-F-X23-X24-X25-L-X27-X28-X29-X30-
X31-X32-X33-X34-X35-X36-X37-X38-X39
wherein:
X1 is H;
X2 is D-Ser(OMe), Aib or D-S;
X3 is Q;
X10 is K or Y;
X12 is E, K or I;
X13 is Y, S(OMe), nor-V, nor-L, or αMe-L;
X16 is S, E or A;
X17 is E, R or K;
X18 is R, K or A;
X19 is A;
X20 is R, Q or K;
X21 is D or E;
X23 is V or I;
X24 is A, Q or E;
X25 is W;
X27 is E or L;
X28 is A, D or E;
X29 is G or T;
X30 is absent or G;
X31 is absent or P;
X32 is absent or S;
X33 is absent or S;
X34 is absent or G;
X35 is absent;
X36 is absent;
X37 is absent;
X38 is absent; and
X39 is absent;
wherein the acid group of the C-terminal amino acid is a free carboxylic acid group or is amidated as C-terminal primary amide; and
with a proviso that at least one of X10 and X20 is K, and further provided at least one of said K comprises a side chain amino (ε amino) group acylated with a moiety of the formula selected from:

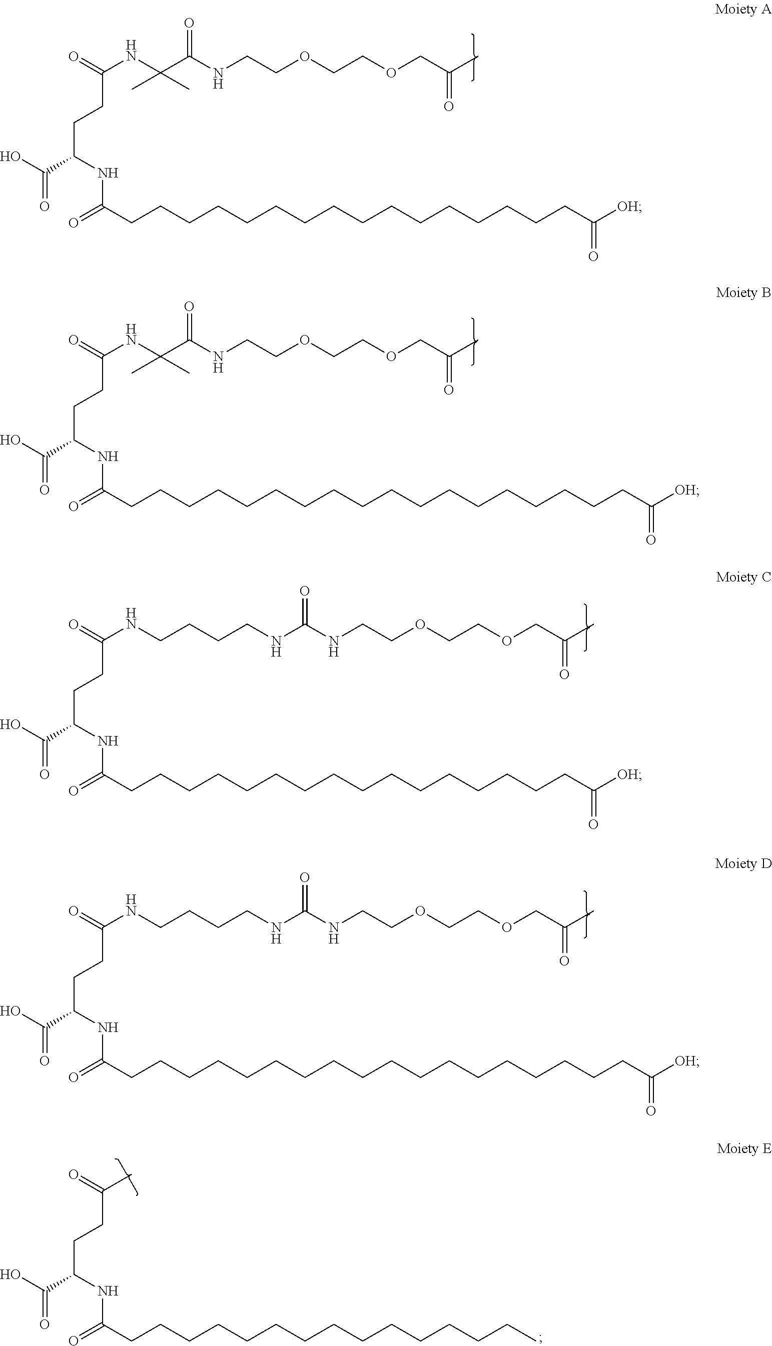 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O ; CCCCCCCCCCCCCCCC(=O)N[C@@H](CCC(C)=O)C(=O)O]

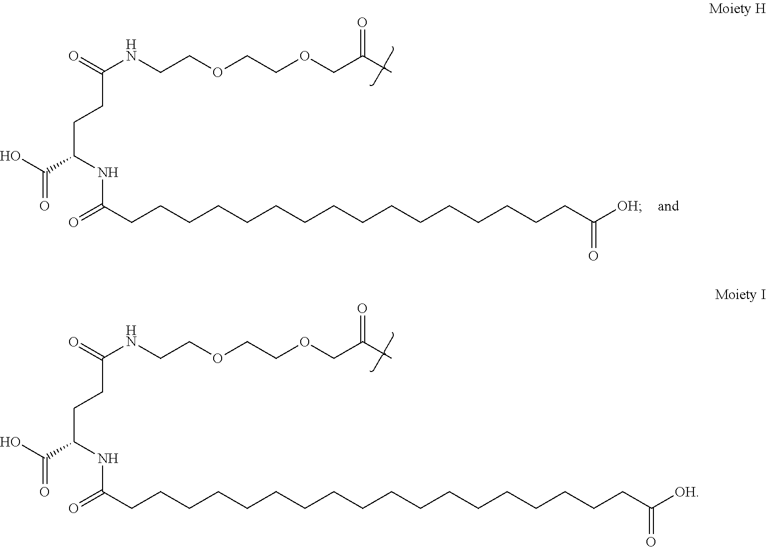 [SMILES: CC(=O)COCCOCCNC(=O)CC[C@H](NC(=O)CCCCCCCCCCCCCCCCC(=O)O)C(=O)O ; CC(=O)COCCOCCNC(=O)CC[C@H](NC(=O)CCCCCCCCCCCCCCCCCCC(=O)O)C(=O)O]

2. A polypeptide or a pharmaceutically acceptable salt thereof comprising the amino acid sequence:
(SEQ ID NO: 2)
X1-X2-X3-G-T-F-T-S-D-X10-S-X12-X13-L-D-X16-X17-
X18-X19-X20-X21-F-X23-X24-X25-L-X27-X28-X29-X30-
X31-X32-X33-X34-X35-X36-X37-X38-X39
wherein:
X1 is H;
X2 is S, D-Ser(OMe), Aib, or D-S;
X3 is Q;
X10 is K or Y;
X12 is E, K or I;
X13 is Y, S(OMe), nor-V, nor-L, or αMe-L;
X16 is S, E or A;
X17 is E, R or K;
X18 is R, K or A;
X19 is A;
X20 is R, Q or K,
X21 is D or E;
X23 is V;
X24 is A, Q or E;
X25 is W;
X27 is E or L;
X28 is A, D or E;
X29 is G or T;
X30 is absent or G;
X31 is absent or P;
X32 is absent or S;
X33 is absent or S;
X34 is absent or G;
X35 is absent;
X36 is absent;
X37 is absent;
X38 is absent; and
X39 is absent;
wherein the acid group of the C-terminal amino acid is a free carboxylic acid group or is amidated as C-terminal primary amide; and
with a proviso that at least one of X10 and X20 is K, and further provided that at least one of said K comprises a side chain amino (ε amino) group acylated with a moiety of the formula selected from:

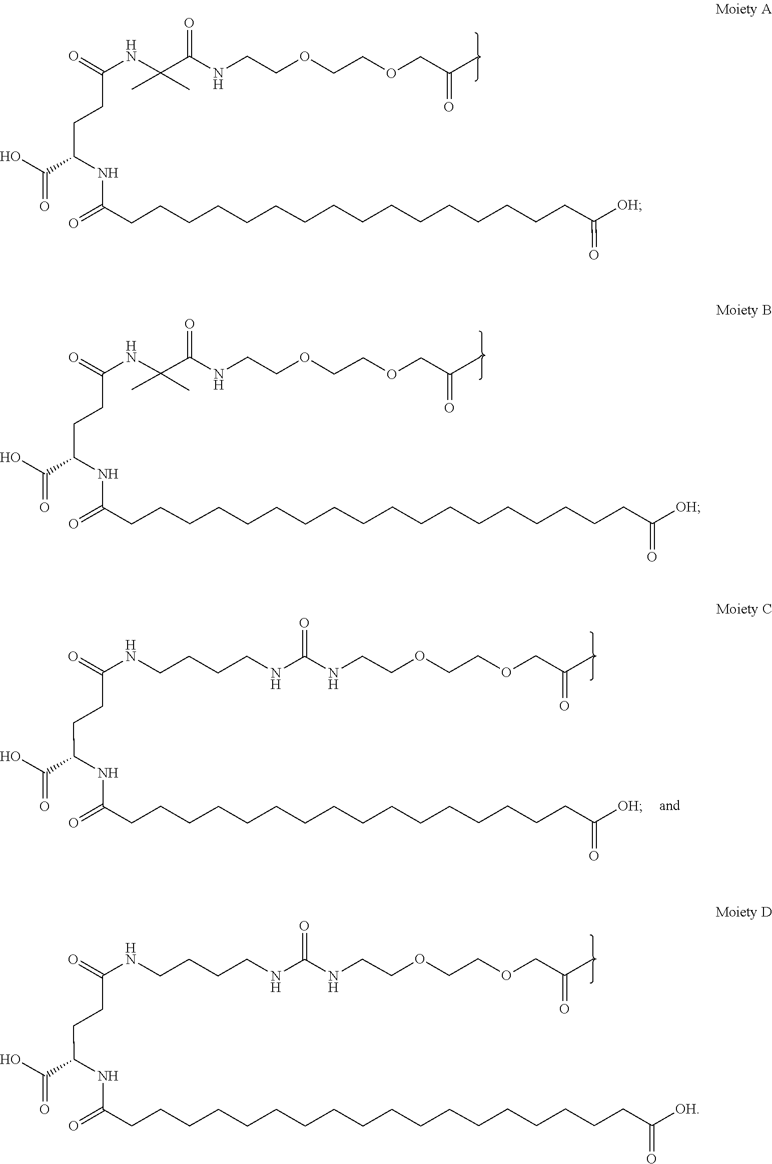 [SMILES: CC(=O)COCCOCCNC(=O)C(C)(C)NC(=O)CC[C@H](NC(=O)CCCCCCCCCCCCCCCCC(=O)O)C(=O)O ; CCC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

3. The polypeptide according to claim 1, wherein:
X2 is D-Ser(OMe) or Aib;
X10 is K;
X12 is E;
X13 is Y;
X16 is S;
X17 is E;
X18 is R;
X20 is R;
X21 is D;
X24 is A;
X27 is E;
X28 is A;
X29 is G;
X30 is G; and
X31, X32, X33 and X34 are absent;
wherein the side chain amino (ε amino) group of K at position X10 is acylated with a moiety of the formula selected from:

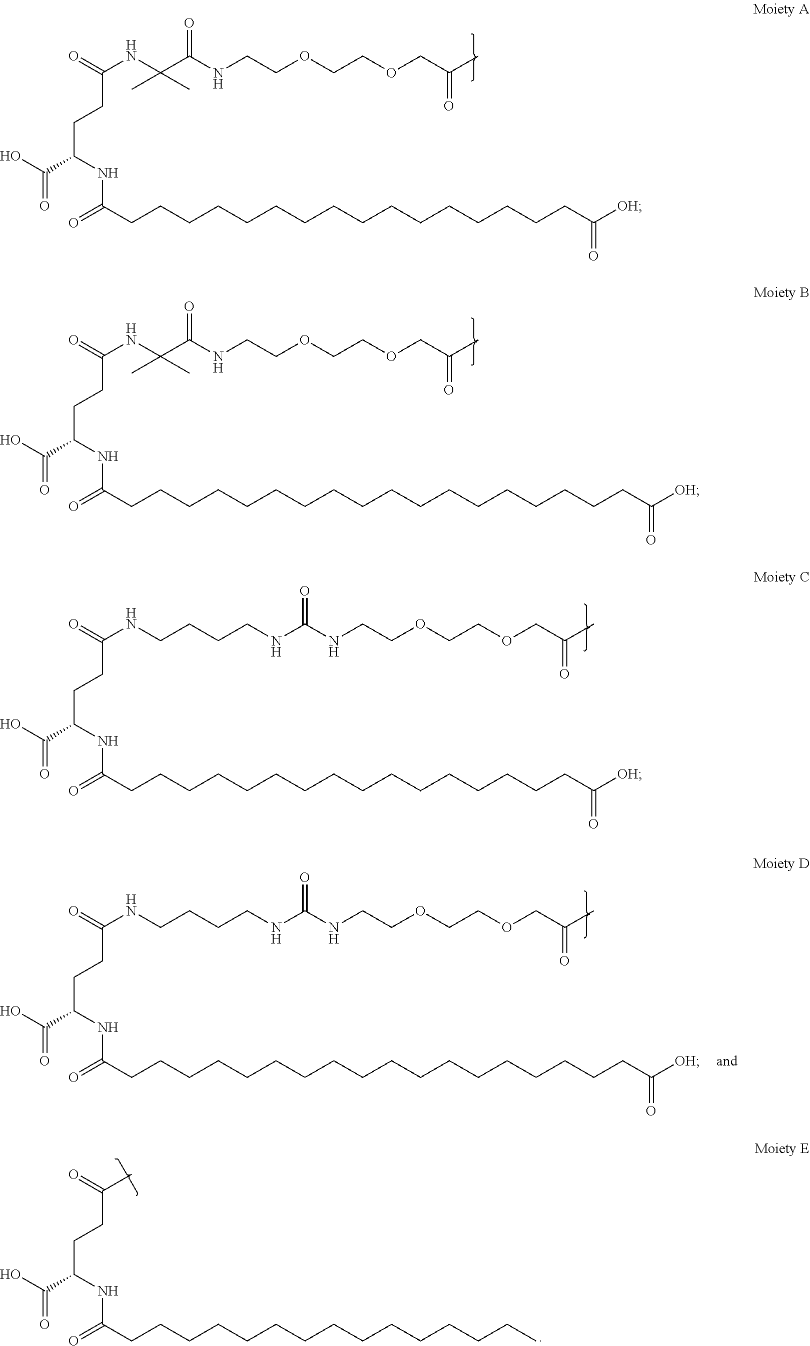 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O ; CCCCCCCCCCCCCCCC(=O)N[C@@H](CCC(C)=O)C(=O)O]

4. The polypeptide according to claim 1 or 2, wherein:
X2 is D-S,
X10 is K,
X12 is K,
X13 is Y;
X16 is A,
X17 is R,
X18 is A,
X20 is Q;
X21 is D,
X24 is Q;
X27 is L,
X28 is D,
X29 is T, and
X31, X32, X33 and X34 are absent;
wherein the side chain amino (ε amino) group of K at position X10 is acylated with a moiety of the formula selected from:

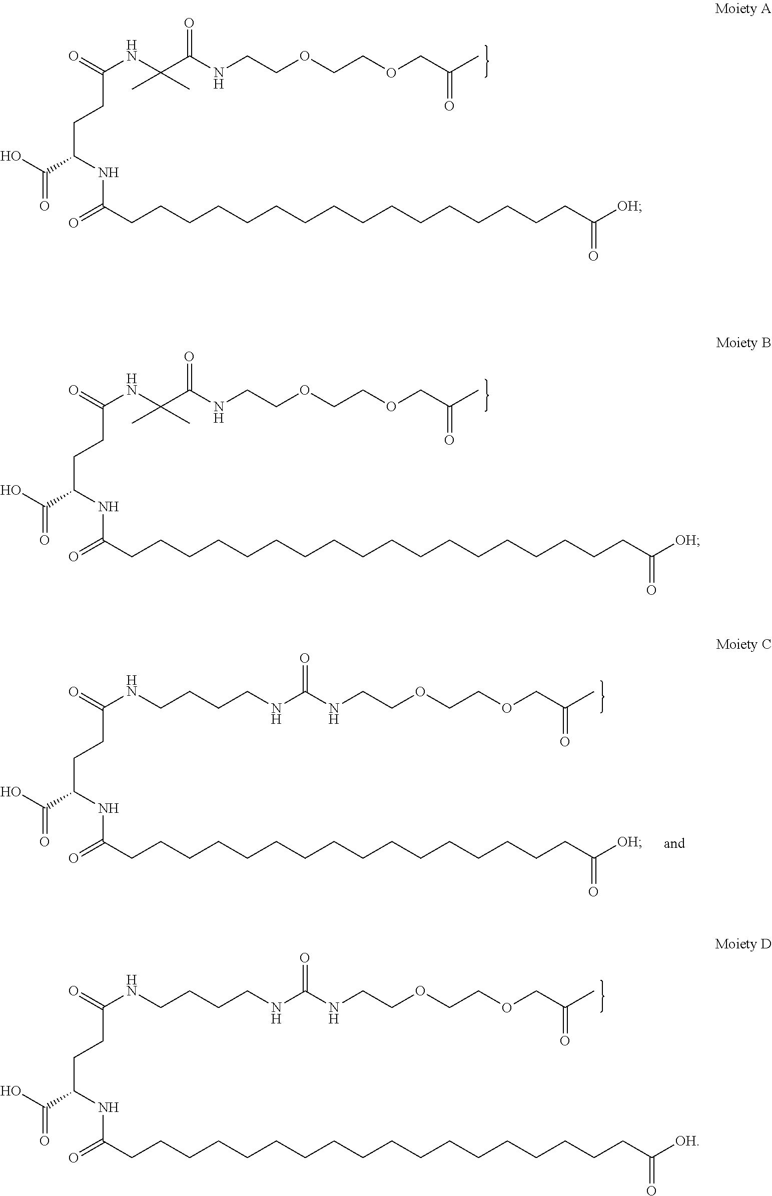 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

5. The polypeptide according to claim 1 or 2, wherein:
X2is Aib;
X10 is Y;
X12 is K or I;
X13 is Y or nor-V, nor-L, or αMe-L;
X16 is E,
X17 is K,
X18 is K,
X20 is K,
X21 is E,
X24 is E,
X27 is L,
X28 is E,
X29 is G,
X30 is G
X31 is P,
X32 is S,
X33 is S, and
X34 is G;
wherein the side chain amino (ε amino) group of K at position X20 is acylated with a moiety of the formula selected from:

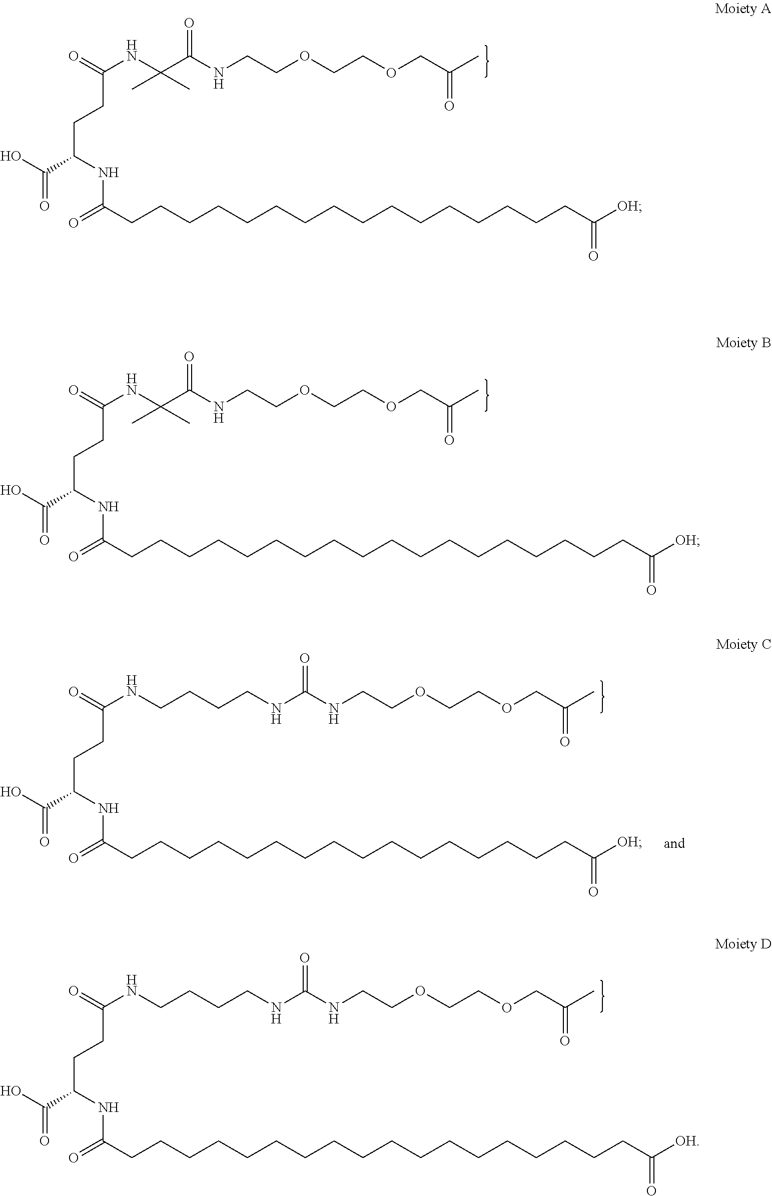 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

6. A polypeptide or a pharmaceutically acceptable salt thereof comprising the amino acid sequence:
(SEQ ID NO: 3)
H-X2-Q-G-T-F-T-S-D-X10-S-E-Y-L-D-S-E-R-A-R-D-F-V-
A-W-L-E-A-G-G
wherein:
X2 is S, D-S(OMe) or Aib; and
X10 is K;
wherein the acid group of the C-terminal amino acid is a free carboxylic acid group or is amidated as C-terminal primary amide; and
wherein the side chain amino (ε amino) group of K at position X10 is acylated with a moiety of the formula selected from: 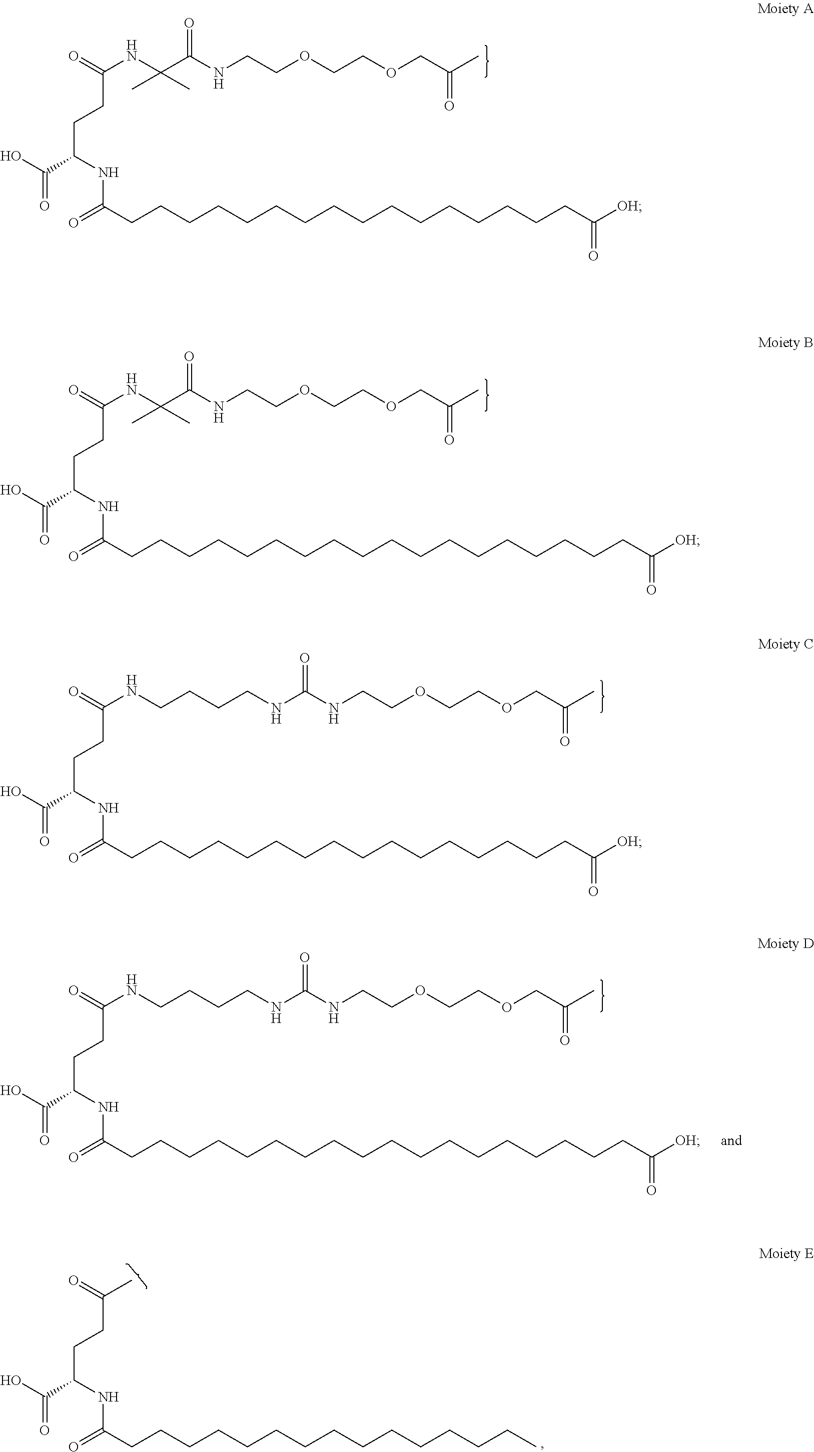 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O ; CCCCCCCCCCCCCCCC(=O)N[C@@H](CCC(C)=O)C(=O)O]
with a proviso that the polypeptide is not SEQ ID NO: 6.

7. The polypeptide according to claim 6, wherein:
X2 is S;
wherein the side chain amino (ε amino) group of K at position X10 is acylated with a moiety of the formula selected from:

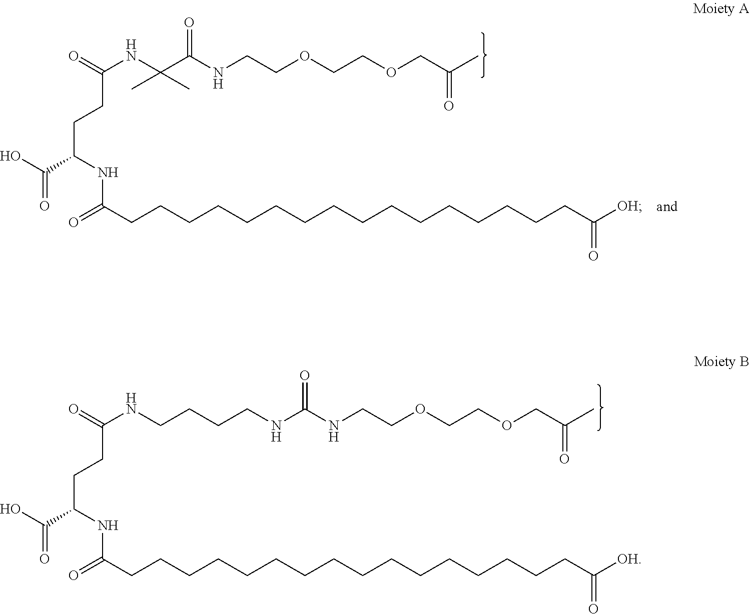 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O]

8. The polypeptide according to claim 6, wherein:
X2 is D-Ser(OMe);
wherein the side chain amino (ε amino) group of K at position X10 is acylated with a moiety of the formula:

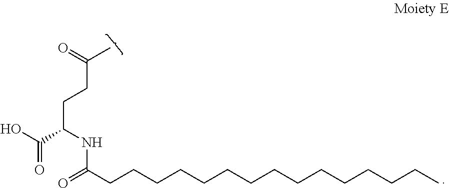 [SMILES: CCCCCCCCCCCCCCCC(=O)N[C@@H](CCC(C)=O)C(=O)O]

9. The polypeptide according to claim 6, wherein:
X2 is Aib;
wherein the side chain amino (ε amino) group of K at position X10 is acylated with a moiety of the formula selected from:

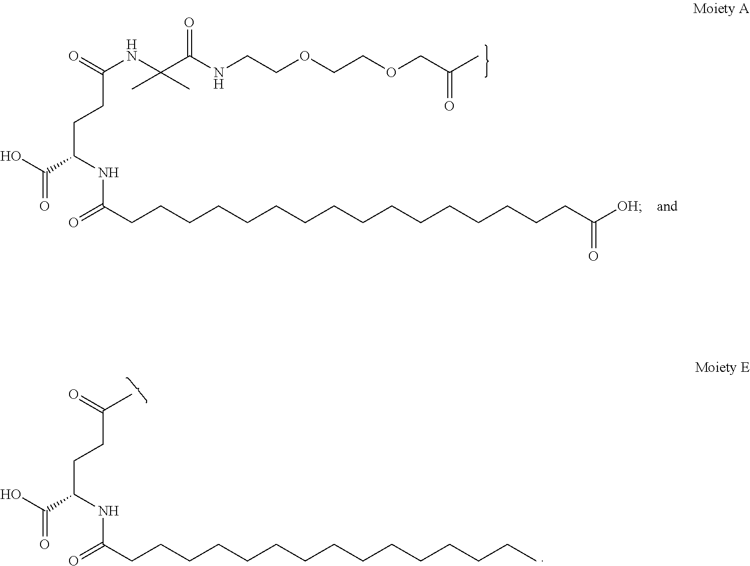 [SMILES: CC(=O)COCCOCCNC(=O)C(C)(C)NC(=O)CC[C@H](NC(=O)CCCCCCCCCCCCCCCCC(=O)O)C(=O)O ; CCCCCCCCCCCCCCCC(=O)N[C@@H](CCC(C)=O)C(=O)O]

10. A polypeptide or a pharmaceutically acceptable salt thereof comprising the amino acid sequence:
(SEQ ID NO: 4)
H-Aib-Q-G-T-F-T-S-D-Y-S-X12-X13-L-D-E-K-K-A-X20-
E-F-V-E-W-L-L-E-G-G-P-S-S-G
wherein: PP4,N
X12 is K or I;
X13 is Y or nor-V; and
X20 is K;
wherein the acid group of the C-terminal amino acid is a free carboxylic acid group or is amidated as C-terminal primary amide;
wherein the side chain amino (ε amino) group of K at position X20 is acylated with a moiety of the formula selected from:

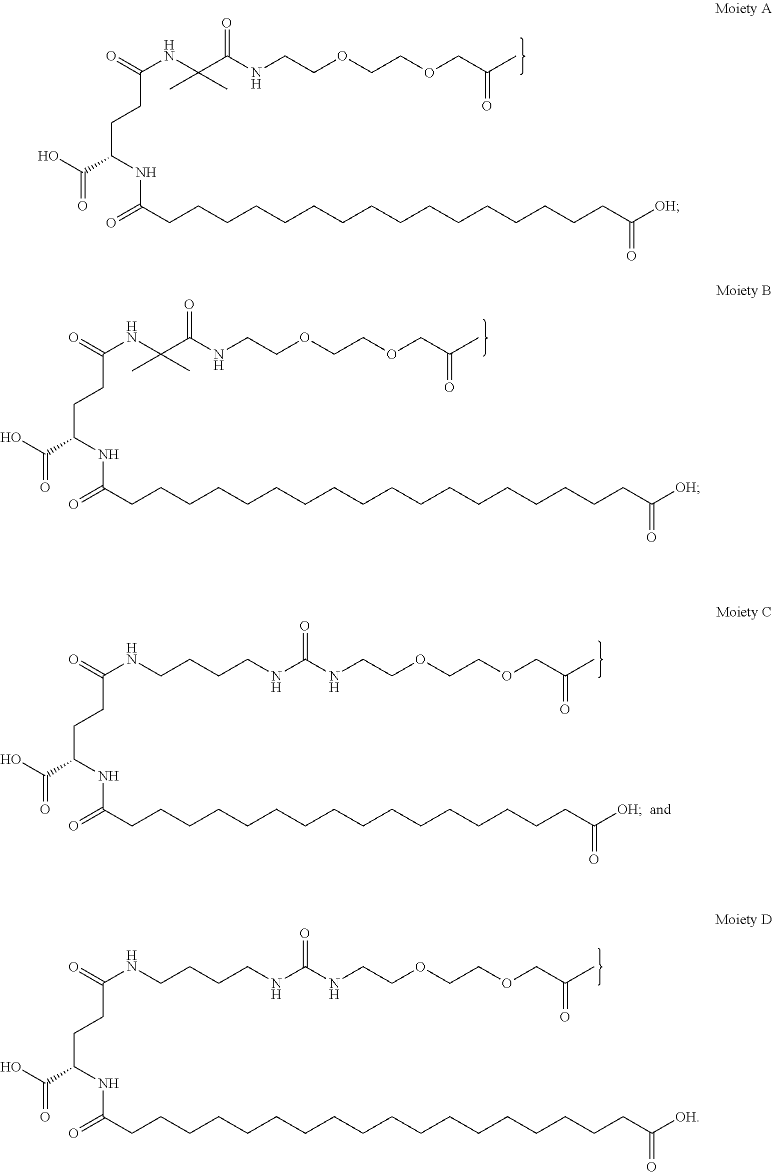

11. The polypeptide according to claim 10, wherein:
X12 is K; and
X13 is Y;
wherein the side chain amino (ε amino) group of K at position X20 is acylated with a moiety of the formula selected from:

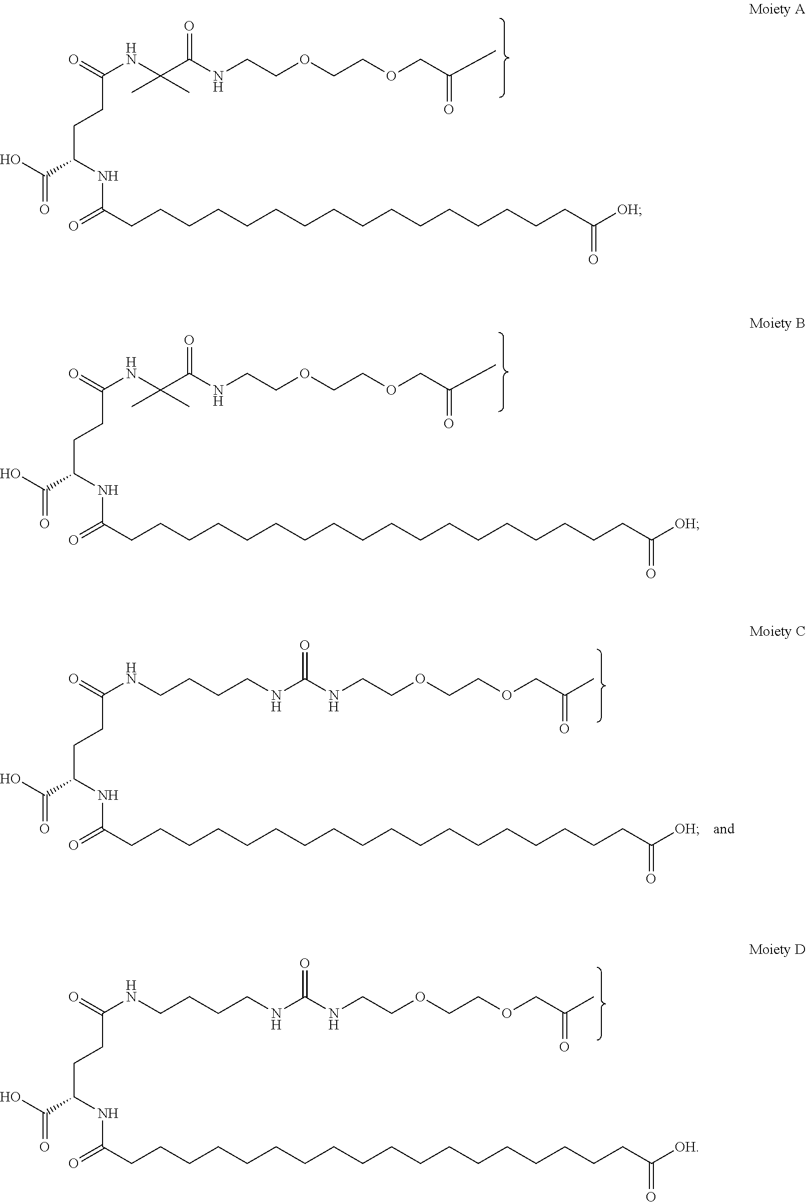 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

12. The polypeptide according to claim 10, wherein:
X12 is I; and
X13 is nor-V;
wherein the side chain amino (ε amino) group of K at position X20 is acylated with a moiety of the formula:

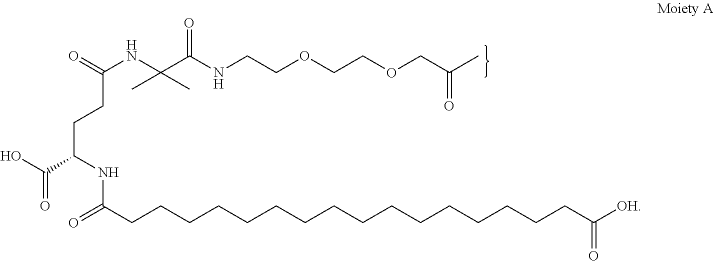 [SMILES: CC(=O)COCCOCCNC(=O)C(C)(C)NC(=O)CC[C@H](NC(=O)CCCCCCCCCCCCCCCCC(=O)O)C(=O)O]

13. The polypeptide according to claim 10, wherein:
X12 is K; and
X13 is nor-V;
wherein the side chain amino (ε amino) group of K at position X20 is acylated with a moiety of the formula:

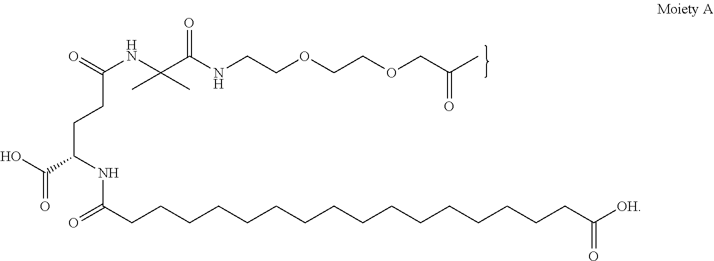 [SMILES: CC(=O)COCCOCCNC(=O)C(C)(C)NC(=O)CC[C@H](NC(=O)CCCCCCCCCCCCCCCCC(=O)O)C(=O)O]

14. A polypeptide or a pharmaceutically acceptable salt thereof comprising the amino acid sequence:
(SEQ ID NO: 5)
H-(DSer)-Q-G-T-F-T-S-D-X10-S-K-Y-L-D-A-R-A-A-Q-D-
F-V-Q-W-L-L-D-T
wherein X10 is K;
wherein the acid group of the C-terminal amino acid is a free carboxylic acid group or is amidated as C-terminal primary amide; and
wherein the side chain amino (ε amino) group of K at position X10 is acylated with a moiety of the formula selected from:

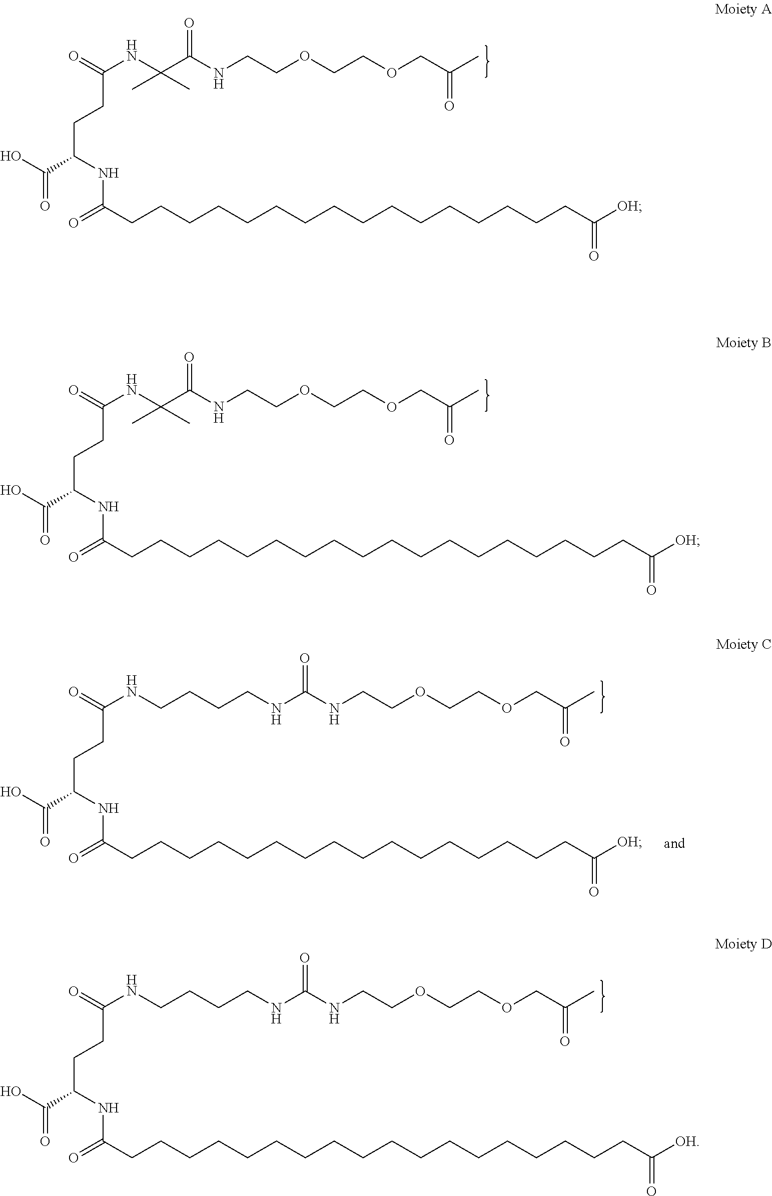 [SMILES: CC(=O)COCCOCCNC(=O)C(C)(C)NC(=O)CC[C@H](NC(=O)CCCCCCCCCCCCCCCCC(=O)O)C(=O)O ; CC(=O)COCCOCCNC(=O)C(C)(C)NC(=O)CC[C@H](NC(=O)CCCCCCCCCCCCCCCCCCC(=O)O)C(=O)O ; CC(=O)COCCOCCNC(=O)NCCCCNC(=O)CC[C@H](NC(=O)CCCCCCCCCCCCCCCCC(=O)O)C(=O)O ; CC(=O)COCCOCCNC(=O)NCCCCNC(=O)CC[C@H](NC(=O)CCCCCCCCCCCCCCCCCCC(=O)O)C(=O)O]

15. A polypeptide which is selected from:

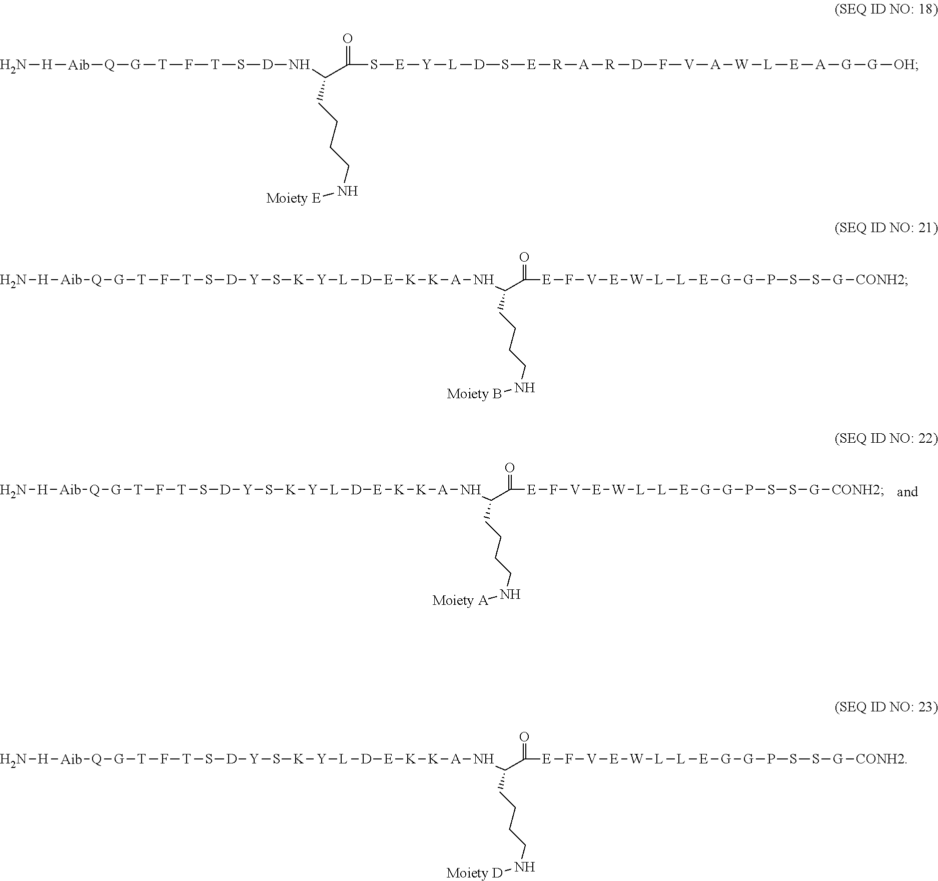

16. An incretin analog comprising:
a peptide residue having the sequence X¹-Gln-Gly-Thr-Phe-Thr-Ser-Asp-Lys (SEQ ID NO:29), wherein X¹ represents Aib or Ser(OMe), and wherein the lysine comprises a fatty acid protracting group attached to the lysine ε-nitrogen; and
a Gly-Gly-OH peptide residue indirectly attached to the carboxy of the lysine.

17. An incretin analog comprising:
a peptide residue having the sequence X¹-Gln-Gly-Thr-Phe-Thr-Ser-Asp-Lys (SEQ ID NO:29), wherein X¹ represents Aib or Ser(OMe), and wherein the lysine comprises a group of formula (I) attached to the lysine ε-nitrogen, 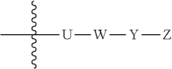
wherein:
U is absent or represents —C(O)—CH₂—O—(CH₂)₂—O—(CH₂)₂—NH—}, wherein } is point of attachment to W;
W represents:
—C(O)—CH₂—O—(CH₂)₂—O—(CH₂)₂—NH—],
—C(O)—NH—(CH₂)₃₋₄—NH—], —C(O)—C(CH₃)₂—NH—], or 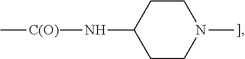
wherein ] is point of attachment to Y;
Y is absent or represents —C(O)—(CH₂)₂—CH(CO₂H)NH—or —C(O)CH((CH₂)ₓCO₂H)NH—, wherein x is 1, 2 or 3, and wherein — is point of attachment to Z; and
Z represents —C(O)—(CH₂)ₙ—COOH or —C(O)—(CH₂)ₙ—CH₃, wherein n is an integer from 14-20; and
a Gly-Gly-OH peptide residue indirectly attached to the carboxy of the lysine.

18. An incretin analog according to claim 16 or claim 17, wherein the lysine is attached to the Gly-Gly-OH residue by a peptide residue comprising 18 amino acids.

19. An incretin analog comprising:
a peptide residue having the sequence Aib-Gln-Gly-Thr-Phe-Thr-Ser-Asp (SEQ ID NO:30);
a lysine residue indirectly attached to the carboxy of the Aib-Gln-Gly-Thr-Phe-Thr-Ser-Asp (SEQ ID NO:30) residue wherein the lysine comprises a group of formula (I) attached to the lysine ε-nitrogen; 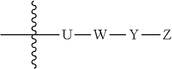
wherein:
U is absent or represents —C(O)—CH₂—O—(CH₂)₂—O—(CH₂)₂—NH—} wherein } is point of attachment to W;
W represents
—C(O)—CH₂—O—(CH₂)₂—O—(CH₂)₂—NH—], or
—C(O)—NH—(CH₂)₃₋₄—NH—], —C(O)—C(CH₃)₂—NH—],
wherein ] is point of attachment to Y;
Y is absent or represents —C(O)—(CH₂)₂—CH(CO₂H)NH—or —C(O)CH((CH₂)ₓCO₂H)NH—, wherein x is 1, 2 or 3, and wherein — is point of attachment to Z; and
Z represents —C(O)—(CH₂)ₙ—COOH or —C(O)—(CH₂)ₙ—CH₃, wherein n is an integer from 14-20; and
a Gly-Gly-Pro-Ser-Ser-Gly-CONH₂ peptide residue indirectly attached to the carboxy of the lysine.

20. An incretin analog according to any one of claim 19, wherein the lysine is attached to the Aib-Gln-Gly-Thr-Phe-Thr-Ser-Asp (SEQ ID NO:30) residue by a peptide residue comprising 10 amino acids.

21. A pharmaceutical composition comprising a pharmaceutically acceptable carrier and an incretin analog of any one of claims 1-20.

22. A method of treating obesity comprising administering to a patient in need of such treatment an incretin analog of any one of claims 1-20.

23. A method of treating Type 2 diabetes mellitus (T2DM) comprising administering to a patient in need of such treatment an incretin analog of any one of claims 1-20.

24. A method of treating metabolic syndrome comprising administering to a patient in need of such treatment an incretin analog of any one of claims 1-20.

25. A method of treating metabolic dysfunction-associated steatotic liver disease (MASLD), comprising administering to a patient in need of such treatment an incretin analog of any one of claims 1-20.

26. A method of treating metabolic dysfunction-associated steatohepatitis (MASH), comprising administering to a patient in need of such treatment an incretin analog of any one of claims 1-20.

27. A method of treating neurodegenerative disorders comprising administering to a patient in need of such treatment an incretin analog of any one of claims 1-20.

28. A method of treating fibrosis comprising administering to a patient in need of such treatment an incretin analog of any one of claims 1-20.

29. A method of reducing cardiovascular risks comprising administering to a patient in need of such treatment an incretin analog of any one of claims 1-20.

30. A method of treating hyperlipidemia/dyslipidemia comprising administering to a patient in need of such treatment an incretin analog of any one of claims 1-20.
